SEQUENCE LISTING (91 sequences)

SEQ ID NO: 1 - protein, 11 aa, Artificial Sequence
  Synthetic Construct
RASENIYXNLA

SEQ ID NO: 2 - protein, 7 aa, Artificial Sequence
  Synthetic Construct
AATQLAX

SEQ ID NO: 3 - protein, 9 aa, Artificial Sequence
  Synthetic Construct
QHFWITPWT

SEQ ID NO: 4 - protein, 5 aa, Artificial Sequence
  Synthetic Construct
XYXMS

SEQ ID NO: 5 - protein, 17 aa, Artificial Sequence
  Synthetic Construct
XISNGGSTTXYPGTVEG

SEQ ID NO: 6 - protein, 9 aa, Artificial Sequence
  Synthetic Construct
HRTNWDFDY

SEQ ID NO: 7 - protein, 11 aa, Artificial Sequence
  Synthetic Construct
RASENIYSNLA

SEQ ID NO: 8 - protein, 11 aa, Artificial Sequence
  Synthetic Construct
RASENIYWNLA

SEQ ID NO: 9 - protein, 11 aa, Artificial Sequence
  Synthetic Construct
RASENIYYNLA

SEQ ID NO: 10 - protein, 7 aa, Artificial Sequence
  Synthetic Construct
AATQLAD

SEQ ID NO: 11 - protein, 7 aa, Artificial Sequence
  Synthetic Construct
AATQLAE

SEQ ID NO: 12 - protein, 7 aa, Artificial Sequence
  Synthetic Construct
AATQLAS

SEQ ID NO: 13 - protein, 5 aa, Artificial Sequence
  Synthetic Construct
SYSMS

SEQ ID NO: 14 - protein, 5 aa, Artificial Sequence
  Synthetic Construct
FYSMS

SEQ ID NO: 15 - protein, 5 aa, Artificial Sequence
  Synthetic Construct
SYLMS

SEQ ID NO: 16 - protein, 5 aa, Artificial Sequence
  Synthetic Construct
SYKMS

SEQ ID NO: 17 - protein, 5 aa, Artificial Sequence
  Synthetic Construct
FYLMS

SEQ ID NO: 18 - protein, 17 aa, Artificial Sequence
  Synthetic Construct
YISNGGSTTYYPGTVEG

SEQ ID NO: 19 - protein, 17 aa, Artificial Sequence
  Synthetic Construct
YISNGGSTTDYPGTVEG

SEQ ID NO: 20 - protein, 17 aa, Artificial Sequence
  Synthetic Construct
RISNGGSTTDYPGTVEG

SEQ ID NO: 21 - protein, 17 aa, Artificial Sequence
  Synthetic Construct
VISNGGSTTDYPGTVEG

SEQ ID NO: 22 - protein, 11 aa, Artificial Sequence
  Synthetic Construct
RASQEISGYFS

SEQ ID NO: 23 - protein, 11 aa, Artificial Sequence
  Synthetic Construct
RASQEIGGYLS

SEQ ID NO: 24 - protein, 11 aa, Artificial Sequence
  Synthetic Construct
RASVNIYSNLA

SEQ ID NO: 25 - protein, 7 aa, Artificial Sequence
  Synthetic Construct
AASTLDS

SEQ ID NO: 26 - protein, 7 aa, Artificial Sequence
  Synthetic Construct
AATNLAE

SEQ ID NO: 27 - protein, 9 aa, Artificial Sequence
  Synthetic Construct
LQYANYPYT

SEQ ID NO: 28 - protein, 9 aa, Artificial Sequence
  Synthetic Construct
QHFWVTPYT

SEQ ID NO: 29 - protein, 6 aa, Artificial Sequence
  Synthetic Construct
ITYLIE

SEQ ID NO: 30 - protein, 6 aa, Artificial Sequence
  Synthetic Construct
TNYLID

SEQ ID NO: 31 - protein, 5 aa, Artificial Sequence
  Synthetic Construct
GYGVN

SEQ ID NO: 32 - protein, 17 aa, Artificial Sequence
  Synthetic Construct
VIHPGSGNSHYNEKFKG

SEQ ID NO: 33 - protein, 17 aa, Artificial Sequence
  Synthetic Construct
AIHPGSGRTHYNEKFKG

SEQ ID NO: 34 - protein, 16 aa, Artificial Sequence
  Synthetic Construct
MIWDDGTTDGDSALRS

SEQ ID NO: 35 - protein, 13 aa, Artificial Sequence
  Synthetic Construct
SGSSSFDYYAMDF

SEQ ID NO: 36 - protein, 13 aa, Artificial Sequence
  Synthetic Construct
SGGSSFDYYAMDY

SEQ ID NO: 37 - protein, 9 aa, Artificial Sequence
  Synthetic Construct
GDYGYAMDY

SEQ ID NO: 38 - protein, 107 aa, Artificial Sequence
  Synthetic Construct
DIQMTQSPSSLSASVGDRVTITCRASENIYSNLAWYQQKPGKAPKLVVYAATQLADGVPSRFSGSGSGTDYTLTISSLQPEDFATYYCQHFWITPWTFGQGTKVEIK

SEQ ID NO: 39 - protein, 107 aa, Artificial Sequence
  Synthetic Construct
DIQMTQSPSSLSASVGDRVTITCRASENIYWNLAWYQQKPGKAPKLVVYAATQLADGVPSRFSGSGSGTDYTLTISSLQPEDFATYYCQHFWITPWTFGQGTKVEIK

SEQ ID NO: 40 - protein, 107 aa, Artificial Sequence
  Synthetic Construct
DIQMTQSPSSLSASVGDRVTITCRASENIYYNLAWYQQKPGKAPKLVVYAATQLADGVPSRFSGSGSGTDYTLTISSLQPEDFATYYCQHFWITPWTFGQGTKVEIK

SEQ ID NO: 41 - protein, 107 aa, Artificial Sequence
  Synthetic Construct
DIQMTQSPSSLSASVGDRVTITCRASENIYYNLAWYQQKPGKAPKLLIYAATQLAEGVPSRFSGSGSGTDYTLTISSLQPEDFATYYCQHFWITPWTFGQGTKVEIK

SEQ ID NO: 42 - protein, 107 aa, Artificial Sequence
  Synthetic Construct
DIQMTQSPSSLSASVGDRVTITCRASENIYYNLAWYQQKPGKAPKLLIYAATQLADGVPSRFSGSGSGTDYTLTISSLQPEDFATYYCQHFWITPWTFGQGTKVEIK

SEQ ID NO: 43 - protein, 107 aa, Artificial Sequence
  Synthetic Construct
DIQMTQSPSSLSASVGDRVTITCRASENIYWNLAWYQQKPGKAPKLLIYAATQLADGVPSRFSGSGSGTDYTLTISSLQPEDFATYYCQHFWITPWTFGQGTKVEIK

SEQ ID NO: 44 - protein, 107 aa, Artificial Sequence
  Synthetic Construct
DIQMTQSPSSLSASVGDRVTITCRASENIYYNLAWYQQKPGKAPKLLIYAATQLASGVPSRFSGSGSGTDYTLTISSLQPEDFATYYCQHFWITPWTFGQGTKVEIK

SEQ ID NO: 45 - protein, 107 aa, Artificial Sequence
  Synthetic Construct
DIQMTQSPSSLSASVGDRVTITCRASENIYWNLAWYQQKPGKAPKLLIYAATQLASGVPSRFSGSGSGTDYTLTISSLQPEDFATYYCQHFWITPWTFGQGTKVEIK

SEQ ID NO: 46 - protein, 107 aa, Artificial Sequence
  Synthetic Construct
DIQMTQSPSSLSASVGDRVTITCRASENIYSNLAWYQQKPGKAPKLLIYAATQLADGVPSRFSGSGSGTDYTLTISSLQPEDFATYYCQHFWITPWTFGQGTKVEIK

SEQ ID NO: 47 - protein, 107 aa, Artificial Sequence
  Synthetic Construct
DIQMTQSPSSLSASVGDRVTITCRASENIYSNLAWYQQKPGKAPKLLIYAATQLAEGVPSRFSGSGSGTDYTLTISSLQPEDFATYYCQHFWITPWTFGQGTKVEIK

SEQ ID NO: 48 - protein, 107 aa, Artificial Sequence
  Synthetic Construct
DIQMTQSPSSLSASVGDRVTITCRASENIYSNLAWYQQKPGKAPKLLIYAATQLASGVPSRFSGSGSGTDYTLTISSLQPEDFATYYCQHFWITPWTFGQGTKVEIK

SEQ ID NO: 49 - protein, 107 aa, Mus musculus
DIQMTQSPASLSVSVGETVTITCRASENIYSNLAWYQQKQGKSPQLVVYAATQLADGVPSRFSGSGSGTQYSLKINSLQSEDFGSYYCQHFWITPWTFGGGTKLEIK

SEQ ID NO: 50 - protein, 107 aa, Mus musculus
DIQMTQSPSSLSASLGERVSLTCRASQEISGYFSWLQQKPDGTIKRLIYAASTLDSGVPKRFSGSRSGSDYSLTISSLESEDFADYYCLQYANYPYTFGGGTKLEIK

SEQ ID NO: 51 - protein, 107 aa, Mus musculus
DIQMTQSPSSLSASLGERVSLTCRASQEIGGYLSWLQQKPDGTFKRLIYAASTLDSGVPKRFSGSRSGSDYSLTISSLESEDFADYYCLQYANYPYTFGGGTKLEIK

SEQ ID NO: 52 - protein, 107 aa, Artificial Sequence
  Synthetic Construct
DIQMTQSPSSLSASVGDRVTITCRASQEISGYFSWLQQKPGKAPKRLIYAASTLDSGVPSRFSGSRSGSDYTLTISSLQPEDFATYYCLQYANYPYTFGQGTKVEIK

SEQ ID NO: 53 - protein, 107 aa, Mus musculus
DIQMTQSPASLSVSVGETVTITCRASVNIYSNLAWYQQRQGKSPQLLVYAATNLAEGVPSRFSGSGSGTQYSLKINSLQSEDFGSYYCQHFWVTPYTFGGGTKLEIK

SEQ ID NO: 54 - protein, 118 aa, Artificial Sequence
  Synthetic Construct
EVQLVESGGGLVQPGGSLRLSCAASGFTFSSYSMSWVRQAPGKGLEWVAYISNGGSTTYYPGTVEGRFTISRDNSKNTLYLQMNSLRAEDTAVYYCARHRTNWDFDYWGQGTLVTVSS

SEQ ID NO: 55 - protein, 118 aa, Artificial Sequence
  Synthetic Construct
EVQLVESGGGLVQPGGSLRLSCAASGFTFSSYSMSWVRQAPGKGLEWVAYISNGGSTTDYPGTVEGRFTISRDNSKNTLYLQMNSLRAEDTAVYYCARHRTNWDFDYWGQGTLVTVSS

SEQ ID NO: 56 - protein, 118 aa, Artificial Sequence
  Synthetic Construct
EVQLVESGGGLVQPGGSLRLSCAASGFTFSFYSMSWVRQAPGKGLEWVARISNGGSTTDYPGTVEGRFTISRDNSKNTLYLQMNSLRAEDTAVYYCARHRTNWDFDYWGQGTLVTVSS

SEQ ID NO: 57 - protein, 118 aa, Artificial Sequence
  Synthetic Construct
EVQLVESGGGLVQPGGSLRLSCAASGFTFSSYLMSWVRQAPGKGLEWVARISNGGSTTDYPGTVEGRFTISRDNSKNTLYLQMNSLRAEDTAVYYCARHRTNWDFDYWGQGTLVTVSS

SEQ ID NO: 58 - protein, 118 aa, Artificial Sequence
  Synthetic Construct
EVQLVESGGGLVQPGGSLRLSCAASGFTFSSYKMSWVRQAPGKGLEWVAVISNGGSTTDYPGTVEGRFTISRDNSKNTLYLQMNSLRAEDTAVYYCARHRTNWDFDYWGQGTLVTVSS

SEQ ID NO: 59 - protein, 118 aa, Artificial Sequence
  Synthetic Construct
EVQLVESGGGLVQPGGSLRLSCAASGFTFSFYLMSWVRQAPGKGLEWVARISNGGSTTDYPGTVEGRFTISRDNSKNTLYLQMNSLRAEDTAVYYCARHRTNWDFDYWGQGTLVTVSS

SEQ ID NO: 60 - protein, 118 aa, Artificial Sequence
  Synthetic Construct
EVQLVESGGGLVQPGGSLRLSCAASGFTFSSYLMSWVRQAPGKGLEWVAYISNGGSTTDYPGTVEGRFTISRDNSKNTLYLQMNSLRAEDTAVYYCARHRTNWDFDYWGQGTLVTVSS

SEQ ID NO: 61 - protein, 118 aa, Mus musculus
EVKLVESGGGLVQPGGSLKLSCAASGFTFSSYSMSWVRQTPEKRLEWVAYISNGGSTTYYPGTVEGRFTISRDNAKNTLFLQMSSLRSKDTAMYYCARHRTNWDFDYWGQGTTLTVSS

SEQ ID NO: 62 - protein, 122 aa, Mus musculus
QVQLQQSGTGLVRPGTSVRVSCKASGYAFITYLIEWIKQRPGQGLEWIGVIHPGSGNSHYNEKFKGKATLTADTSSSTAYMQLSSLTSGDSAVYFCARSGSSSFDYYAMDFWGQGTSVTVSS

SEQ ID NO: 63 - protein, 122 aa, Mus musculus
QVQLQQSGADLVRPGTSVKVSCKASGYAFTNYLIDWVKQRPGQGLEWIGAIHPGSGRTHYNEKFKGKATLTADKSSSAAYMQISSLTSDDSAVFFCARSGGSSFDYYAMDYWGQGTSVTVSS

SEQ ID NO: 64 - protein, 122 aa, Artificial Sequence
  Synthetic Construct
EVQLVQSGAEVKKPGASVKVSCKASGYAFITYLIEWVRQAPGQGLEWIGVIHPGSGNSHYNEKFKGRATLTADTSTSTAYLELSSLRSEDTAVYYCARSGSSSFDYYAMDFWGQGTLVTVSS

SEQ ID NO: 65 - protein, 117 aa, Mus musculus
QVQLKESGPGLVAPSQSLSITCTVSGFSLTGYGVNWVRQPPGKGLEWLGMIWDDGTTDFDSALRSRLSISKDNSKSQVFLKMNSLQTDDTARYFCARGDYGYAMDYWGQGTSVTVSS

SEQ ID NO: 66 - protein, 214 aa, Artificial Sequence
  Synthetic Construct
DIQMTQSPSSLSASVGDRVTITCRASENIYSNLAWYQQKPGKAPKLVVYAATQLADGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 214 aa, Artificial Sequence
  Synthetic Construct
DIQMTQSPSSLSASVGDRVTITCRASENIYWNLAWYQQKPGKAPKLVVYAATQLADGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 214 aa, Artificial Sequence
  Synthetic Construct
DIQMTQSPSSLSASVGDRVTITCRASENIYYNLAWYQQKPGKAPKLVVYAATQLADGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 214 aa, Artificial Sequence
  Synthetic Construct
DIQMTQSPSSLSASVGDRVTITCRASENIYYNLAWYQQKPGKAPKLLIYAATQLAEGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 214 aa, Artificial Sequence
  Synthetic Construct
DIQMTQSPSSLSASVGDRVTITCRASENIYYNLAWYQQKPGKAPKLLIYAATQLADGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 214 aa, Artificial Sequence
  Synthetic Construct
DIQMTQSPSSLSASVGDRVTITCRASENIYWNLAWYQQKPGKAPKLLIYAATQLADGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 214 aa, Artificial Sequence
  Synthetic Construct
DIQMTQSPSSLSASVGDRVTITCRASENIYYNLAWYQQKPGKAPKLLIYAATQLASGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 214 aa, Artificial Sequence
  Synthetic Construct
DIQMTQSPSSLSASVGDRVTITCRASENIYWNLAWYQQKPGKAPKLLIYAATQLASGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 214 aa, Artificial Sequence
  Synthetic Construct
DIQMTQSPSSLSASVGDRVTITCRASENIYSNLAWYQQKPGKAPKLLIYAATQLADGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 214 aa, Artificial Sequence
  Synthetic Construct
DIQMTQSPSSLSASVGDRVTITCRASENIYSNLAWYQQKPGKAPKLLIYAATQLAEGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 214 aa, Artificial Sequence
  Synthetic Construct
DIQMTQSPSSLSASVGDRVTITCRASENIYSNLAWYQQKPGKAPKLLIYAATQLASGVPSRFSGSGSGTDYTLTISSLQPEDFATYYCQHFWITPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 448 aa, Artificial Sequence
  Synthetic Construct
EVQLVESGGGLVQPGGSLRLSCAASGFTFSSYSMSWVRQAPGKGLEWVAYISNGGSTTYYPGTVEGRFTISRDNSKNTLYLQMNSLRAEDTAVYYCARHRTNWDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 448 aa, Artificial Sequence
  Synthetic Construct
EVQLVESGGGLVQPGGSLRLSCAASGFTFSSYSMSWVRQAPGKGLEWVAYISNGGSTTDYPGTVEGRFTISRDNSKNTLYLQMNSLRAEDTAVYYCARHRTNWDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 448 aa, Artificial Sequence
  Synthetic Construct
EVQLVESGGGLVQPGGSLRLSCAASGFTFSFYSMSWVRQAPGKGLEWVARISNGGSTTDYPGTVEGRFTISRDNSKNTLYLQMNSLRAEDTAVYYCARHRTNWDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 448 aa, Artificial Sequence
  Synthetic Construct
EVQLVESGGGLVQPGGSLRLSCAASGFTFSSYLMSWVRQAPGKGLEWVARISNGGSTTDYPGTVEGRFTISRDNSKNTLYLQMNSLRAEDTAVYYCARHRTNWDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 448 aa, Artificial Sequence
  Synthetic Construct
EVQLVESGGGLVQPGGSLRLSCAASGFTFSSYKMSWVRQAPGKGLEWVAVISNGGSTTDYPGTVEGRFTISRDNSKNTLYLQMNSLRAEDTAVYYCARHRTNWDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 448 aa, Artificial Sequence
  Synthetic Construct
EVQLVESGGGLVQPGGSLRLSCAASGFTFSFYLMSWVRQAPGKGLEWVARISNGGSTTDYPGTVEGRFTISRDNSKNTLYLQMNSLRAEDTAVYYCARHRTNWDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 448 aa, Artificial Sequence
  Synthetic Construct
EVQLVESGGGLVQPGGSLRLSCAASGFTFSSYLMSWVRQAPGKGLEWVAYISNGGSTTDYPGTVEGRFTISRDNSKNTLYLQMNSLRAEDTAVYYCARHRTNWDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 395 aa, Homo sapiens
MSANATLKPLCPILEQMSRLQSHSNTSIRYIDHAAVLLHGLASLLGLVENGVILFVVGCRMRQTVVTTWVLHLALSDLLASASLPFFTYFLAVGHSWELGTTFCKLHSSIFFLNMFASGFLLSAISLDRCLQVVRPVWAQNHRTVAAAHKVCLVLWALAVLNTVPYFVFRDTISRLDGRIMCYYNVLLLNPGPDRDATCNSRQAALAVSKFLLAFLVPLAIIASSHAAVSLRLQHRGRRRPGRFVRLVAAVVAAFALCWGPYHVFSLLEARAHANPGLRPLVWRGLPFVTSLAFFNSVANPVLYVLTCPDMLRKLRRSLRTVLESVLVDDSELGGAGSSRRRRTSSTARSASPLALCSRPEEPRGPARLLGWLLGSCAASPQTGPLNRALSSTSS

SEQ ID NO: 85 - protein, 396 aa, Macaca mulatta
MSANATLKPLCPILEEMSHLRSHSNTSIRYIDHATVLLHGLASLLGLVENGVILFVVGCRMRQTVVTTWVLHLALSDLLASASLPFFTYFLAVGHSWELGTTFCKLHSSIFFLNMFASGFLLSAISLDRCLQVVWPVWAQNHRTVAAAHKVCLVLWALAVLNTVPYFVFRDTISRLDGRIMCYYNVLLLNPGPDRDATCNSRQAALAVSKFLLAFLVPLAIIASSHAAVSLRLQHRGRRRPGRFVRLVAAVVAAFALCWGPYHVFSLLEARAHANPGLRPLVWRGLPFVTSLAFFNSVANPVLYVLTCPDMLRKLRRSLRTVLESVLVDDSELGGAGSSRRRRRTPSTARSASSLALSSRPEERRGPARLFGWLLGGCAASPQRGPLNRALSSTSS

SEQ ID NO: 86 - protein, 396 aa, Artificial Sequence
  Synthetic Construct
MSANATLKPLCPILEEMSHLRSHSNTSIRYIDHATVLLHGLASLLGLVENGVILFVVGCRMRQTVVTTWVLHLALSDLLASASLPFFTYFLAVGHSWELGTTFCKLHSSIFFLNMFASGFLLSAISLDRCLQVVWPVWAQNHRTVAAAHKVCLVLWALAVLNTVPYFVFRDTISRLDGRIMCYYNVLLLNPGSDRDATCNSRQAALAVSKFLLAFLVPLAIIASSHAAVSLRLQHRGRRRPGRFVRLVAAVVAAFALCWGPYHVFSLLEARAHANRGLRPLVWRGLPFVTSLAFFNSVANPVLYVLTCPDMLRKLRRSLRTVLESVLVDDSELGGAGSSRRRRRTPSTARSASSLALSSHPEERRGPARLFGWLLGGCAASPQRGPLNRALSSTSS

SEQ ID NO: 87 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 88 - protein, 779 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 89 - protein, 781 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEDTPVRKLQANKKVQGSRRRLREGRSQ

SEQ ID NO: 90 - protein, 751 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEDTPVRKLQANKKVQGSRRRLREGRSQ

SEQ ID NO: 91 - protein, 695 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYSPEIKYTRISTGGGETEETLKKLLQE

(19) United States
(12) Patent Application Publication
(10) Pub. No.: US 2016/0090422 A1
(43) Pub. Date: Mar. 31, 2016

(54) ANTI-CRTH2 ANTIBODIES AND METHODS OF USE

(71) Applicant: GENENTECH, INC., South San Francisco, CA (US)

(72) Inventors: Karin REIF, South San Francisco, CA (US); Isidro HOTZEL, South San Francisco, CA (US); Jo-Anne S. HONGO, South San Francisco, CA (US); Tao HUANG, South San Francisco, CA (US); Yonglei SHANG, South San Francisco, CA (US); Meredith HAZEN, South San Francisco, CA (US)

(21) Appl. No.: 14/853,841

(22) Filed: Sep. 14, 2015

Related U.S. Application Data
(63) Continuation of application No. PCT/US2014/029455, filed on Mar. 14, 2014.
(60) Provisional application No. 61/786,370, filed on Mar. 15, 2013.

(51) Int. Cl. C07K 16/28

(52) U.S. Cl. CPC C07K 16/2896; C07K 2317/24; C07K 2317/732; C07K 2317/734; C07K 2317/41; C07K 2317/14; C07K 2317/52; C07K 2317/31; C07K 2317/76; C07K 2317/33; C07K 2317/92; C07K 2317/56; A61K 2039/505

(57) ABSTRACT

The invention provides anti-CRTh2 antibodies and methods of using the same.

### CROSS REFERENCES TO RELATED APPLICATIONS

This application is a continuation of International Patent Application No. PCT/US2014/029455, filed Mar. 14, 2014, which claims the priority benefit of U.S. Provisional-Application Ser. No. 61/786,370, filed Mar. 15, 2013, each of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392017330SEQLIST.TXT, date recorded: Sep. 14, 2015, size: 115 KB).

### FIELD OF THE INVENTION

The present invention relates to anti-CRTh2 antibodies and methods of using the same.

### BACKGROUND

Chemoattractant receptor-homologous molecule expressed on T helper 2 cells (CRTh2) is a member of the G-protein coupled receptor (GPCR) family CRTh2 mediates chemotaxis of eosinophils, basophils, and T helper type 2 (Th2) cells in response to prostaglandin D2 (PGD2). These cell types, specifically Th2 cells, have been considered to contribute to the pathogenesis of allergic diseases, such as asthma. It has been shown that CRTh2 inhibition leads to attenuated airway hyperreactivity and inflammation in animal models. Lukacs, et al.; Am. J. Phsiol. Lung Cell. Mol. Physiol. 295:L767-779, 2008. For example, ramatroban, a dual thrombroxane A2 receptor and CRTh2 receptor antagonist, suppresses eosinophil chemotaxis in vitro and in vivo and is approved for the treatment of allergic rhinitis in Japan. Bosnjak, B, et. al, Respiratory Research 12:114, 2011. Numerous other CRTh2 antagonists, such as 4-aminotetrahyrochinoline derivatives or indoleacetic acid derivatives, are currently under development. Pettipher; Br. J. Pharmacol. 153 (Suppl 1):5191-199, 2008; Royer et al.; Eur. J. Clin. Invest. 38:663-671, 2008; Stebbins et al.; Eur. J. Pharmacol. 638:142-149, 2010.

### BRIEF SUMMARY

The invention provides anti-CRTh2 antibodies and methods of using the same.

In one aspect, provided herein is an isolated antibody that binds human CRTh2 and depletes CRTh2 expressing cells when a therapeutically effective amount is administered to a human subject. In some embodiments, the anti-CRTh2 antibody is an engineered antibody. In some embodiments, the anti-CRTh2 antibody is produced by a recombinant method (e.g., by a host cell transfected or transformed with a nucleic acid or nucleic acids encoding the antibody in vitro (for example, in cell culture)). In some embodiments, the host cell is a prokaryotic cell (e.g., a bacterial cell) or a eukaryotic cell (e.g., a CHO cell, a lymphoid cell).

In some embodiments, the antibody depletes one or more of the following types of CRTh2 expressing cells: Th2 cells, mast cells, eosinophils, basophils, or innate type 2 (IT2) cells. In some embodiments, the antibody has been engineered to improve ADCC and/or CDC activity. In some embodiments, the antibody has been engineered to improve ADCC and/or reduce CDC activity. In some embodiments, the antibody is afucosylated. In some embodiments, the antibody is produced in a cell line having a alpha1,6-fucosyltransferase (Fut8) knockout. In some embodiments, the antibody is produced in a cell line overexpressing β1,4-N-acetylglycosaminyltransferase III (GnT-III). In some embodiments, the cell line additionally overexpresses Golgi α-mannosidase II (ManII). In some embodiments, the antibody comprises at least one amino acid substitution in the Fc region that improves ADCC and/or CDC activity. In some embodiments, the amino acid substitutions are S298A/E333A/K334A.

In some embodiments, the antibody is a naked antibody. In some embodiments, the antibody is chimeric. In some embodiments, the antibody is humanized. In some embodiments, the antibody is human. In some embodiments, the antibody is a bispecific antibody. In some embodiments, the antibody is an IgG1 antibody.

In some embodiments, the antibody binds to CRTh2 of a non-human primate. In some embodiments, the antibody binds to rhesus and/or cynomologous CRTh2.

In some embodiments, the antibody competitively inhibits binding of at least one of the following antibodies: 19A2, 8B1, 31A5, 3C12, and any of the humanized antibodies described herein to human CRTh2. In some embodiments, an ELISA assay is used to determine competitive binding. In some embodiments, the antibody binds to an epitope of human CRTh2 that is the same as or overlaps with the CRTh2 epitope bound by at least one of the following anti-CRTh2 antibodies: 19A2, 8B1, 31A5, 3C12, and any of the humanized antibodies described herein. In some embodiments, the antibody comprises the six hypervariable regions (HVRs) from one of the following anti-CRTh2 antibodies: 19A2, 8B1, 31A5, 3C12, and any of the humanized antibodies described herein.

In some embodiments, the antibody further blocks CRTh2 signaling. In some embodiments, the antibody prevents recruitment of CRTh2 expressing cells in response to prostaglandin D2. In some embodiments, the antibody blocks Ca2⁺ flux in CRTh2 expressing cells. In some embodiments, the antibody binds human CRTh2 with a Kd value of about 100 nM or less.

In some embodiments, the antibody comprises HVR-H3 comprising the amino acid sequence of SEQ ID NO:6, HVR-L3 comprising the amino acid sequence of SEQ ID NO:3, and HVR-H2 comprising X₁ISNGGSTTX₂YPGTVEG (SEQ ID NO:5), wherein X₁ is Y or R, and X₂ is Y or D. In some embodiments, the antibody comprises HVR-H3 comprising the amino acid sequence of SEQ ID NO:35 or 36, HVR-L3 comprising the amino acid sequence of SEQ ID NO:27, and HVR-H2 comprising the amino acid sequence of SEQ ID NO:32 or 33. In some embodiments, the antibody comprises HVR-H3 comprising the amino acid sequence of SEQ ID NO:37, HVR-L3 comprising the amino acid sequence of SEQ ID NO:28, and HVR-H2 comprising the amino acid sequence of SEQ ID NO:34.

In another aspect, provided herein is an isolated anti-CRTh2 antibody comprising a light chain and heavy chain variable region, wherein the light chain and heavy chain variable region comprises six hypervariable region (HVR) sequences: (i) HVR-L1 comprising RASENIYXNLA (SEQ ID NO:1), wherein X is S, W, or Y; (ii) HVR-L2 comprising AATQLAX (SEQ ID NO:2), wherein X is D, E, or S; (iii) HVR-L3 comprising QHFWITPWT (SEQ ID NO:3); (iv) HVR-H1 comprising X₁YX₂MS (SEQ ID NO:4), wherein X₁ is S or F, and X₂ is S, L, or K; (v) HVR-H2 comprising X₁ISNGGSTTX₂YPGTVEG (SEQ ID NO:5), wherein X₁ is Y or R, and X₂ is Y or D; and (vi) HVR-H3 comprising HRTNWDFDY (SEQ ID NO:6).

In another aspect, provided herein is an isolated anti-CRTh2 antibody comprising a light chain and heavy chain variable region, wherein the light variable region comprises HVR-L1 comprising the amino acid sequence of SEQ ID NO:7, 8, or 9, HVR-L2 comprising the amino acid sequence of SEQ ID NO:10, 11, or 12, and HVR-L3 comprising the amino acid sequence of SEQ ID NO:3. In some embodiments, the antibody further comprises the heavy chain variable region comprising HVR-H1 comprising the amino acid sequence of SEQ ID NO:13, 14, 15, 16, or 17, HVR-H2 comprising the amino acid sequence of SEQ ID NO:18, 19, 20, or 21, and HVR-H3 comprising amino acid sequence of SEQ ID NO:6.

In another aspect, provided herein is an isolated anti-CRTh2 antibody comprising a light chain and heavy chain variable region, comprising the heavy chain variable region comprising HVR-H1 comprising the amino acid sequence of SEQ ID NO:13, 14, 15, 16, or 17, HVR-H2 comprising the amino acid sequence of SEQ ID NO:18, 19, 20, or 21, and HVR-H3 comprising amino acid sequence of SEQ ID NO:6.

In some embodiments, the antibody comprises: (i) HVR-L1 comprising the amino acid sequence of SEQ ID NO:8; (ii) HVR-L2 comprising the amino acid sequence of SEQ ID NO:10; (iii) HVR-L3 comprising the amino acid sequence of SEQ ID NO:3; (iv) HVR-H1 comprising the amino acid sequence of SEQ ID NO:13; (v) HVR-H2 comprising the amino acid sequence of SEQ ID NO:19; and (vi) HVR-H3 comprising the amino acid sequence of SEQ ID NO:6.

In some embodiments, the antibody comprises: (i) HVR-L1 comprising the amino acid sequence of SEQ ID NO:9; (ii) HVR-L2 comprising the amino acid sequence of SEQ ID NO:11; (iii) HVR-L3 comprising the amino acid sequence of SEQ ID NO:3; (iv) HVR-H1 comprising the amino acid sequence of SEQ ID NO:15; (v) HVR-H2 comprising the amino acid sequence of SEQ ID NO:20; and (vi) HVR-H3 comprising the amino acid sequence of SEQ ID NO:6.

In another aspect, provided herein is an isolated anti-CRTh2 antibody comprising a light chain and heavy chain variable region, wherein the light variable region comprises HVR-L1 comprising the amino acid sequence of SEQ ID NO:9, HVR-L2 comprising the amino acid sequence of SEQ ID NO:10, and HVR-L3 comprising the amino acid sequence of SEQ ID NO:3. In another aspect, provided herein is an isolated anti-CRTh2 antibody comprising a light chain and heavy chain variable region, comprising the heavy chain variable region comprising HVR-H1 comprising the amino acid sequence of SEQ ID NO:15, HVR-H2 comprising the amino acid sequence of SEQ ID NO:20, and HVR-H3 comprising amino acid sequence of SEQ ID NO:6. In some embodiments, the antibody comprises: (i) HVR-L1 comprising the amino acid sequence of SEQ ID NO:9; (ii) HVR-L2 comprising the amino acid sequence of SEQ ID NO:10; (iii) HVR-L3 comprising the amino acid sequence of SEQ ID NO:3; (iv) HVR-H1 comprising the amino acid sequence of SEQ ID NO:15; (v) HVR-H2 comprising the amino acid sequence of SEQ ID NO:20; and (vi) HVR-H3 comprising the amino acid sequence of SEQ ID NO:6.

In another aspect, provided herein is an isolated anti-CRTh2 antibody comprising a light chain and heavy chain variable region, wherein the antibody comprise a VL sequence selected from the group consisting of SEQ ID NOS:38-53. In some embodiments, the antibody further comprises a VH sequence selected from the group consisting of SEQ ID NOS:54-65. In another aspect, provided herein is an isolated anti-CRTh2 antibody comprising a light chain and heavy chain variable region, wherein the antibody comprise a VH sequence selected from the group consisting of SEQ ID NOS:54-65. In some embodiments, provided herein is an isolated anti-CRTh2 antibody comprising a light chain variable region comprising a VL sequence selected from the group consisting of SEQ ID NOS:38-48 and a heavy chain variable region comprising a VH sequence selected from the group consisting of SEQ ID NOS:54-60.

In some embodiments, the antibody comprises a VL sequence of SEQ ID NO:40 and a VH sequence of SEQ ID NO:57. In some embodiments, the antibody comprises a VL sequence of SEQ ID NO:39 and a VH sequence of SEQ ID NO:55. In some embodiments, the antibody comprises a VL sequence of SEQ ID NO:41 and a VH sequence of SEQ ID NO:57.

In some embodiments, the antibody is monoclonal antibody. In some embodiments, the antibody is a humanized or chimeric antibody. In some embodiments, at least a portion of the framework sequence of the antibody is a human consensus framework sequence. In some embodiments, the antibody is an antibody fragment selected from a Fab, Fab′-SH, Fv, scFc or (Fab′)₂ fragment.

In another aspect, provided herein is an isolated nucleic acid encoding any of the antibody described herein. In another aspect, provided herein is a host cell comprising the nucleic acid described herein. In another aspect, provided herein is a method of producing an antibody comprising culturing the host cell so that the antibody is produced. In some embodiments, the method further comprises recovering the antibody produced by the host cell.

In another aspect, provided herein is an immunoconjugate comprising any of the antibody described herein and a cytotoxic agent. In some embodiments, the immunoconjugate is in a pharmaceutical composition. The immunoconjugate may be used in any of the methods described herein.

In another aspect, provided herein is a pharmaceutical composition comprising any of the anti-CRTh2 antibody described herein and a pharmaceutically acceptable carrier.

In another aspect, provided herein is a method for treating asthma comprising administering an effective amount of an anti-CRTh2 antibody to a subject, wherein the antibody depletes CRTh2 expressing cells in the subject.

In some embodiments, the antibody depletes one or more of the following types of CRTh2 expressing cells: Th2 cells, mast cells, eosinophils, basophils, or innate type 2 (IT2) cells. In some embodiments, the anti-CRTh2 antibody depletes CRTh2 expressing cells from lung tissue. In some embodiments, the anti-CRTh2 antibody depletes CRTh2 expressing cells from bronchoalveolar lavage fluid. In some embodiments, the anti-CRTh2 antibody depletes at least 50% of at least one type of CRTh2 expressing cell from the lung compared to the baseline before administering the antibody. In some embodiments, the anti-CRTh2 antibody depletes at least 80% of at least one type of CRTh2 expressing cell from the lung compared to the baseline before administering the antibody. In some embodiments, the anti-CRTh2 antibody depletes at least 90% of at least one type of CRTh2 expressing cell from the lung compared to the baseline before administering the antibody. In some embodiments, the subject is suffering from pauci granulocytic asthma. In some embodiments, the level of one or more cytokines is reduced in the subject following administration of the anti-CRTh2 antibody. In some embodiments, the level of one or more cytokines produced by at least one of the following cell types is reduced: Th2 cells, mast cells, eosinophils, basophils, or innate type 2 (IT2) cells. In some embodiments, the level of one or more of IL-4, IL-5, IL-9, IL-13, IL-17, histamines or leukotrienes is reduced in the subject. In some embodiments, the subject is suffering from asthma that is not adequately controlled by an inhaled corticosteroid, a short acting β2 agonist, a long acting β2 agonist, or a combination thereof. In some embodiments, the subject is a human. In some embodiments, the anti-CRTh2 antibody is an antibody described herein.

In another aspect, provided herein is a method for treating a disorder mediated by CRTh2 expressing cells comprising administering an effective amount of an anti-CRTh2 antibody to a subject, wherein the antibody depletes CRTh2 expressing cells in the subject.

In some embodiments, the disorder is selected from the group consisting of: asthma, pauci granulocytic asthma, atopic dermatitis, allergic rhinitis, acute or chronic airway hypersensitivity, hypereosinophilic syndrome, eosinophilic esophagitis, Churg-Strauss syndrome, idiopathic pulmonary fibrosis, inflammation associated with a cytokine, inflammation associated with CRTh2 expressing cells, malignancy associated with CRTh2 expressing cells, chronic idiopathic urticaria, chronic spontaneous urticaria, physical urticaria, cold urticaria, pressure-urticaria, bullous pemphigoid, nasal polyposis, food allergy, and allergic bronchopulmonary aspergillosis (ABPA). In some embodiments, the anti-CRTh2 antibody is an antibody described herein.

In another aspect, provided herein is a method for reducing the level of a cytokine in a subject comprising administering an effective amount of an anti-CRTh2 antibody to a subject, wherein the antibody depletes CRTh2 expressing cells in the subject. In some embodiments, the level of one or more IL-4, IL-5, IL-9, IL-13, IL-17, histamines or leukotrienes is reduced in the subject. In some embodiments, the anti-CRTh2 antibody is an antibody described herein.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### DESCRIPTION OF THE FIGURES

FIG. 1 shows that CRTh2 is expressed on human ‘Th2’ biology cells. CRTh2 expression was assessed by flow cytometry using anti-human CRTh2 Ab (BM16) on human PBMCs populations or cultured human cells as indicated

FIG. 2 shows that CRTh2+ memory CD4+ T cells produce more than 95% of memory CD4+ T cell Th2 cytokines (Il-4, IL-5, IL13 and IL-9) when compared to CRTh2-memory CD4+ T cells. CRTh2+CD45RO+ and CRTh2-CD45RO+ memory CD4+ T cells were isolated by flow cytometry from human PBMC and stimulated with anti-CD3 and anti-CD28 antibodies for 48 hrs at 37° C. Supernatants were collected and subjected to cytokine quantitation as indicated by Luminex.

FIGS. 3A-F show reactivity of mouse or humanized anti-CRTh2 antibodies by flow cytometry with CRTh2 expressed on cell lines or with primary basophils and eosinophils. FIG. 3A shows reactivity by flow cytometry of mouse anti-CRTh2 hybridoma antibodies (clones 19A2, 8B1, 31A5 and 3C12) compared to control Ab (at 20 ug/ml, tinted histogram) with human, rhesus monkey or cynomolgus monkey CRTh2 expressed on 293 cells, as well as with wild-type 293 cells that do not express CRTh2. Primary antibody concentrations used were 20 ug/ml (black line), 2 ug/ml (grey line) and 0.2 ug/ml (light grey line). FIG. 3B shows reactivity by flow cytometry of mouse anti-CRTh2 antibodies (19A2 and 8B1 cloned with mIgG2a) compared to isotype control Ab (tinted histogram) with human, rhesus monkey or cynomolgus monkey amino terminal flag-tagged CRTh2 expressed on 300.19 cells, as well as with wild-type 300.19 cells that do not express CRTh2. Primary antibody concentration used were 1 ug/ml (human, cyno; black line) or 5 ug/ml (rhesus, wild type; black line) and 0.5 ug/ml (rhesus, wild type; grey line). The anti-Flag Ab was used at 0.7 ug/ml. FIG. 3C shows reactivity by flow cytometry of mouse anti-human CRTh2 antibodies (19A2, 8B1, 31A5, 3C12) to basophils and eosinophils on human PBMCs. PBMC were incubated with anti-CRTh2 antibodies at 5 ug/ml (black line), 0.5 ug/ml (grey line) or with isotype control Ab at 5 ug/ml (light grey line), 0.5 ug/ml (tinted histogram) followed by fluorescent-labeled secondary anti-mouse IgG, anti-CD16, anti-HLADR, and anti-CD123. FIG. 3D and FIG. 3E show reactivity of humanized h19A2.v1 and engineered humanized h19A2.v12 anti-CRTh2 antibodies with amino terminal gD-tagged or flag-tagged human, rhesus or cynomolgus CRTh2 expressed on 293 cells (FIG. 3D) or 300.19 cells (FIG. 3E), respectively, compared to respective wild-type 293 or 300.19 cells, that do not express CRTh2. Primary anti-CRTh2 Ab concentrations used were: 10 ug/ml (black line), 1 ug/ml (grey line) and 0.1 ug/ml (light grey line); isotype control Ab (2H7, tinted histogram) was used at 10 ug/ml, anti-gD antibody was used at 2 ug/ml and anti-Flag Ab was used at 0.7 ug/ml. FIG. 3F shows FACS binding of anti-CRTh2 antibodies h19A2.v1 and h19A2.v12 at 10 ug/ml (black line) to primary human, cyno and rhesus basophils as well as to primary human eosinophils from peripheral blood compared to isotype control Ab (tinted histogram).

FIGS. 4A-B show Scatchard analysis of the binding affinities of anti-CRTh2 antibodies (mIgG or hFab) to surface expressed CRTh2 on 293 cells or 300.19 cells. FIG. 4A shows the radioligand cell binding assay of mouse anti-CRTh2 whole antibodies 19A2 and 8B1 to human CRTh2 expressed on 293 cells or 300.19 cells as indicated. FIG. 4B shows the radioligand cell binding assay of humanized h19A2.v12 or h19A2.v60 Fab fragments to human or cynomolgus CRTh2 expressed on 293 cells. The dissociation constant (K_{D}) for anti-CRTh2 Abs is indicated in the graphs. Bound/Total indicates the ratio of concentrations of bound ¹²⁵I-labeled antibody and total antibody; total indicates concentrations of ¹²⁵I-labeled and unlabeled antibody.

FIG. 5 shows that anti-CRTh2 antibodies 8B1 and 3C12 prevent PGD2 induced calcium mobilization. Calcium flux of the Th2 cell subset (CD4+CCR4+CCR6-CXCR3-) from in vitro polarized Th2 cells in response to PGD2 stimulation was monitored by flow cytometry in the presence of anti-CRTh2 or isotype control antibodies. The CRTh2 receptor antagonist CAY10471 is included as a positive control.

FIGS. 6A-B show the design and characterization of human CRTh2 BAC transgenic mice. FIG. 6A depicts the 171 kb genomic region containing the human CRTh2 gene on chromosome 11 that was introduced into C57BL/6 mice to generate hCRTh2 BAC transgenic mice. FIG. 6B shows human CRTh2 expression (antibody BM16) by flow cytometry on blood basophil (CD123+FceRI+), blood eosinophils (CCR3+), peritoneal mast cell (FceRI+CD117+), popiteal lymph node CD4+CD44hi T cells (induced by the Th2 polarizing agent papain), and mesentery lymph node innate T helper type 2 cells (Lin-CD117+ boosted by hydrodynamic tail vein injection of mouse IL-17E plasmid) in hCRTh2.Bac.Tg line 85. For comparison flow cytometry analyses of human CRTh2 expression on human cells is shown. Basophils, eosinophils and IT2 cells were stained from PBMC, mast cells from human bone marrow-derived mast cells, and Th2 cells (CCR4+CXCR3-) were differentiated under Th2 polarizing conditions from CD4+ T cells isolated from human PBMC.

FIGS. 7A-B show that anti-CRTh2 antibodies deplete blood basophils and eosinophils in vivo in human CRTh2.Bac.Tg mice. Baseline numbers of CRTh2+ basophils (CD123+FceRI+) and eosinophils (CCR3+) was determined by flow cytometry from blood on day-4 (FIG. 7A) or 4 hours (FIG. 7B) before treatment with anti-CRTh2 Abs (19A2, 3C12 or 8B1 as indicated). Human CRTh2.Bac.Tg mice were treated with anti-CRTh2 or isotype control antibodies at 200 ug/mouse i.v. (FIG. 7A) or 150 ug/mouse i.v. (FIG. 7B). Blood basophil and eosinophil depletion was assessed by flow cytometry on day 3, day 6 or day 7 as indicated. Percent depletion by anti-CRTh2 as compared to anti-ragweed isotype control antibodies is indicated in FIG. 7B.

FIGS. 8A-B show that anti-CRTh2 antibody 19A2 treatment depleted innate immune cells and reduced Th2 bronchoalveolar lavage (BAL) cytokine production in a TNP-OVA induced chronic asthma model in hCRTh2.Bac.Tg mice. FIG. 8A shows basophil, eosinophil and mast cell numbers that were assessed in lung tissue by flow cytometry and in BAL by differential cell count combined with flow cytometry (FIG. 8A). Percent depletion by anti-CRTh2 as compared to anti-ragweed isotype control antibodies is indicated in the graphs. FIG. 8B shows the concentrations of IL-4 and IL-13 determined by ELISA in BAL. Percent reduction by anti-CRTh2 treatment compared to isotype control antibodies is indicated in the graphs.

FIGS. 9A-B show that anti-CRTh2 antibody 19A2 depletes human IL-4 producing Th2 cells in SCID mice or innate type helper 2 (IT2) cells in human CRTh2.Bac.Tg mice. FIG. 9A: In vitro polarized human Th2 cells from PBMC were transferred into SCID mice and further polarized for 7 days in vivo by injecting rhIL-4 plus anti-IFN-g and anti-IL-12 mAbs in the presence of afucosylated anti-CRTh2 19A2 antibodies or isotype control antibodies. After 7 days, the percentage of IL-4 or IFN-g producing CD4 T cells was determined. For this purpose, splenocytes were harvested and stimulated ex vivo with PdBu (50 ng/mL) and Ionomycin (500 ng/mL) for 4.5 hrs with brefeldin A (BFA) being added during the last 3 hours of stimulation. Cells were surface stained with anti-hCD4 and lineage cells were stained with anti-mCD45, anti-mTer119, and anti-hCD19; cells were fixed and stained with anti-hIFNg and anti-hIL-4 to detect cytokine positive cells. FIG. 9B: Human CRTh2. Bac.Tg mice were injected with 50 ug mouse IL-17E encoding plasmid followed by anti-CRTh2 or isotype control Abs. On day 3 after treatment, the percentage and total number of IT2 cells was determined in mesenteric lymph nodes by flow cytometry. Percent depletion by anti-CRTh2 as compared to anti-ragweed isotype control antibodies is indicated in the graphs.

FIG. 10 shows the amino acid sequence of the light chain (SEQ ID NO:49) and heavy chain (SEQ ID NO:61) variable regions of murine anti-CRTh2 antibody 19A2. Kabat CDR, Chothia CDR and Contact CDR sequences of the heavy and light chain are provided.

FIGS. 11A-B show the amino acid sequence alignment of light chain and heavy chain variable regions of humanized anti-CRTh2 antibodies derived from antibody 19A2. FIG. 11A shows the light chain variable region sequence alignment. Light chain Kabat CDR, Chothia CDR, and Contact CDR sequences of each antibody are provided (hu19A2.v1 (SEQ ID NO:38), hu19A2.v12 (SEQ ID NO:39), hu19A2.v46 (SEQ ID NO:39), hu19A2.v52 (SEQ ID NO:40), hu19A2.v58 (SEQ ID NO:42), hu19A2.v60 (SEQ ID NO:41), hu19A2.v61 (SEQ ID NO:42), hu19A2.v62 (SEQ ID NO:41), hu19A2.v63 (SEQ ID NO:43), hu19A2.v64 (SEQ ID NO:42), hu19A2.v65 (SEQ ID NO:43), hu19A2.v66 (SEQ ID NO:44), hu19A2.v67 (SEQ ID NO:45), hu19A2.v68 (SEQ ID NO:44), hu19A2.v69 (SEQ ID NO:45), hu19A2.v70 (SEQ ID NO:46), hu19A2.v71 (SEQ ID NO:47), hu19A2.v72 (SEQ ID NO:48)). FIG. 11B shows the heavy chain variable region sequence alignment. Heavy chain Kabat CDR, Chothia CDR, and Contact CDR sequences of each antibody are provided (hu19A2.v1 (SEQ ID NO:54), hu19A2.v12 (SEQ ID NO:55), hu19A2.v46 (SEQ ID NO:57), hu19A2.v52 (SEQ ID NO:57), hu19A2.v58 (SEQ ID NO:57), hu19A2.v60 (SEQ ID NO:57), hu19A2.v61 (SEQ ID NO:55), hu19A2.v62 (SEQ ID NO:55), hu19A2.v63 (SEQ ID NO:55), hu19A2.v64 (SEQ ID NO:60), hu19A2.v65 (SEQ ID NO:60), hu19A2.v66 (SEQ ID NO:55), hu19A2.v67 (SEQ ID NO:55), hu19A2.v68 (SEQ ID NO:60), hu19A2.v69 (SEQ ID NO:60), hu19A2.v70 (SEQ ID NO:54), hu19A2.v71 (SEQ ID NO:54), hu19A2.v72 (SEQ ID NO:54)).

FIG. 12 shows the amino acid sequence alignment of light chain and heavy chain variable regions of murine anti-CRTh2 antibody 8B1 and 3C12 and humanized anti-CRTh2 hu8B1.v1 (mu8B1—Light chain variable region (SEQ ID NO:50), mu8B1—Heavy chain variable region (SEQ ID NO:62); mu3C12—Light chain variable region (SEQ ID NO:51), mu3C12—Heavy chain variable region (SEQ ID NO:63); hu8B1.v1—Light chain variable region (SEQ ID NO:52), hu8B1.v1—Heavy chain variable region (SEQ ID NO:64)). Light chain and heavy chain Kabat CDR, Chothia CDR, and Contact CDR sequences of each antibody are provided.

FIG. 13 shows the amino acid sequence of murine anti-CRTh2 antibody 31A5. Light chain and heavy chain Kabat CDR, Chothia CDR, and Contact CDR sequences of antibody 31A5 are provided (mu31A5—Light chain variable sequence (SEQ ID NO:53), mu31A5—Heavy chain variable sequence (SEQ ID NO:65)).

FIG. 14 shows the amino acid sequence alignment of light chain and heavy chain variable regions of humanized anti-CRTh2 antibodies hu19A2.v1 and hu19A2.v52. FIG. 14A shows the light chain variable region sequence alignment (hu19A2.v1—Light chain variable region (SEQ ID NO:38); hu19A2.v52—Light chain variable region (SEQ ID NO:40)). Light chain Kabat CDR, Chothia CDR, and Contact CDR sequences of each antibody are provided. FIG. 14B shows the heavy chain variable region sequence alignment (hu19A2.v1—Heavy Chain variable region (SEQ ID NO:54); hu19A2.v52—Heavy Chain variable region (SEQ ID NO:57)). Heavy chain Kabat CDR, Chothia CDR, and Contact CDR sequences of each antibody are provided.

FIG. 15A-C show reactivity of humanized and humanized affinity matured anti-CRTh2 antibodies by flow cytometry with CRTh2 expressed on cell lines or with primary basophils and eosinophils. FIG. 15A shows reactivity by flow cytometry of 19A2 humanized (h19A2.v1) and humanized affinity matured (h19A2.v46, h19A2.v52) anti-CRTh2 antibodies at 1 ug/ml (black line) and 0.1 ug/ml (grey line) compared to control Ab (at 1 ug/ml, tinted histogram) with human CRTh2 expressed on 293 cells, as well as with wild-type 293 cells that do not express CRTh2. FIG. 15B shows reactivity by flow cytometry of humanized and humanized affinity matured 19A2 anti-CRTh2 antibodies (h19A2.v1, h19A2.v12, h19A2.v46, h19A2.v52) compared to control Ab (at 0.55 ug/ml, tinted histogram) with human, cynomolgus monkey or rhesus monkey CRTh2 expressed on 293 cells, as well as with wild-type 293 cells that do not express CRTh2. Primary antibody concentrations used were 0.55 ug/ml (black line), 0.18 ug/ml (very dark grey line), 0.06 ug/ml (dark grey line), 0.02 ug/ml (grey line) and 0.006 ug/ml (light grey line). FIG. 15C shows reactivity by flow cytometry of humanized affinity matured anti-human CRTh2 antibody h19A2.v52 to basophils and eosinophils on human, cynomolgus monkey or rhesus monkey PBMCs. PBMC were incubated with fluorescent-labeled anti-CRTh2 antibodies at 15 ug/ml (black line), 5 ug/ml (very dark grey line), 1.7 ug/ml (dark grey line), 0.6 ug/ml (grey line) or 0.2 ug/ml (light grey line) or with isotype control Ab at 15 ug/ml (tinted histogram) in combination with lineage-specific antibodies to detect basophils and eosinophils as described in material and methods.

FIG. 16A-D show Scatchard analysis of the binding affinities of anti-CRTh2 antibodies (Fab fragments) to surface expressed CRTh2 on 293 cells. FIG. 16A-B show the homologous competition radioligand cell binding assay of humanized h19A2.v52 Fab fragments to human or cynomolgus CRTh2 expressed on 293 cells. FIG. 16C-D show the homologous competition radioligand cell binding assay of humanized h19A2.v46 Fab fragments to human or cynomolgus CRTh2 expressed on 293 cells. The dissociation constant (K_{D}) for anti-CRTh2 Abs is indicated in the graphs. Bound/Total indicates the ratio of bound ¹²⁵I-labeled antibody and total ¹²⁵I-labeled antibody used in each assay.

FIG. 17A-B show the effect of anti-CRTh2 antibodies on PGD2-mediated inhibition of forskolin-induced cAMP levels or on forskolin-induced cAMP levels in 293 cells expressing human CRTh2. FIG. 17A shows that anti-CRTh2 antibody h19A2.v52 does not affect PGD2-mediated inhibition of forskolin-induced cAMP levels in 293 cells expressing human CRTh2. In comparison humanized h8B1 antibody blocked PGD2-mediated inhibition of forskolin-induced cAMP levels in a dose-dependent manner FIG. 17B shows that anti-CRTh2 antibodies h8B1 and h19A2.v52 do not affect forskolin-induced cAMP levels in the absence of PGD2 in 293 cells expressing human CRTh2. In comparison the ligand PGD2 reduced forskolin-induced cAMP levels in a dose-dependent manner.

FIG. 18A-C show that murine anti-CRTh2 antibody 19A2(mIgG2a) depletes basophils and eosinophils in vivo in blood, spleen and bone marrow in human CRTh2.Bac.Tg mice. Baseline numbers of CRTh2+ basophils (CD123+FceRI+) and eosinophils (CCR3+) was determined by flow cytometry from blood on day-7 (FIG. 18A and FIG. 18B) before treatment with the anti-CRTh2 Ab 19A2 as indicated. Human CRTh2.Bac.Tg mice were treated with anti-CRTh2 or isotype control antibodies at 20 ug/mouse or 100 ug/mouse i.v. Basophil and eosinophil depletion was assessed by flow cytometry on day 3 and day 7 as indicated in blood (FIG. 18A and FIG. 18B) or on day 7 in spleen and bone marrow (BM) (FIG. 18C). Symbols represent data from individual mice.

FIG. 19A-C show dose response and duration of basophil or eosinophil depletion after a single dose of humanized anti-CRTh2 antibody h19A2.v52 (hIgG1) in blood, spleen and bone marrow in human CRTh2.Bac.Tg mice. Baseline numbers of CRTh2+ eosinophils (CCR3+) was determined by flow cytometry from blood on day-3 (FIG. 19A) before treatment with h19A2.v52 as indicated. Human CRTh2.Bac.Tg mice were treated with anti-CRTh2 or isotype control antibodies at 10 ug/mouse or 200 ug/mouse i.v. Basophil and eosinophil depletion was assessed by flow cytometry on day 2, day 7 and day 14 as indicated in blood (FIG. 19A), spleen (FIG. 19B) and bone marrow (BM) (FIG. 19C). Symbols represent data from individual mice.

FIGS. 20A-B show that a depleting anti-CRTh2 19A2 mIgG2a antibody with effector function is more efficient than a non-depleting anti-CRTh2 19A2 mIgG2a_DANA Fc mutant antibody in innate immune cell depletion and reduction of Th2 BAL cytokine production in a TNP-OVA induced chronic asthma model in hCRTh2.Bac.Tg mice. FIG. 20A shows basophil, eosinophil and mast cell numbers that were assessed in lung tissue by flow cytometry and in BAL by differential cell count combined with flow cytometry (FIG. 20A). Percent depletion by anti-CRTh2 19A2 mIgG2a antibodies and the Fc mutant 19A2 mIgG2a_DANA antibodies as compared to anti-ragweed isotype control antibodies is indicated in the graphs. FIG. 20B shows the concentrations of IL-4 determined by ELISA in BAL. Percent reduction by anti-CRTh2 19A2 mIgG2a antibodies and the Fc mutant 19A2 mIgG2a_DANA antibodies as compared to anti-ragweed isotype control antibodies is indicated in the graphs.

### DETAILED DESCRIPTION OF EMBODIMENTS OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “CRTh2” as used herein, refers to any native CRTh2 from any mammals such as primates (e.g., human, rhesus, cynomologous CRTh2) and rodents (e.g., mice and rats), unless otherwise indicated. The term encompasses “full-length,” unprocessed CRTh2 as well as any form of CRTh2 that results from processing in the cell. The term also encompasses naturally occurring variants of CRTh2, e.g., splice variants or allelic variants. The amino acid sequence of an exemplary human CRTh2 is shown in SEQ ID NO:84. The amino acid sequence of an exemplary rhesus CRTh2 is shown in SEQ ID NO:85. The amino acid sequence of an exemplary cynomologous CRTh2 is shown in SEQ ID NO:86. See e.g., L. Cosmi et al., Eur. J. Immunol. 30(10):2972-9 (2000); K. Nagat et al., FEBS Lett. 459(2): 195-9 (1999); and K. Nagata et al., J. Immunol. 162(3): 1278-86 (1999).

HUMAN CRTH2 SEQUENCE
(SEQ ID NO: 84)
MSANATLKPLCPILEQMSRLQSHSNTSIRYIDHAAVLLHGLASLLGLVEN
GVILFVVGCRMRQTVVTTWVLHLALSDLLASASLPFFTYFLAVGHSWELG
TTFCKLHSSIFFLNMFASGFLLSAISLDRCLQVVRPVWAQNHRTVAAAHK
VCLVLWALAVLNTVPYFVFRDTISRLDGRIMCYYNVLLLNPGPDRDATCN
SRQAALAVSKFLLAFLVPLAIIASSHAAVSLRLQHRGRRRPGRFVRLVAA
VVAAFALCWGPYHVFSLLEARAHANPGLRPLVWRGLPFVTSLAFFNSVAN
PVLYVLTCPDMLRKLRRSLRTVLESVLVDDSELGGAGSSRRRRTSSTARS
ASPLALCSRPEEPRGPARLLGWLLGSCAASPQTGPLNRALSSTSS
RHESUS CRTH2 SEQUENCE (NCBI REFERENCE NUMBER
XM_001084746)
(SEQ ID NO: 85)
MSANATLKPLCPILEEMSHLRSHSNTSIRYIDHATVLLHGLASLLGLVEN
GVILFVVGCRMRQTVVTTWVLHLALSDLLASASLPFFTYFLAVGHSWELG
TTFCKLHSSIFFLNMFASGFLLSAISLDRCLQVVWPVWAQNHRTVAAAHK
VCLVLWALAVLNTVPYFVFRDTISRLDGRIMCYYNVLLLNPGPDRDATCN
SRQAALAVSKFLLAFLVPLAIIASSHAAVSLRLQHRGRRRPGRFVRLVAA
VVAAFALCWGPYHVFSLLEARAHANPGLRPLVWRGLPFVTSLAFFNSVAN
PVLYVLTCPDMLRKLRRSLRTVLESVLVDDSELGGAGSSRRRRRTPSTAR
SASSLALSSRPEERRGPARLFGWLLGGCAASPQRGPLNRALSSTSS
CYNO CRTH2 SEQUENCE
(SEQ ID NO: 86)
MSANATLKPLCPILEEMSHLRSHSNTSIRYIDHATVLLHGLASLLGLVEN
GVILFVVGCRMRQTVVTTWVLHLALSDLLASASLPFFTYFLAVGHSWELG
TTFCKLHSSIFFLNMFASGFLLSAISLDRCLQVVWPVWAQNHRTVAAAHK
VCLVLWALAVLNTVPYFVFRDTISRLDGRIMCYYNVLLLNPGSDRDATCN
SRQAALAVSKFLLAFLVPLAIIASSHAAVSLRLQHRGRRRPGRFVRLVAA
VVAAFALCWGPYHVFSLLEARAHANRGLRPLVWRGLPFVTSLAFFNSVAN
PVLYVLTCPDMLRKLRRSLRTVLESVLVDDSELGGAGSSRRRRRTPSTAR
SASSLALSSHPEERRGPARLFGWLLGGCAASPQRGPLNRALSSTSS

The terms “anti-CRTh2 antibody” and “an antibody that binds to CRTh2” refer to an antibody that is capable of binding CRTh2 with sufficient affinity such that the antibody is useful as a diagnostic and/or therapeutic agent in targeting CRTh2. In one embodiment, the extent of binding of an anti-CRTh2 antibody to an unrelated, non-CRTh2 protein is less than about 10% of the binding of the antibody to CRTh2 as measured, e.g., by a radioimmunoassay (RIA). In certain embodiments, an antibody that binds to CRTh2 has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M). In certain embodiments, an anti-CRTh2 antibody binds to an epitope of CRTh2 that is conserved among CRTh2 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, 1-R2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. An HVR region as used herein comprise any number of residues located within positions 24-36 (for L1), 46-56 (for L2), 89-97 (for L3), 26-35B (for H1), 47-65 (for H2), and 93-102 (for H3). Therefore, an HVR includes residues in positions described previously:

A) 24-34 (L1), 50-52 (L2), 91-96 (L3), 26-32 (H1), 53-55 (H2), and 96-101 (H3) (Chothia and Lesk, J. Mol. Biol. 196:901-917 (1987);

B) 24-34 of L1, 50-56 of L2, 89-97 of L3, 31-35B of H1, 50-65 of H2, and 95-102 of H3 (Kabat et al., Sequences of Proteins of Immunological Interest, 5th Ed. Public Health Service, National Institutes of Health, Bethesda, Md. (1991).

C) 30-36 (L1), 46-55 (L2), 89-96 (L3), 30-35 (H1), 47-58 (H2), 93-100a-j (H3) (MacCallum et al. J. Mol. Biol. 262:732-745 (1996).

With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-CRTh2 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. In some embodiments, the treatment improves asthma control, reduces asthma exacerbations, improves lung function, and/or improves patient reported symptoms. An individual is successfully “treated”, for example, if one or more symptoms associated with the disorder are mitigated or eliminated.

As used herein, “in conjunction with” refers to administration of one treatment modality in addition to another treatment modality. As such, “in conjunction with” refers to administration of one treatment modality before, during or after administration of the other treatment modality to the individual.

As used herein, the term “prevention” includes providing prophylaxis with respect to occurrence or recurrence of a disease in an individual. An individual may be predisposed to a disorder, susceptible to a disorder, or at risk of developing a disorder, but has not yet been diagnosed with the disorder. In some embodiments, anti-CRTh2 antibodies described herein are used to delay development of the disorder. In some embodiments, the anti-CRTh2 antibodies described herein prevents asthma exacerbations and/or decline in lung function or asthma states.

As used herein, an individual “at risk” of developing a disorder may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of the disorder, as known in the art. An individual having one or more of these risk factors has a higher probability of developing the disorder than an individual without one or more of these risk factors.

An “effective amount” refers to at least an amount effective, at dosages and for periods of time necessary, to achieve the desired or indicated effect, including a therapeutic or prophylactic result. An effective amount can be provided in one or more administrations.

A “therapeutically effective amount” is at least the minimum concentration required to effect a measurable improvement of a particular disorder. A therapeutically effective amount herein may vary according to factors such as the disease state, age, sex, and weight of the patient, and the ability of the antibody to elicit a desired response in the individual. A therapeutically effective amount is also one in which any toxic or detrimental effects of the antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in subjects prior to or at the earlier stage of disease, the prophylactically effective amount can be less than the therapeutically effective amount.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ADCC, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed.

The term “asthma” refers to a complex disorder characterized by variable and recurring symptoms, reversible airflow obstruction (e.g., by bronchodilator) and bronchial hyperresponsiveness which may or may not be associated with underlying inflammation. Examples of asthma include aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids and other asthmas as mentioned in J Allergy Clin Immunol (2010) 126(5):926-938. Symptoms of asthma include shortness of breath, cough (changes in sputum production and/or sputum quality and/or cough frequency), wheezing, chest tightness, bronchioconstriction and nocturnal awakenings ascribed to one of the symptoms above or a combination of these symptoms (Juniper et al (2000) Am. J. Respir. Crit. Care Med., 162(4), 1330-1334.).

The term “mild asthma” refers to a patient generally experiencing symptoms or exacerbations less than two times a week, nocturnal symptoms less than two times a month, and is asymptomatic between exacerbations. Mild, intermittent asthma is often treated as needed with the following: inhaled bronchodilators (short-acting inhaled beta2-agonists); avoidance of known triggers; annual influenza vaccination; pneumococcal vaccination every 6 to 10 years, and in some cases, an inhaled beta2-agonist, cromolyn, or nedocromil prior to exposure to identified triggers. If the patient has an increasing need for short-acting beta2-agonist (e.g., uses short-acting beta2-agonist more than three to four times in 1 day for an acute exacerbation or uses more than one canister a month for symptoms), the patient may require a stepup in therapy.

The term “moderate asthma” generally refers to asthma in which the patient experiences exacerbations more than two times a week and the exacerbations affect sleep and activity; the patient has nighttime awakenings due to asthma more than two times a month; the patient has chronic asthma symptoms that require short-acting inhaled beta2-agonist daily or every other day; and the patient's pretreatment baseline peak expiratory flow (PEF) or forced expiratory volume in 1 second (FEV1) is 60 to 80 percent predicted and PEF variability is 20 to 30 percent.

The term “severe asthma” generally refers to asthma in which the patient has almost continuous symptoms, frequent exacerbations, frequent nighttime awakenings due to the asthma, limited activities, PEF or FEV1 baseline less than 60 percent predicted, and PEF variability of 20 to 30 percent.

The term “FEV1” refers to the volume of air exhaled in the first second of a forced expiration. It is a measure of airway obstruction. FEV1 may be noted in other similar ways, e.g., FEVₛ, and it should be understood that all such similar variations have the same meaning.

The term “corticosteroid” includes glucocorticoids and mineralocorticoids. For example, corticosteroid includes, but is not limited to fluticasone (including fluticasone propionate (FP)), beclometasone, budesonide, ciclesonide, mometasone, flunisolide, betamethasone, hydrocortisone, prednisone, prednisolone, methylprednisolone, and triamcinolone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, triamcinolone acetonide and any other corticosteroid currently available or becoming available in the future. Examples of corticosteroids that can be inhaled and are combined with a long-acting beta2-agonist include, but are not limited to: budesonide/formoterol and fluticasone/salmeterol.

The term “cytokine” is a generic term for proteins released by one cell population that act on another cell as intercellular mediators. Examples of such cytokines are lymphokines, monokines; interleukins (ILs) such as IL-1, IL-1α, IL-2, IL-3, IL-4, IL-5, IL-6, IL-7, IL-8, IL-9, IL-11, IL-12, IL-13, IL-15, including PROLEUKIN® rIL-2; a tumor-necrosis factor such as TNF-α or TNF-β; and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the invention described herein include “comprising,” “consisting,” and “consisting essentially of” aspects and embodiments.

### II. Compositions and Methods

In one aspect, provided herein are antibodies that bind CRTh2. In certain embodiments, the anti-CRTh2 binds to human CRTh2 and depletes CRTh2 expressing cells when an effective amount is administered to a subject (e.g., a human subject). In some embodiments, the anti-CRTh2 antibody also binds to CRTh2 of a non-human primate (e.g., rhesus or cynomologous CRTh2). Antibodies of the invention are useful, e.g., for the diagnosis or treatment of a disorder mediated by CRTh2 expressing cells.

### Exemplary Anti-CRTh2 Antibodies

In one aspect, the invention provides isolated antibodies that bind to CRTh2. In certain embodiments, an anti-CRTh2 antibody has one or more of the following characteristics: (1) binds CRTh2 (e.g., human CRTh2) and depletes CRTh2 expressing cells (e.g., Th2 cells, mast cells, eosinophils, basophils, and/or innate type 2 (IT2) cells) when an effective amount is administered to a subject; (2) has been engineered to improve ADCC; (3) is afucosylated or has reduced fucosylation; (4) competitively inhibits binding of at least one of the following antibodies: 19A2, 8B1, 31A5, 3C12, and any of the humanized antibodies described herein to human CRTh2; (5) binds to an epitope of human CRTh2 that is the same as or overlaps with the CRTh2 epitope bound by at least one of the following anti-CRTh2 antibodies: 19A2, 8B1, 31A5, 3C12, and any of the humanized antibodies described herein; (6) binds to CRTh2 of a human and a non-human primate (e.g., rhesus and/or cynomologous CRTh2); (7) blocks CRTh2 signaling; (8) prevents recruitment of CRTh2 expressing cells in response to prostaglandin D2; (9) blocks Ca2⁺ flux in CRTh2 expressing cells; (10) does not exhibit agonistic activity; (11) does not reduce forskolin-induced cAMP level in CRTh2 expressing cells (e.g., 293 cells expressing human CRTh2); and (12) blocks prostaglandin D2 triggered inhibition of forskolin-induced cAMP levels in CRTh2 expressing cells (e.g., 293 cells expressing human CRTh2).

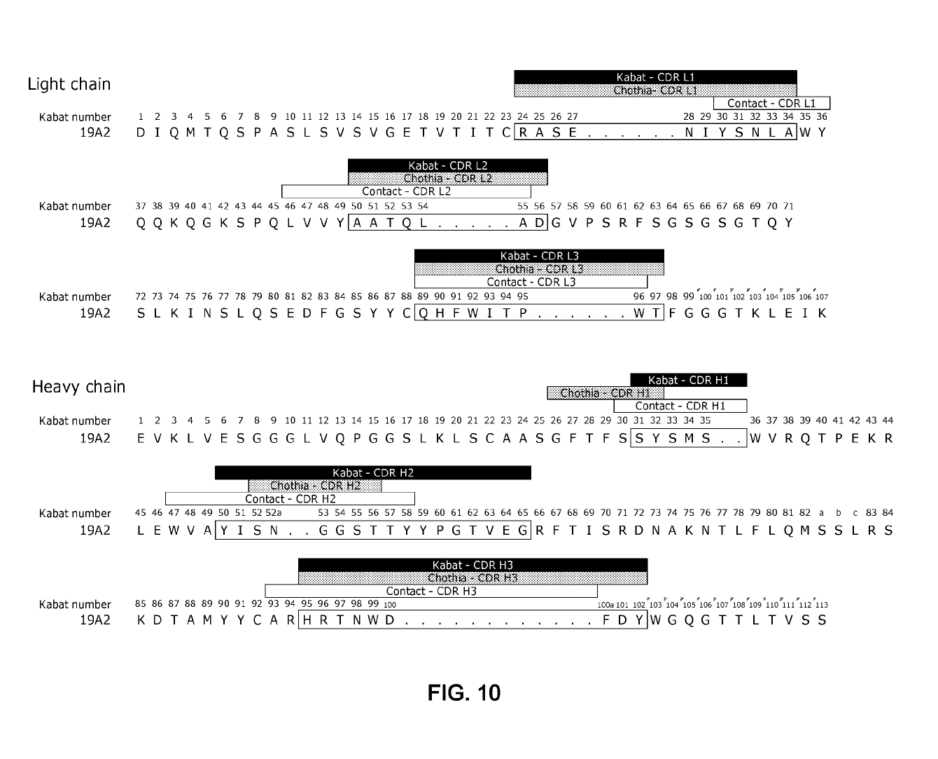

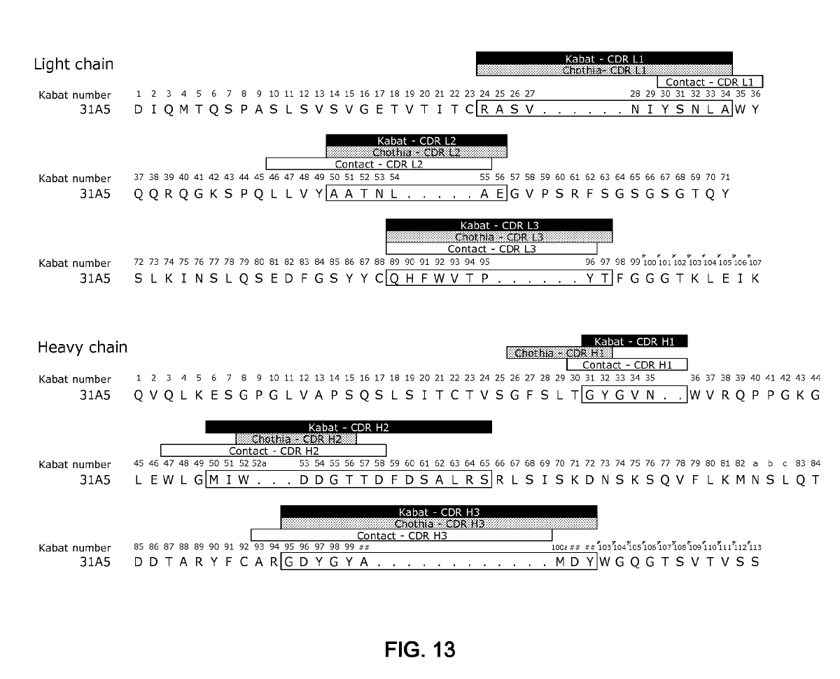

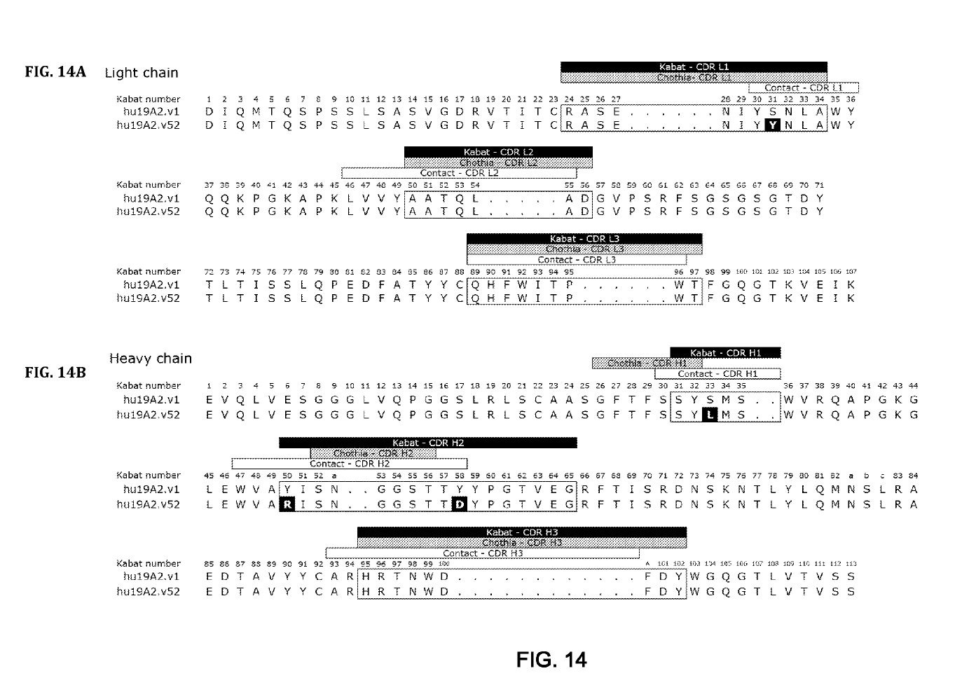

In another aspect, the invention provides an isolated anti-CRTh2 antibody comprising (a) a light chain variable region comprising at least one, two, or three HVRs selected from HVR-L1, HVR-L2, and HVR-L3 of any one of murine antibody 19A2, 8B1, 31A5, and 3C12, and humanized antibodies described herein (e.g., hu8B1.v1, hu19A2.v1, v12, v38, v46, v47, v51-v53, v57, v58, and v60-v72); and/or (b) a heavy chain variable region comprising at least one, two, or three HVRs selected from HVR-H1, HVR-H2, and HVR-H3 of any one of murine antibody 19A2, 8B1, 3C12, and 31A5, and humanized antibodies described herein (e.g., hu8B1.v1, hu19A2.v1, v12, v38, v46, v47, v51-v53, v57, v58, and v60-v72). In some embodiments, the HVR-L1, HVR-L2, HVR-L3, HVR-H1, HVR-H2, and HVR-H3 comprise Kabat CDR, Chothia CDR, or Contact CDR sequences as shown in FIGS. 10, 11A, 11B, 12, 13, and 14.

In another aspect, the invention provides an anti-CRTh2 antibody comprising at least one, two, three, four, five, or six HVRs selected from (i) HVR-L1 comprising the amino acid sequence of SEQ ID NO:22 or 23; (ii) HVR-L2 comprising the amino acid sequence of SEQ ID NO:25; (iii) HVR-L3 comprising the amino acid sequence of SEQ ID NO:27; (iv) HVR-H1 comprising the amino acid sequence of SEQ ID NO:29 or 30; (v) HVR-H2 comprising the amino acid sequence of SEQ ID NO:32 or 33; (vi) HVR-H3 comprising the amino acid sequence of SEQ ID NO:35 or 36.

In another aspect, the invention provides an anti-CRTh2 antibody comprising at least one, two, three, four, five, or six HVRs selected from (i) HVR-L1 comprising the amino acid sequence of SEQ ID NO:24; (ii) HVR-L2 comprising the amino acid sequence of SEQ ID NO:26; (iii) HVR-L3 comprising the amino acid sequence of SEQ ID NO:28; (iv) HVR-H1 comprising the amino acid sequence of SEQ ID NO:31; (v) HVR-H2 comprising the amino acid sequence of SEQ ID NO:34; (vi) HVR-H3 comprising the amino acid sequence of SEQ ID NO:37.

In another aspect, the invention provides an anti-CRTh2 antibody comprising at least one, two, three, four, five, or six HVRs selected from (i) HVR-L1 comprising the amino acid sequence of RASENIYXNLA (SEQ ID NO:1), wherein X is S, W, or Y; (ii) HVR-L2 comprising the amino acid sequence of AATQLAX (SEQ ID NO:2), wherein X is D, E, or S; (iii) HVR-L3 comprising the amino acid sequence of QHFWITPWT (SEQ ID NO:3); (iv) HVR-H1 comprising the amino acid sequence of X₁YX₂MS (SEQ ID NO:4), wherein X₁ is S or F, and X₂ is S, L, or K; (v) HVR-H2 comprising the amino acid sequence of X₁ISNGGSTTX₂YPGTVEG (SEQ ID NO:5), wherein X₁ is Y or R, and X₂ is Y or D; (vi) HVR-H3 comprising the amino acid sequence of HRTNWDFDY (SEQ ID NO:6).

In one aspect, the invention provides an antibody comprising at least one, at least two, or all three VH HVR sequences selected from (a) HVR-H1 comprising the amino acid sequence of SEQ ID NO:29 or 30; (b) HVR-H2 comprising the amino acid sequence of SEQ ID NO:32 or 33; and (c) HVR-H3 comprising the amino acid sequence of SEQ ID NO:35 or 36. In one embodiment, the antibody comprises HVR-H3 comprising the amino acid sequence of SEQ ID NO:35 or 36. In another embodiment, the antibody comprises HVR-H3 comprising the amino acid sequence of SEQ ID NO:35 or 36 and HVR-L3 comprising the amino acid sequence of SEQ ID NO:27. In a further embodiment, the antibody comprises HVR-H3 comprising the amino acid sequence of SEQ ID NO:35 or 36, HVR-L3 comprising the amino acid sequence of SEQ ID NO:27, and HVR-H2 comprising the amino acid sequence of SEQ ID NO:32 or 33. In a further embodiment, the antibody comprises (a) HVR-H1 comprising the amino acid sequence of SEQ ID NO:29 or 30; (b) HVR-H2 comprising the amino acid sequence of SEQ ID NO:32 or 33; and (c) HVR-H3 comprising the amino acid sequence of SEQ ID NO:35 or 36.

In another aspect, the invention provides an antibody comprising at least one, at least two, or all three VL HVR sequences selected from (a) HVR-L1 comprising the amino acid sequence of SEQ ID NO:22 or 23; (b) HVR-L2 comprising the amino acid sequence of SEQ ID NO:25; and (c) HVR-L3 comprising the amino acid sequence of SEQ ID NO:27. In one embodiment, the antibody comprises (a) HVR-L1 comprising the amino acid sequence of SEQ ID NO:22 or 23; (b) HVR-L2 comprising the amino acid sequence of SEQ ID NO:25; and (c) HVR-L3 comprising the amino acid sequence of SEQ ID NO:27.

In one aspect, the invention provides an antibody comprising at least one, at least two, or all three VH HVR sequences selected from (a) HVR-H1 comprising the amino acid sequence of SEQ ID NO:31; (b) HVR-H2 comprising the amino acid sequence of SEQ ID NO:34; and (c) HVR-H3 comprising the amino acid sequence of SEQ ID NO:37. In one embodiment, the antibody comprises HVR-H3 comprising the amino acid sequence of SEQ ID NO:37. In another embodiment, the antibody comprises HVR-H3 comprising the amino acid sequence of SEQ ID NO:37 and HVR-L3 comprising the amino acid sequence of SEQ ID NO:28. In a further embodiment, the antibody comprises HVR-H3 comprising the amino acid sequence of SEQ ID NO:37, HVR-L3 comprising the amino acid sequence of SEQ ID NO:28, and HVR-H2 comprising the amino acid sequence of SEQ ID NO:34. In a further embodiment, the antibody comprises (a) HVR-H1 comprising the amino acid sequence of SEQ ID NO:31; (b) HVR-H2 comprising the amino acid sequence of SEQ ID NO:34; and (c) HVR-H3 comprising the amino acid sequence of SEQ ID NO:37.

In another aspect, the invention provides an antibody comprising at least one, at least two, or all three VL HVR sequences selected from (a) HVR-L1 comprising the amino acid sequence of SEQ ID NO:24; (b) HVR-L2 comprising the amino acid sequence of SEQ ID NO:26; and (c) HVR-L3 comprising the amino acid sequence of SEQ ID NO:28. In one embodiment, the antibody comprises (a) HVR-L1 comprising the amino acid sequence of SEQ ID NO:24; (b) HVR-L2 comprising the amino acid sequence of SEQ ID NO:26; and (c) HVR-L3 comprising the amino acid sequence of SEQ ID NO:28.

In one aspect, the invention provides an antibody comprising at least one, at least two, or all three VH HVR sequences selected from (a) HVR-H1 comprising the amino acid sequence of SEQ ID NO:13, 14, 15, 16, or 17; (b) HVR-H2 comprising the amino acid sequence of SEQ ID NO:18, 19, 20, or 21; and (c) HVR-H3 comprising the amino acid sequence of SEQ ID NO: 6. In one embodiment, the antibody comprises HVR-H3 comprising the amino acid sequence of SEQ ID NO:6. In another embodiment, the antibody comprises HVR-H3 comprising the amino acid sequence of SEQ ID NO:6 and HVR-L3 comprising the amino acid sequence of SEQ ID NO:3. In a further embodiment, the antibody comprises HVR-H3 comprising the amino acid sequence of SEQ ID NO: 6, HVR-L3 comprising the amino acid sequence of SEQ ID NO:3, and HVR-H2 comprising the amino acid sequence of SEQ ID NO:18, 19, 20, or 21. In a further embodiment, the antibody comprises (a) HVR-H1 comprising the amino acid sequence of SEQ ID NO:13, 14, 15, 16, or 17; (b) HVR-H2 comprising the amino acid sequence of SEQ ID NO:18, 19, 20, or 21; and (c) HVR-H3 comprising the amino acid sequence of SEQ ID NO: 6.

In another aspect, the invention provides an antibody comprising at least one, at least two, or all three VL HVR sequences selected from (a) HVR-L1 comprising the amino acid sequence of SEQ ID NO:7, 8, or 9; (b) HVR-L2 comprising the amino acid sequence of SEQ ID NO:10, 11, or 12; and (c) HVR-L3 comprising the amino acid sequence of SEQ ID NO:3. In one embodiment, the antibody comprises (a) HVR-L1 comprising the amino acid sequence of SEQ ID NO: 7, 8, or 9; (b) HVR-L2 comprising the amino acid sequence of SEQ ID NO: 10, 11, or 12; and (c) HVR-L3 comprising the amino acid sequence of SEQ ID NO:3.

In another aspect, an antibody of the invention comprises (a) a VH domain comprising at least one, at least two, or all three VH HVR sequences selected from (i) HVR-H1 comprising the amino acid sequence of SEQ ID NO:29 or 30, (ii) HVR-H2 comprising the amino acid sequence of SEQ ID NO:32 or 33, and (iii) HVR-H3 comprising the amino acid sequence selected from SEQ ID NO:35 or 36; and (b) a VL domain comprising at least one, at least two, or all three VL HVR sequences selected from (i) HVR-L1 comprising the amino acid sequence of SEQ ID NO:22 or 23, (ii) HVR-L2 comprising the amino acid sequence of SEQ ID NO:25, and (c) HVR-L3 comprising the amino acid sequence of SEQ ID NO:27.

In another aspect, the invention provides an antibody comprising (a) HVR-H1 comprising the amino acid sequence of SEQ ID NO:29 or 30; (b) HVR-H2 comprising the amino acid sequence of SEQ ID NO:32 or 33; (c) HVR-H3 comprising the amino acid sequence of SEQ ID NO:35 or 36; (d) HVR-L1 comprising the amino acid sequence of SEQ ID NO:22 or 23; (e) HVR-L2 comprising the amino acid sequence of SEQ ID NO:25; and (f) HVR-L3 comprising the amino acid sequence of SEQ ID NO:26.

In another aspect, an antibody of the invention comprises (a) a VH domain comprising at least one, at least two, or all three VH HVR sequences selected from (i) HVR-H1 comprising the amino acid sequence of SEQ ID NO:31, (ii) HVR-H2 comprising the amino acid sequence of SEQ ID NO:34, and (iii) HVR-H3 comprising the amino acid sequence selected from SEQ ID NO:37; and (b) a VL domain comprising at least one, at least two, or all three VL HVR sequences selected from (i) HVR-L1 comprising the amino acid sequence of SEQ ID NO:24, (ii) HVR-L2 comprising the amino acid sequence of SEQ ID NO:26, and (c) HVR-L3 comprising the amino acid sequence of SEQ ID NO:28.

In another aspect, the invention provides an antibody comprising (a) HVR-H1 comprising the amino acid sequence of SEQ ID NO:31; (b) HVR-H2 comprising the amino acid sequence of SEQ ID NO:34; (c) HVR-H3 comprising the amino acid sequence of SEQ ID NO:37; (d) HVR-L1 comprising the amino acid sequence of SEQ ID NO:24; (e) HVR-L2 comprising the amino acid sequence of SEQ ID NO:26; and (f) HVR-L3 comprising the amino acid sequence of SEQ ID NO:28.

In another aspect, an antibody of the invention comprises (a) a VH domain comprising at least one, at least two, or all three VH HVR sequences selected from (i) HVR-H1 comprising the amino acid sequence of SEQ ID NO:13, 14, 15, 16, or 17, (ii) HVR-H2 comprising the amino acid sequence of SEQ ID NO:18, 19, 20, or 21, and (iii) HVR-H3 comprising the amino acid sequence of SEQ ID NO: 6; and (b) a VL domain comprising at least one, at least two, or all three VL HVR sequences selected from (i) HVR-L1 comprising the amino acid sequence of SEQ ID NO:7, 8, or 9, (ii) HVR-L2 comprising the amino acid sequence of SEQ ID NO:10, 11, or 12, and (c) HVR-L3 comprising the amino acid sequence of SEQ ID NO:3.

In another aspect, the invention provides an antibody comprising (a) HVR-H1 comprising the amino acid sequence of SEQ ID NO:13, 14, 15, 16, or 17; (b) HVR-H2 comprising the amino acid sequence of SEQ ID NO:18, 19, 20, or 21; (c) HVR-H3 comprising the amino acid sequence of SEQ ID NO:6; (d) HVR-L1 comprising the amino acid sequence of SEQ ID NO:7, 8, or 9; (e) HVR-L2 comprising the amino acid sequence of SEQ ID NO:10, 11, 12; and (f) HVR-L3 comprising the amino acid sequence of SEQ ID NO:3. In some embodiments, the antibody comprises (a) HVR-H1 comprising the amino acid sequence of SEQ ID NO:13; (b) HVR-H2 comprising the amino acid sequence of SEQ ID NO:18; (c) HVR-H3 comprising the amino acid sequence of SEQ ID NO:6; (d) HVR-L1 comprising the amino acid sequence of SEQ ID NO:7; (e) HVR-L2 comprising the amino acid sequence of SEQ ID NO:10; and (f) HVR-L3 comprising the amino acid sequence of SEQ ID NO:3. In some embodiments, the antibody comprises (a) HVR-H1 comprising the amino acid sequence of SEQ ID NO:13; (b) HVR-H2 comprising the amino acid sequence of SEQ ID NO:19; (c) HVR-H3 comprising the amino acid sequence of SEQ ID NO:6; (d) HVR-L1 comprising the amino acid sequence of SEQ ID NO:8; (e) HVR-L2 comprising the amino acid sequence of SEQ ID NO:10; and (f) HVR-L3 comprising the amino acid sequence of SEQ ID NO:3. In some embodiments, the antibody comprises (a) HVR-H1 comprising the amino acid sequence of SEQ ID NO:15; (b) HVR-H2 comprising the amino acid sequence of SEQ ID NO:20; (c) HVR-H3 comprising the amino acid sequence of SEQ ID NO:6; (d) HVR-L1 comprising the amino acid sequence of SEQ ID NO:9; (e) HVR-L2 comprising the amino acid sequence of SEQ ID NO:11; and (f) HVR-L3 comprising the amino acid sequence of SEQ ID NO:3. In some embodiments, the antibody comprises (a) HVR-H1 comprising the amino acid sequence of SEQ ID NO:15; (b) HVR-H2 comprising the amino acid sequence of SEQ ID NO:20; (c) HVR-H3 comprising the amino acid sequence of SEQ ID NO:6; (d) HVR-L1 comprising the amino acid sequence of SEQ ID NO:9; (e) HVR-L2 comprising the amino acid sequence of SEQ ID NO:10; and (f) HVR-L3 comprising the amino acid sequence of SEQ ID NO:3.

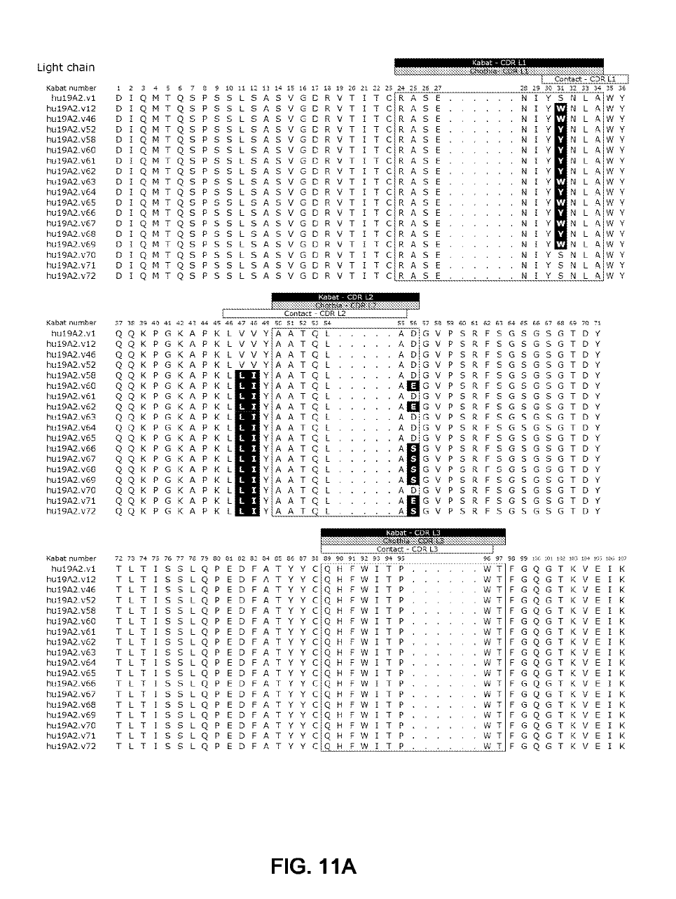

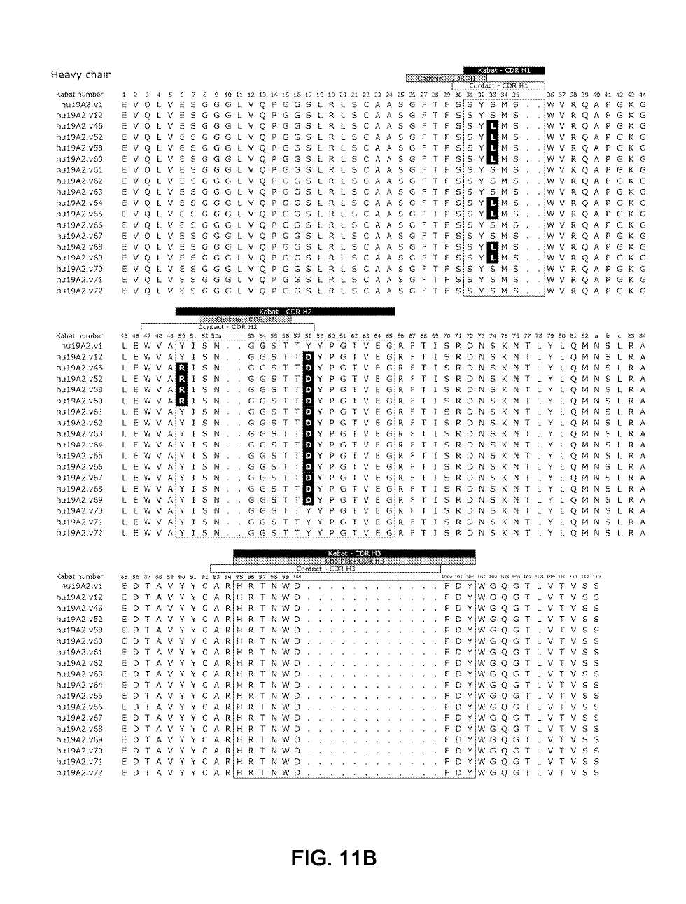

In any of the above embodiments, an anti-CRTh2 antibody is an isolated antibody. In any of the above embodiments, an anti-CRTh2 antibody is humanized. In one embodiment, an anti-CRTh2 antibody comprises HVRs as in any of the above embodiments and HVRs (including HVRs comprising Kabat CDR, Chothia CDR, or Contact CDR sequences) shown in FIGS. 10, 11, 12, 13, and 14, and further comprises an acceptor human framework, e.g. a human immunoglobulin framework or a human consensus framework. In another embodiment, an anti-CRTh2 antibody comprises HVRs as in any of the above embodiments, and further comprises a VL comprising an FR (e.g., FR1, 1-R2, FR3, or FR4) sequence as shown in FIG. 11A, 12, and 14A. In another embodiment, an anti-CRTh2 antibody comprises HVRs as in any of the above embodiments and HVRs (including HVRs comprising Kabat CDR, Chothia CDR, or Contact CDR sequences) shown in FIGS. 10, 11, 12, 13, and 14, and further comprises a VH comprising an FR (e.g., 1-R1, FR2, FR3, or FR) sequence as shown in FIGS. 11B, 12, and 14B.

In certain embodiments, an anti-CRTh2 antibody described herein comprises HVRs as defined by Kabat, e.g., an anti-CRTh2 antibody comprising CDR-H1, CDR-H2, CDR-H3, CDR-L1, CDR-L2, and CDR-L3, wherein each of the CDRs is defined by Kabat as further described herein. In certain embodiments, an anti-CRTh2 antibody described herein comprises HVRs as defined by Chothia, e.g., an anti-CRTh2 antibody comprising CDR-H1, CDR-H2, CDR-H3, CDR-L1, CDR-L2, and CDR-L3, wherein each of the CDRs is defined by Chothia as further described herein. In certain embodiments, an anti-CRTh2 antibody described herein comprises HVRs as defined by Contact CDR sequences, e.g., an anti-CRTh2 antibody comprising CDR-H1, CDR-H2, CDR-H3, CDR-L1, CDR-L2, and CDR-L3, wherein each of the CDRs is defined by Contact CDR sequences as further described herein.

In another aspect, an anti-CRTh2 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence selected from the group consisting of SEQ ID NOS:38-53. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CRTh2 antibody comprising that sequence retains the ability to bind to CRTh2. In certain embodiments, a total of 1, 2, 3, 4, 5, 6, 7, 8, 9, or 10 amino acids have been substituted, inserted and/or deleted in any of SEQ ID NOS:38-53. In certain embodiments, the substitutions, insertions, or deletions occur in regions outside the HVRs (i.e., in the FRs). Optionally, the anti-CRTh2 antibody comprises the VL sequence selected from the group consisting of SEQ ID NOS:38-53, including post-translational modifications of that sequence. In a particular embodiment, the VL comprises one, two or three HVRs selected from (a) HVR-L1 comprising the amino acid sequence selected from the group consisting of SEQ ID NOS:7-9; (b) HVR-L2 comprising the amino acid sequence selected from the group consisting of SEQ ID NOS:10-12; and (c) HVR-L3 comprising the amino acid sequence of SEQ ID NO:3. In a particular embodiment, the VL comprises one, two or three HVRs selected from (a) HVR-L1 comprising the amino acid sequence of SEQ ID NO:22 or 23; (b) HVR-L2 comprising the amino acid sequence of SEQ ID NO:25; and (c) HVR-L3 comprising the amino acid sequence of SEQ ID NO:27. In a particular embodiment, the VL comprises one, two or three HVRs selected from (a) HVR-L1 comprising the amino acid sequence of SEQ ID NO:24; (b) HVR-L2 comprising the amino acid sequence of SEQ ID NO:26; and (c) HVR-L3 comprising the amino acid sequence of SEQ ID NO:28.

In another aspect, an anti-CRTh2 antibody comprises a heavy chain variable domain (VH) sequence having at least 90%, 91%, 92%, 93%, 94%, 95%, 96%, 97%, 98%, 99%, or 100% sequence identity to the amino acid sequence selected from the group consisting of SEQ ID NOS:54-65. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-CRTh2 antibody comprising that sequence retains the ability to bind to CRTh2. In certain embodiments, a total of 1, 2, 3, 4, 5, 6, 7, 8, 9, or 10 amino acids have been substituted, inserted and/or deleted in any of SEQ ID NOS:54-65. In certain embodiments, substitutions, insertions, or deletions occur in regions outside the HVRs (i.e., in the FRs). Optionally, the anti-CRTh2 antibody comprises the VH sequence in any of SEQ ID NOS:54-65, including post-translational modifications of that sequence. In a particular embodiment, the VH comprises one, two or three HVRs selected from: (a) HVR-H1 comprising the amino acid sequence selected from the group consisting of SEQ ID NOS:13-17, (b) HVR-H2 comprising the amino acid sequence selected from the group consisting of SEQ ID NOS:18-21, and (c) HVR-H3 comprising the amino acid sequence of SEQ ID NO:6. In a particular embodiment, the VH comprises one, two or three HVRs selected from: (a) HVR-H1 comprising the amino acid sequence of SEQ ID NO:29 or 30, (b) HVR-H2 comprising the amino acid sequence of SEQ ID NO:32 or 33, and (c) HVR-H3 comprising the amino acid sequence of SEQ ID NO:35 or 36. In a particular embodiment, the VH comprises one, two or three HVRs selected from: (a) HVR-H1 comprising the amino acid sequence of SEQ ID NO:31, (b) HVR-H2 comprising the amino acid sequence of SEQ ID NO:34, and (c) HVR-H3 comprising the amino acid sequence of SEQ ID NO:37.

In another aspect, an anti-CRTh2 antibody is provided, wherein the antibody comprises a VH as in any of the embodiments provided above, and a VL as in any of the embodiments provided above. In some embodiments, the antibody comprises the VH sequence of any of murine antibody 8B1, 3C12, 31A5, and 19A2, and humanized antibody hu19A2 (including, v1, v12, v38, v46, v47, v51-v53, v57, v58, and v60-v72). In some embodiments, the antibody comprises the VL sequence of any of murine antibody 8B1, 3C12, 31A5, and 19A2, and humanized antibody hu19A2 (including, v1, v12, v38, v46, v47, v51-v53, v57, v58, and v60-v72). In one embodiment, the antibody comprises a VH sequence selected from the group consisting of SEQ ID NO:54-60 and a VL sequence selected from the group consisting of SEQ ID NO:38-48, including post-translational modifications of those sequences. In one embodiment, the antibody comprises the VH sequence of SEQ ID NO:55 and the VL sequence of SEQ ID NO:39, including post-translational modifications of those sequences. In one embodiment, the antibody comprises the VH sequence of SEQ ID NO:57 and the VL sequence of SEQ ID NO:41, including post-translational modifications of those sequences. In one embodiment, the antibody comprises the VH sequence of SEQ ID NO:61 and the VL sequence of SEQ ID NO:49, including post-translational modifications of those sequences. In one embodiment, the antibody comprises the VH sequence of SEQ ID NO:62 and the VL sequence of SEQ ID NO:50, including post-translational modifications of those sequences. In one embodiment, the antibody comprises the VH sequence of SEQ ID NO:63 and the VL sequence of SEQ ID NO:51, including post-translational modifications of those sequences. In one embodiment, the antibody comprises the VH sequence of SEQ ID NO:64 and the VL sequence of SEQ ID NO:52, including post-translational modifications of those sequences. In one embodiment, the antibody comprises the VH sequence of SEQ ID NO:65 and the VL sequence of SEQ ID NO:53, including post-translational modifications of those sequences. In one embodiment, the antibody comprises the VH sequence of SEQ ID NO:57 and the VL sequence of SEQ ID NO:40, including post-translational modifications of those sequences.

In a further aspect, the invention provides an antibody that binds to the same epitope as an anti-CRTh2 antibody provided herein. For example, in certain embodiments, an antibody is provided that binds to the same epitope as murine antibody 8B1, 3C12, 31A5, and 19A2, and humanized antibody hu19A2 (including, v1, v12, v38, v46, v47, v51-v53, v57, v58, and v60-v72).

In a further aspect, an anti-CRTh2 antibody is provided that binds to both human CRTh2 and at least one non-human primate CRTh2. In certain embodiments, an anti-CRTh2 antibody binds to human CRTh2 and cynomologous CRTh2. In certain embodiments, an anti-CRTh2 antibody binds to human CRTh2 and rhesus CRTh2. In certain embodiments, an anti-CRTh2 antibody binds to human CRTh2, rhesus CRTh2 and cynomologous CRTh2. In certain embodiments, an anti-CRTh2 antibody binds to both human CRTh2 and at least one non-human primate CRTh2 with a K_{D} of less than 100 nM (e.g., the anti-CRTh2 antibody binds to human CRTh2 with a K_{D} less than 100 nM and binds to at least one non-human primate CRTh2 with a K_{D} of less than 100 nM). In certain embodiments, an anti-CRTh2 antibody binds to both human CRTh2 and at least one non-human primate CRTh2 with a K_{D} of less than 75 nM, 50 nM, 45 nM, 40 nM, 35 nM, 30 nM, 25 nM, 20 nM, 15 nM, or 10 nM. In certain embodiments, an anti-CRTh2 antibody that binds to both human CRTh2 and at least one non-human primate CRTh2 is a depleting antibody, e.g., an antibody that depletes CRTh2 expressing cells as described further herein.

In a further aspect of the invention, an anti-CRTh2 antibody according to any of the above embodiments is a monoclonal antibody, including a chimeric, humanized or human antibody. In one embodiment, an anti-CRTh2 antibody is an antibody fragment, e.g., a Fv, Fab, Fab′, scFv, diabody, or F(ab′)₂ fragment. In another embodiment, the antibody is a full length antibody, e.g., an intact IgG1 antibody or other antibody class or isotype (e.g., IgG₂, IgG₃, or IgG₄) as defined herein. In some embodiments, the antibody comprises the heavy chain sequence selected from the group consisting of SEQ ID NOS:77-83; and/or the light chain sequences selected from the group consisting of SEQ ID NOS:66-76.

In a further aspect, an anti-CRTh2 antibody according to any of the above embodiments may incorporate any of the features, singly or in combination, as described in Sections below:

### Antibody Affinity

In certain embodiments, an antibody provided herein has a dissociation constant (Kd) of ≦1 μM, ≦150 nM, ≦100 nM, ≦50 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays. In some embodiments, Kd may also be measured for binding of antibodies to CRTh2 expressed on cell surface.

According to another embodiment, Kd is measured using surface plasmon resonance assays using a BIACORE®-2000 or a BIACORE®-3000 (BIAcore, Inc., Piscataway, N.J.) at 25° C. with immobilized antigen CMS chips at ˜10 response units (RU). Briefly, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall' Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N. J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N. J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for CRTh2 and the other is for any other antigen. In certain embodiments, bispecific antibodies may bind to two different epitopes of CRTh2. Bispecific antibodies may also be used to localize cytotoxic agents to cells which express CRTh2. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to CRTh2 as well as another, different antigen (see, US 2008/0069820, for example).

### Antibody Variants

In certain embodiments, amino acid sequence variants of the antibodies provided herein are contemplated. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e g, improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided comprising an Fc region wherein a carbohydrate structure attached to the Fc region has reduced fucose or lacks fucose, which may improve ADCC function. Specifically, antibodies are contemplated herein that have reduced fusose relative to the amount of fucose on the same antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In certain embodiments, the antibody is one wherein less than about 50%, 40%, 30%, 20%, 10%, or 5% of the N-linked glycans thereon comprise fucose. For example, the amount of fucose in such an antibody may be from 1% to 80%, from 1% to 65%, from 5% to 65% or from 20% to 40%. In certain embodiments, the antibody is one wherein none of the N-linked glycans thereon comprise fucose, i.e., wherein the antibody is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec 13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, the antibody variants comprising an Fc region described herein are capable of binding to an FcγRIII. In certain embodiments, the antibody variants comprising an Fc region described herein have ADCC activity in the presence of human effector cells or have increased ADCC activity in the presence of human effector cells compared to the otherwise same antibody comprising a human wild-type IgG1Fc region.

### Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues). In an exemplary embodiment, the anti-CRTh2 antibody comprising the following amino acid substitutions in its Fc region: S198A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.)). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

### Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### Recombinant Methods and Compositions

Antibodies may be produced using recombinant methods and compositions, e.g., as described in U.S. Pat. No. 4,816,567. In one embodiment, isolated nucleic acid encoding an anti-CRTh2 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CRTh2 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CRTh2 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N. J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Assays

Anti-CRTh2 antibodies provided herein may be identified, screened for, or characterized for their physical/chemical properties and/or biological activities by various assays known in the art.

### Binding Assays and Other Assays

In one aspect, an antibody of the invention is tested for its antigen binding activity, e.g., by known methods such as ELISA, Western blot, etc.

In another aspect, competition assays may be used to identify an antibody that competes with murine antibody 8B1, 3C12, 31A5, and 19A2, and humanized antibody hu19A2 (including, v1, v12, v38, v46, v47, v51-v53, v57, v58, and v60-v72) for binding to CRTh2. In certain embodiments, such a competing antibody binds to the same epitope (e.g., a linear or a conformational epitope) that is bound by murine antibody 8B1, 3C12, 31A5, and 19A2, and humanized antibody hu19A2 (including, v1, v12, v38, v46, v47, v51-v53, v57, v58, and v60-v72). Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized CRTh2 or cells expressing CRTh2 on cell surface are incubated in a solution comprising a first labeled antibody that binds to CRTh2 (e.g., human or non-human primate) and a second unlabeled antibody that is being tested for its ability to compete with the first antibody for binding to CRTh2. The second antibody may be present in a hybridoma supernatant. As a control, immobilized CRTh2 or cells expressing CRTh2 is incubated in a solution comprising the first labeled antibody but not the second unlabeled antibody. After incubation under conditions permissive for binding of the first antibody to CRTh2, excess unbound antibody is removed, and the amount of label associated with immobilized CRTh2 or cells expressing CRTh2 is measured. If the amount of label associated with immobilized CRTh2 or cells expressing CRTh2 is substantially reduced in the test sample relative to the control sample, then that indicates that the second antibody is competing with the first antibody for binding to CRTh2. See Harlow and Lane (1988) Antibodies: A Laboratory Manual ch.14 (Cold Spring Harbor Laboratory, Cold Spring Harbor, N.Y.).

### Activity Assays

Assays known in the art and described herein (e.g., Example 1) can be used for identifying and testing biological activities of anti-CRTh2 antibodies. In some embodiments, assays for testing anti-CRTh2 antibodies for depleting CRTh2 expressing cells (e.g., Th2 cells, mast cells, eosinophils, basophils, and/or innate type 2 (IT2) cells) are provided. An exemplary test for biological activity may include, e.g., providing transgenic mice expressing human CRTh2 on immune cells, such as basophils and eosinophils, administering an anti-CRTh2 antibody to the transgenic mice, and measuring the level (e.g., number or percentage) of human CRTh2-positive cells in the blood or tissues of mice or the level (e.g., number or percentage) of cell types known to express CRTh2 in the blood or tissues of mice. Another exemplary test may include, e.g., providing mice expressing human CRTh2, sensitizing/challenging the mice with TNP-OVA using known methods, followed by administration of an anti-CRTh2 antibody. TNP-OVA challenged mouse lung tissue, blood, BAL, and BALF may be assessed for the presence of CRTh2-positive cells or the presence of cell types known to express CRTh2. In some embodiments, assays for detecting depletion of Th2 cytokine producing cells by anti-CRTh2 antibodies are provided. For example, in vitro polarized human Th2 cells can be intraperitoneally injected into SCID mice, and an anti-CRTh2 antibody is administered to the mice. The levels of cytokine producing cells may be assessed after ex vivo stimulation with PMA and Ionomycin. In some embodiment, the anti-CRTh2 antibody may deplete at least about any of 50%, 60%, 70%, 80%, 85%, 90%, 95% and 100% of CRTh2 expressing cells in any of these assays.

Assays for testing anti-CRTh2 antibodies for blocking CRTh2 signaling are also provided. An exemplary method for assessing CRTh2 signaling may include providing CRTh2-positive cells, incubating the cells with an anti-CRTh2 antibody, followed by stimulation with a ligand such as PGD2 (in the presence or absence of forskolin), and finally measuring a change in intracellular cAMP or Ca²⁺ content by any method known in the art.

Assays for testing anti-CRTh2 antibodies for preventing recruitment of CRTh2 expressing cells in response to TNP-OVA, papain or prostaglandin D2 are also provided. An exemplary test for recruitment of CRTh2-expressing cells in response to PGD2 may include administration of PGD2 into the airways of a transgenic mice expressing human CRTh2 on immune cells (such as basophils and eosinophils) in the presence or absence of an anti-CRTh2 antibody and assessing the subsequent influx of CRTh2-positive cells into the lung tissue and bronchial alveolar lavage fluid. The assessment may be accomplished in a number of ways including staining excised tissue for CRTh2 and determining cell influx via flow cytometry or any other method known in the art.

Assays for testing anti-CRTh2 antibodies for blocking Ca²⁺ flux in CRTh2 expressing cells are also provided. An exemplary test may include monitoring cells for Ca²⁺ flux using flow cytometry in response to a ligand, such as PGD2, following incubation with indo-1/AM dye and an anti-CRTh2 monoclonal antibody.

### Immunoconjugates

The invention also provides immunoconjugates comprising an anti-CRTh2 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### Methods and Compositions for Diagnostics and Detection

In certain embodiments, any of the anti-CRTh2 antibodies provided herein is useful for detecting the presence of CRTh2 in a biological sample. The term “detecting” as used herein encompasses quantitative or qualitative detection. In certain embodiments, a biological sample comprises a cell or tissue, such as Th2 cells, mast cells, eosinophils, basophils, or innate type 2 (IT2) cells.

In one embodiment, an anti-CRTh2 antibody for use in a method of diagnosis or detection is provided. In a further aspect, a method of detecting the presence of CRTh2 in a biological sample is provided. In certain embodiments, the method comprises contacting the biological sample with an anti-CRTh2 antibody as described herein under conditions permissive for binding of the anti-CRTh2 antibody to CRTh2, and detecting whether a complex is formed between the anti-CRTh2 antibody and CRTh2. Such method may be an in vitro or in vivo method. In one embodiment, an anti-CRTh2 antibody is used to select subjects eligible for therapy with an anti-CRTh2 antibody, e.g. where CRTh2 is a biomarker for selection of patients.

Exemplary disorders that may be diagnosed using an antibody of the invention include asthma, pauci granulocytic asthma, atopic dermatitis, allergic rhinitis, acute or chronic airway hypersensitivity, hypereosinophilic syndrome, eosinophilic esophagitis, Churg-Strauss syndrome, idiopathic pulmonary fibrosis, inflammation associated with a cytokine, inflammation or malignancies associated with CRTh2 expressing cells, chronic idiopathic urticaria, chronic spontaneous urticaria, physical urticarias including cold urticarial and pressure-urticaria, bullous pemphigoid, nasal polyposis, food allergy, and allergic bronchopulmonary aspergillosis (ABPA) with or without concomitant cystic fibrosis.

In certain embodiments, labeled anti-CRTh2 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### Pharmaceutical Formulations

Pharmaceutical formulations of an anti-CRTh2 antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide the following but not limited to the following: an IL4 inhibitor (e.g., AER-001, IL4/IL13 trap, or anti-IL4 antibody), an IL5 inhibitor (e.g., Mepolizumab, CAS No. 196078-29-2; resilizumab, or another anti-IL5 antibody), an IL9 inhibitor (e.g., MEDI-528, or another anti-IL9 antibody), an IL13 inhibitor (e.g., IMA-026, IMA-638 (also referred to as, anrukinzumab, INN No. 910649-32-0; QAX-576; IL4/IL13 trap), tralokinumab (also referred to as CAT-3M, CAS No. 1044515-88-9); AER-001, ABT-308 (also referred to as humanized 13C5.5 antibody), or another anti-IL13 antibody), an anti-IL17 antibody, an anti-IL25 antibody, an anti-IL33 antibody, an anti-TSLP antibody, an anti-OX40L antibody, an anti-OX40 antibody, an IL-4-receptor alpha Inhibitor (e.g., AMG-317, AIR-645, or another anti-IL4Ra antibody), an anti-IL5Ra antibody, an anti-17RA antibody, or an anti-CCR4 antibody. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### Therapeutic Methods and Compositions

Any of the anti-CRTh2 antibodies provided herein may be used in therapeutic methods.

In one aspect, an anti-CRTh2 antibody for use as a medicament is provided. In further aspects, an anti-CRTh2 antibody for use in treating a disorder mediated by CRTh2 is provided. In certain embodiments, an anti-CRTh2 antibody for use in a method of treatment is provided. In certain embodiments, the invention provides an anti-CRTh2 antibody for use in a method of treating an individual having a disorder mediated by CRTh2 comprising administering to the individual an effective amount of the anti-CRTh2 antibody. In one such embodiment, the method further comprises administering to the individual an effective amount of at least one additional therapeutic agent, e.g., as described below. In some embodiments, the disorder is selected from the group consisting of asthma, pauci granulocytic asthma, atopic dermatitis, allergic rhinitis, acute or chronic airway hypersensitivity, hypereosinophilic syndrome, eosinophilic esophagitis, Churg-Strauss syndrome, idiopathic pulmonary fibrosis, inflammation associated with a cytokine, inflammation or malignancies associated with CRTh2 expressing cells, chronic idiopathic urticaria, chronic spontaneous urticaria, physical urticarias including cold urticaria and pressure-urticaria, bullous pemphigoid, nasal polyposis, food allergy, and allergic bronchopulmonary aspergillosis (ABPA) with or without concomitant cystic fibrosis. In further embodiments, the invention provides an anti-CRTh2 antibody for use in depleting CRTh2 expressing cells (e.g., Th2 cells, mast cells, eosinophils, basophils, and/or innate type 2(IT2) cells) in the individual or reducing level of one or more cytokines, enzymes or other inflammatory mediators (e.g., IL-4, IL-5, IL-9, IL-13, IL-17, histamines, tryptase and/or leukotrienes) in the individual. In some embodiments, one or more cytokines produced by at least one of the following cells types is reduced: Th2 cells, mast cells, eosinophils, basophils, or innate type 2(IT2) cells. In certain embodiments, the invention provides an anti-CRTh2 antibody for use in a method of depleting CRTh2 expressing cells (e.g., Th2 cells, mast cells, eosinophils, basophils, and/or innate type 2(IT2) cells) in the individual and/or reducing the level of one or more cytokines, enzymes or other inflammatory mediators (e.g., IL-4, IL-5, IL-9, IL-13, IL-17, histamines, tryptase and/or leukotrienes) in the individual comprising administering to the individual an effective amount of the anti-CRTh2 antibody to deplete CRTh2 expressing cells and/or to reduce one or more cytokines. An “individual” according to any of the above embodiments is preferably a human.

In a further aspect, the invention provides for the use of an anti-CRTh2 antibody in the manufacture or preparation of a medicament. In one embodiment, the medicament is for treatment of a disorder mediated by CRTh2. In a further embodiment, the medicament is for use in a method of treating a disorder mediated by CRTh2 comprising administering to an individual having the disorder an effective amount of the medicament. In one such embodiment, the method further comprises administering to the individual an effective amount of at least one additional therapeutic agent, e.g., as described below. In some embodiments, the disorder is selected from the group consisting of asthma, pauci granulocytic asthma, atopic dermatitis, allergic rhinitis, acute or chronic airway hypersensitivity, hypereosinophilic syndrome, eosinophilic esophagitis, Churg-Strauss syndrome, idiopathic pulmonary fibrosis, inflammation associated with a cytokine, inflammation or malignancies associated with CRTh2 expressing cells, chronic idiopathic urticaria, chronic spontaneous urticaria, physical urticarias including cold urticaria and pressure-urticaria, bullous pemphigoid, nasal polyposis, food allergy and allergic bronchopulmonary aspergillosis (ABPA) with or without concomitant cystic fibrosis. In a further embodiment, the medicament is for depleting CRTh2 expressing cells (e.g., Th2 cells, mast cells, eosinophils, basophils, and/or innate type 2(IT2) cells) in the individual and/or reducing the level of one or more cytokines, enzymes or other inflammatory mediators (e.g., IL-4, IL-5, IL-9, IL-13, IL-17, histamines, tryptase and/or leukotrienes) in the individual. In a further embodiment, the medicament is for use in a method of depleting CRTh2 expressing cells (e.g., Th2 cells, mast cells, eosinophils, basophils, and/or innate type 2(IT2) cells) in the individual and/or reducing level of one or more cytokines, enzymes or other inflammatory mediators (e.g., IL-4, IL-5, IL-9, IL-13, IL-17, histamines, tryptase and/or leukotrienes) in an individual comprising administering to the individual an amount effective of the medicament to deplete CRTh2 expressing cells and/or to reduce one or more cytokines. An “individual” according to any of the above embodiments may be a human.

In a further aspect, the invention provides a method for treating a disorder mediated by CRTh2. In one embodiment, the method comprises administering to an individual having such disorder an effective amount of an anti-CRTh2 antibody. In one such embodiment, the method further comprises administering to the individual an effective amount of at least one additional therapeutic agent, as described below. In some embodiments, the disorder is selected from the group consisting of asthma, pauci granulocytic asthma, atopic dermatitis, allergic rhinitis, acute or chronic airway hypersensitivity, hypereosinophilic syndrome, eosinophilic esophagitis, Churg-Strauss syndrome, idiopathic pulmonary fibrosis, inflammation associated with a cytokine, inflammation or malignancies associated with CRTh2 expressing cells, chronic idiopathic urticaria, chronic spontaneous urticaria, physical urticarias including cold urticaria and pressure-urticaria, bullous pemphigoid, nasal polyposis, food allergy and allergic bronchopulmonary aspergillosis (ABPA) with or without concomitant cystic fibrosis. An “individual” according to any of the above embodiments may be a human.

In a further aspect, the invention provides a method for depleting CRTh2 expressing cells (e.g., Th2 cells, mast cells, eosinophils, basophils, and/or innate type 2(IT2) cells) in the individual and/or reducing level of one or more cytokines, enzymes or other inflammatory mediators (e.g., IL-4, IL-5, IL-9, IL-13, IL-17, histamines, tryptase and/or leukotrienes) in an individual. In one embodiment, the method comprises administering to the individual an effective amount of an anti-CRTh2 antibody to deplete CRTh2 expressing cells and/or reduce one or more cytokines. In one embodiment, an “individual” is a human. In some embodiments, the individual has a disorder selected from the group consisting of asthma, pauci granulocytic asthma, atopic dermatitis, allergic rhinitis, acute or chronic airway hypersensitivity, hypereosinophilic syndrome, eosinophilic esophagitis, Churg-Strauss syndrome, idiopathic pulmonary fibrosis, inflammation associated with a cytokine, inflammation or malignancies associated with CRTh2 expressing cells, chronic idiopathic urticaria, chronic spontaneous urticaria, physical urticarias including cold urticaria and pressure-urticaria, bullous pemphigoid, nasal polyposis, food allergy and allergic bronchopulmonary aspergillosis (ABPA) with or without concomitant cystic fibrosis.

In certain embodiments, the methods described herein may be used to treat an individual suffering from asthma, wherein the individual is eosinophilic inflammation positive (EIP) as defined in US 2012/0156194. In certain embodiments, the methods described herein may be used to treat an individual suffering from asthma, wherein the individual is eosinophilic inflammation negative (EIN) as defined in US 2012/0156194. See also, D F Choy et al., J Immunol. 186(3): 1861-9 (2011); Arron et al. (2013) Adv Pharmacol 66: 1-49; and G. Jia et al., J Allergy Clin Immunol. 130(3): 647-654 (2012).

In certain embodiments, patients suffering from asthma show a high level of total serum or plasma periostin. In certain embodiments, an EIP patient refers to a patient who has been tested for serum or plasma periostin levels, wherein the serum or plasma periostin level is equal to or more than the medium or mean serum or plasma periostin level of a patient population (may also be referred to as high periostin). In certain embodiments, the patient who has been tested for serum or plasma periostin levels using, for example, an ELISA or a sandwich immunoassay as described herein, would have Total Periostin levels of 20 ng/ml or higher (Eosinophilic Positive). According to certain embodiments, the Total Periostin levels in a patient who is EIP can be selected from the group consisting of 21 ng/ml or higher, 22 ng/ml or higher, 23 ng/ml or higher, 24 ng/ml or higher, 25 ng/ml or higher, 26 ng/ml or higher, 27 ng/ml or higher, 28 ng/ml or higher, 29 ng/ml or higher, 30 ng/ml or higher, 31 ng/ml or higher, 32 ng/ml or higher, 33 ng/ml or higher, 34 ng/ml or higher, 35 ng/ml or higher, 36 ng/ml or higher, 37 ng/ml or higher, 38 ng/ml or higher, 39 ng/ml or higher, 40 ng/ml or higher, 41 ng/ml or higher, 42 ng/ml or higher, 43 ng/ml or higher, 44 ng/ml or higher, 45 ng/ml or higher, 46 ng/ml or higher, 47 ng/ml or higher, 48 ng/ml or higher, 49 ng/ml or higher, 50 ng/ml or higher, 51 ng/ml or higher, 52 ng/ml or higher, 53 ng/ml or higher, 54 ng/ml or higher, 55 ng/ml or higher, 56 ng/ml or higher, 57 ng/ml or higher, 58 ng/ml or higher, 59 ng/ml or higher, 60 ng/ml or higher, 61 ng/ml or higher, 62 ng/ml or higher, 63 ng/ml or higher, 64 ng/ml or higher, 65 ng/ml or higher, 66 ng/ml or higher, 67 ng/ml or higher, 68 ng/ml or higher, 69 ng/ml or higher and 70 ng/ml or higher in the serum or plasma.

In certain embodiments, patients suffering from asthma show a low level of total serum or plasma periostin. In certain embodiments, an EIN patient refers to a patient who has been tested for serum or plasma periostin levels, wherein the serum or plasma periostin level less than 20 ng/ml.

It should be understood that the EIP Status represents the state of the patient, and is not dependent on the type of assay used to determine the status. Thus, other Eosinophilic Inflammation Diagnostic Assays, including other periostin assays such as the ELISA assay and the ELECSYS® periostin assays shown in US2012/0156194, can be used or developed to be used to test for Eosinophilic Inflammation Status and measure Total Periostin levels. See also Jia et al., 2012, J. Allergy Clin. Immunol. 130:647-654, and US2012/0156194, which are hereby incorporated by reference in their entireties.

The term “Total Periostin” as used herein refers to at least isoforms 1, 2, 3 and 4 of periostin. Human periostin isoforms 1, 2, 3 and 4 are known in the art as comprising the following amino acid sequences: NP_006466 (SEQ ID NO:87); NP_001129406 (SEQ ID NO:88), NP_001129407 (SEQ ID NO:89), and NP_001129408 (SEQ ID NO:90), respectively, according to the NCBI database, and isoform 5 and has been partially sequenced. Isoform 5 comprises the amino acid sequence of SEQ ID NO:91. In one embodiment, the isoforms of periostin are human periostins. In a further embodiment, the term Total Periostin includes isoform 5 of human periostin in addition to isoforms 1-4. In another embodiment, Total Periostin is Total Serum Periostin or Total Plasma Periostin (i.e., Total Periostin from a serum sample obtained from whole blood or a plasma sample obtained from whole blood, respectively, the whole blood obtained from a patient).

In some embodiments, the anti-CRTh2 antibody administered to the individual depletes CRTh2 expressing cells in the individual. In some embodiments, the antibody depletes CRTh2 expressing cells from lung tissue and/or from bronchoalveolar lavage fluid. In some embodiments, at least one type of CRTh2 expressing cells (such as from lung) in the individual is depleted by at least about any of 50%, 60%, 70%, 80%, 85%, 90%, 95% and 100% as compared to a baseline before administering the antibody. In some embodiments, at least one type of cytokine Th2 producing cells (such as from lung) in the individual is depleted by at least about any of 50%, 60%, 70%, 80%, 85%, 90%, 95% and 100% as compared to a baseline before administering the antibody. As used herein, a “baseline” refers to a level before an administration of an anti-CRTh2 antibody described herein to the individual. The level of CRTh2 expressing cells before and after administration of the antibody can be tested using methods known in the art and described herein.

In a further aspect, the invention provides pharmaceutical formulations comprising any of the anti-CRTh2 antibodies provided herein, e.g., for use in any of the above therapeutic methods. In one embodiment, a pharmaceutical formulation comprises any of the anti-CRTh2 antibodies provided herein and a pharmaceutically acceptable carrier. In another embodiment, a pharmaceutical formulation comprises any of the anti-CRTh2 antibodies provided herein and at least one additional therapeutic agent, e.g., as described below.

Antibodies of the invention can be used either alone or in combination with other agents in a therapy. For instance, an antibody of the invention may be co-administered with at least one additional therapeutic agent. In certain embodiments, an additional therapeutic agent is an inhaled corticosteroid, a short acting (32 agonist, a long acting (32 agonist, a long acting muscarinic agonist, a leukotriene receptor antagonist, a mast cell inhibitor (such as, for example, cromolyn), a CRTh2 small molecule inhibitor, or a combination thereof.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using an immunoconjugate of the invention in place of or in addition to an anti-CRTh2 antibody.

### Articles of Manufacture and Kits

In another aspect of the invention, an article of manufacture or a kit comprising one or more of the anti-CRTh2 antibodies useful for the treatment, prevention and/or diagnosis of the disorders described above is provided. The article of manufacture or kit may further comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture or kit may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture or kit in this embodiment of the invention may further comprise a package insert indicating that the compositions can be used to treat a particular condition. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture or kit may include an immunoconjugate of the invention in place of or in addition to an anti-CRTh2 antibody.

### EXAMPLES

The following are examples of methods and compositions of the invention. It is understood that various other embodiments may be practiced, given the general description provided above.

### Example 1

### Generation of Murine Anti-Human CRTh2 Antibodies

### Materials and Methods

Cloning and Cell Lines

Rhesus and cyno CRTh2 cDNA were obtained by RT-PCR from total RNA extracted from rhesus and cyno blood and cloned into mammalian expression vector pRK5 vector containing an amino-terminal Flag tag, a gD tag or no tag. Human full-length cDNA from Origene (Gene Bank NM_004778) was cloned into vector pRK5 with an amino-terminal Flag tag, a gD tag or no tag Upon sequence confirmation, the CRTh2 clone contained an alanine at position 204 rather than a valine as indicated in Gene Bank NM_004778 (e.g., SEQ ID NO: 84 which has a V204A substitution relative to the Gene Bank reference sequence).

CRTh2-containing plasmids were transfected into 293 cells using Fugene 6 (Roche) and surface expression of tagged or untagged CRTh2 was confirmed with monoclonal anti-Flag antibody (clone M2, Sigma), anti-gD Ab (clone 952, Genentech) or with specific anti-CRTh2 Abs including rat anti-CRTh2 antibody BM16 (BD Pharmingen) against human CRTh2. CRTh2-containing plasmids were also introduced into 300.19 cells, a mouse pre-B cell line, by electroporation and surface CRTh2 expression was confirmed with Flag-tag expression. CRTh2 expression on the surface of 300.19 cells was also determined by anti-CRTh2 monoclonal antibodies including clone BM16 (BD Pharmingen) against human CRTh2.

### Generation of Anti-Human CRTh2 Antibodies

To generate anti-human CRTh2 antibodies, Balb/c mice (Charles River) were immunized with one of the two methods: DNA immunization and cell immunization. For DNA immunization, Balb/c mice were immunized weekly by hydrodynamic tail vein injection with 50 ug of human CRTh2 DNA in pRK5 vector plus mouse Flt3-L and GM-CSF as adjuvants. For cell immunization, Balb/c mice (Charles River, Hollister, Calif.) were immunized intraperitoneally with 5 million 300.19 cells stably transfected with human CRTh2 diluted in PBS twice weekly via i.p. injection. Mice received 10 doses, followed by a pre-fusion boost of 20 million cells i.v. along with 40 million cells i.p., three days prior to fusion.

Hybridomas were generated by standard methods. Splenocytes were fused with X63-Ag8.653 mouse myeloma cells (American Type Culture Collection, Rockville, Md.) via electrofusion (BTX, Hawthorne, N.Y.) and incubated at 37° C., 7% CO2, overnight in Dulbecco's Modified Eagle's Medium (DMEM; Lonza, Basel, Switzerland) supplemented with 10% Fetal bovine serum (FBS), 4.5 g/L glucose, 25 mM HEPES, 0.15 mg/ml oxaloacetic acid, 100 μg/ml pyruvic acid, 0.2 U/ml insulin, 2 mM L-glutamine, 100 U/ml penicillin, 100 μg/ml streptomycin (Penicillin-Streptomycin, Invitrogen, Carlsbad, Calif.), NCTC-109 (Lonza), NEAA (Invitrogen), before plating into 96-well plates in media as described supplemented with 5.7 μM azaserine and 100 μM hypoxanthine (HA, Sigma-Aldrich, St. Louis, Mo.). Cells were cultured for 10 days, followed by ELISA and FACS analyses. Cells from wells demonstrating expression of mouse IgG and showing strong specific binding by FACS were expanded and subcloned by limiting dilution. Final clones demonstrating the highest FACS binding after the second round of subcloning were expanded for large-scale production in bioreactors (Integra Biosciences, Chur, Switzerland). Supernatants were then purified by Protein A affinity chromatography as previously described (Hongo et al., Hybridoma 19:303, 2000). Purified antibodies from hybridomas were screened by flow cytometry for the ability to bind human CRTh2 expressed on 293 cells or 300.19 cells. Binding reactivity was also tested on basophils and eosinophils from human peripheral blood. The light and heavy chain of clones 19A2, 8B1, 31A5 and 3C12 were subcloned into pRK5 vectors. All heavy chains were cloned to contain the mouse IgG2a Fc region. Anti-CRTh2 antibodies were produced in CHO cells using standard procedures. Afucosylated 19A2 and 8B1 antibodies were produced from a FUT8^{-/-} CHO cell line.

Flow Cytometry

Human whole blood was obtained from healthy donors and peripheral blood mononuclear cells (PBMC) were used for staining procedures after red blood cell lyses with EL buffer (Qiagen). Blood cells were incubated with A467-conjugated anti-CRTh2 antibodies at various concentrations or with anti-CRTh2 antibodies plus secondary anti-mouse IgG-PE antibodies (Jackson ImmunoResearch Laboratory). Antibodies used for staining leukocyte populations were as follows: FITC-anti-human CD15, CD16, PerCP-anti-human HLADR and CD4, APC-anti-human CD123, CXCR3, CD14, BDCA1, biotin-anti-human CCR6, and PE-anti-human CCR4. Abs used were purchased from BD Pharmingen. To determine CRTh2 expression on regulatory T cells, CD4+CD25+ T cells were enriched from human PBMCs by MACS isolation (Miltenyi Biotec), surface stained with anti-CRTh2 (antibody BM16), followed by intracellular staining with anti-FoxP3 (BD Bioscience). To assess CRTh2 expression on human mast cells, mast cells were generated by culturing fresh human bone marrow CD34+ cells (AllCells) in StemPro-34 SFM complete medium (Gibco) with 200 ng/mL rhIL-6, 100 ng/mL rhSCF (PeproTech), and 30 ng/mL rhIL-3 (R&D system) for 3-4 weeks. Mast cells were stained with anti-CD117, anti-CD123, and anti-FceRI (BD Bioscience). To determine human CRTh2 expression on Th2 cells in human CRTh2.Bac.Tg mice, 50 ug papain in 50 ul PBS was injected into the mouse right hind footpad, popiteal lymph node cells were collected three days later and stained with anti-mCD4-PerCP, anti-mCD44-FITC, and BM16-A647. To examine human CRTh2 expression on innate T helper type (IT) 2 cells in human CRTh2.Bac.Tg mice, 50 ug mouse IL-17E in pRK5 vector was injected into the tail vein hydrodynamically. Three days later mesentery lymph node cells were collected and stained with anti-mCD117-PE, BM16-A647, propidium iodide and Lineage markers (FITC-labeled: CD3, CD4, CD8, B220, FceRI, CD11c, Gr1, NK1.1, F4/80, DX5 and PerCP-labeled: CCR3). FITC-anti-human CD15, CD16, PerCP-anti-human HLADR and CD4, APC-anti-human CD123, CXCR3, CD14, BDCA1, biotin-anti-human CCR6, and PE-anti-human CCR4 were purchased from BD Pharmingen. Cynomolgus monkey and rhesus monkey blood was obtained from healthy monkeys and peripheral blood mononuclear cells (PBMC) were used for staining procedures after red blood cell lyses with EL buffer (Qiagen). Blood cells were incubated with A467-conjugated anti-CRTh2 antibodies at various concentrations. Antibodies used for staining leukocyte populations were as follows: FITC-anti-human CD123 (BD Pharmingen), PE-anti-human CD125 (BD Pharmingen), and PerCP-eFluor710 anti-human FceRI (eBioscience). Samples were acquired on a FACSCalibur flow cytometer using CellQuest Pro software (BD Biociences) and data analysis was conducted using Flowjo (Tree Star, Inc).

CRTh2+ Memory CD4+ T Cells Isolation and Quantitation of Cytokine Production

Untouched memory CD4+ T cells were isolated from human PBMCs from an atopic donor by MACS isolation (Miltenyi Biotec), followed by staining with CD45RO-FITC, CD4-PerCP (BD Pharmingen), and BM16-PE (Miltenyi Biotec) antibodies at 37° C. for 20 min CRTh2+CD45RO+CD4+ and CRTh2-CD45RO+CD4+ memory T cells were sorted by FacsAria sorter (BD). Purities of CRTh2+ and CRTh2− memory CD4+ T cells were above 98%. The same numbers of sorted cells were stimulated with 10 ug/mL of plate-bound anti-hCD3 mAb and 1 ug/mL soluble anti-hCD28 for 48 hrs at 37° C. Supernatants were collected and analyzed for IL-4, IL-5, IL-9, IL-13, IL-17A, TNFα, IFNγ, and GM-CSF using human Bio-Plex (Bio-Rad) antibody-immobilized beads and plate read using Luminex 100 instrument (Luminex) according to manufacturer's protocol.

Radioligand Cell Binding Assay (Scatchard Analysis)

The equilibrium dissociation constants (K_{D}) for anti-CRTH2 antibodies binding to cells expressing recombinant CRTh2 receptor were determined using a radioligand cell binding assay. The anti-CRTh2 antibodies were iodinated using the Iodogen method and the radiolabeled antibodies had a range of specific activities of 19-22 μCI/μg for the Fab antibodies and 10-14 μCI/μg for the IgG antibodies. The cells expressing the CRTh2 receptor were incubated for 2 hours at room temperature with a fixed concentration of iodinated anti-CRTh2 antibody combined with increasing concentrations of unlabeled anti-CRTh2 antibody and including a zero-added, buffer only sample. After the 2-hour incubation, the competition reactions were transferred to a Millipore Multiscreen filter plate and washed 4 times with binding buffer to separate the free from bound iodinated antibody. The filters were counted on a Wallac Wizard 1470 gamma counter. The binding data was evaluated using NewLigand software (Genentech), which uses the fitting algorithm of Munson and Rodbard to determine the binding affinity of the anti-CRTh2 antibody (Munson and Rodbard, Anal. Biochem, 1980; 107: 220-239).

Epitope Mapping

Purified mouse anti-CRTh2 monoclonal antibody 19A2 and rat anti-CRTh2 monoclonal antibody BM16 are biotinylated using the EZ-Link Sulfo-NHS-Biotin kit (Pierce/Thermo-Fisher, Rockford, Ill.). Activity is confirmed by FACS titration on 293 cells stably transfected with human CRTh2 or rhesus CRTh2. 50 ul of transfected 293 cells are added to 96-well U-bottom plates (BD Falcon, Franklin Lakes, N.J.) suspended in PBS containing 1% FBS at a concentration of 10 million cells/ml, followed by 50 μl of unlabeled antibodies at a concentration of 20 ug/ml, and plates are incubated for 30 minutes at 4° C. Biotinylated antibodies are added to the plate at a concentration of 2 ug/ml (19A2 and BM16) as determined by previous FACS titration experiments, and plates are incubated for 30 minutes at 4° C. Cells are washed twice using centrifugation to pellet cells followed by addition of 200 μl of PBS containing 1% FBS. Cells are then incubated with phycoerythrin-conjugated steptavidin (Zymed/Life Technologies, Grand Island, N.Y.) for 30 minutes at 4° C. Cells are washed, fixed in PBS containing 1% formalin, and analyzed by flow cytometry on a FACSCalibur flow cytometer (BD Biosciences, San Jose, Calif.).

Human T Cell Polarization

Untouched naïve CD4+ T cells were isolated from PBMCs from a healthy donor by MACS separation (Miltenyi Biotec). Cells were cultured in complete DMEM media supplemented with 10% FBS, 2 mM L-glutamine, 50 uM 2-ME, 1 mM sodium pyruvate, 100 U/mL penicillin, 100 ug/mL streptomycin, and 1 mM non-essential amino acid in the presence of 10 ug/mL of plate-bound anti-CD3 mAb and 1 ug/mL soluble anti-CD28 mAb (BD Biociences). Human Th subset polarization conditions were as follows: Th1: 10 ng/mL rhIL12 and 2 ug/mL anti-hIL-4; Th2: 20 ng/mL rhIL-4 (R&D System), 2 ug/mL anti-hIL-12 and 5 ug/mL anti-hIFNγ (BD Biosciences). Two rounds of polarization were performed with each round consisting of an activation phase followed by a rest phase and with the second restimulation performed in the presence of 1 ug/mL plate-bound anti-CD3 mAb and 1 ug/ml soluble anti-CD28 mAb.

Calcium Mobilization Assay

In vitro polarized Th2 Cells were incubated with 5 μM indo-1/AM and 0.2% pluronic F127 (Molecular Probe) at 37° C. for 30 min, washed, and subsequently stained with anti-CCR6, anti-CCR4, anti-CD4, and anti-CXCR3 mAbs at 37° C. for 15 min; after washing the cells were incubated with 1 uM of anti-CRTh2 mAbs or isotype control antibodies at 37° C. for 30 min, and then stimulated with 100 nM PGD2 (Sigma). Calcium release was monitored by flow cytometry.

CRTh2 Functional cAMP Blocking Assay

The blocking activities of anti-CRTh₂ antibodies in PGD₂-CRTh₂ signaling were analyzed by measuring the cellular cAMP levels after the incubation of anti-CRTh₂ with a recombinant cell line cAMP Hunter™ CHO-K₁ CRTh₂ Gi (DiscoveRx; Fremont, Calif.), in the presence of Forskolin and PGD₂ (Sigma; St. Louis, Mo.). Briefly, the cells were cultured overnight at 10,000 cells/well in a 384-well tissue culture plate (Corning Cat#3707; Corning, N.Y.). After removal of the culture medium, 10 μL of testing antibody (serially diluted in PBS) were added to each well and incubated for 30 min at 37° C. Then Forskolin and PGD₂ were added to each well to reach final concentrations of 12.5 μM and 4 nM respectively. After another 30 min incubation at 37° C., the plate was subjected to cAMP analysis using HitHunter® cAMP XS+ kit (DiscoveRx) according to the manufacturer's protocol, with Envison (Perkin Elmer; Waltham, Mass.) as the chemiluminescence reader. The data were then analyzed and plotted using Prism (GraphPad Software; La Jolla, Calif.).

Generation of Human CRTh2 BAC Transgenic Mice

A 171 Kb fragment containing the human CRTh2 gene in a BAC vector backbone (RPCI human BAC library 11; clone ID RP11-68H20) was purchased from Invitrogen. A shorter version of 28 Kb was obtained through recombineering. The 171 Kb or 28 Kb BAC constructs were microinjected into fertilized oocytes harvested from C57BL/6 mice. The presence of the human CRTh2 transgene was determined by RT-PCR from mouse tail DNA. Of nine founders identified, seven gave rise to similar human CRTh2 expression patterns on immune cell types tested by flow cytometry. Of these lines, line 85 demonstrated by flow cytometry similar human CRTh2 expression levels on mouse basophils and eosinophils when compared to human CRTh2 levels on primary human basophils and eosinophils from human PBMC.

Measurement of Blood Basophil and Eosinophil Depletion with Anti-CRTh2 Antibodies in Human CRTh2 BAC Transgenic Mice

Mouse or humanized anti-human CRTh2 antibodies or isotype control antibodies (mIgG1: ant-gp120 antibodies; mIgG2a: anti-ragweed antibodies; hIgG1: anti-gD antibodies) were intravenously injected on day 0 into 6-8 week old human CRTh2 BAC tg mice at doses indicated. Eye bleeds were taken after 3, 6 or 7 days to analyze basophil and eosinophil numbers by flow cytometry as indicated. Alternatively, a group of mice were sacrificed on day 2, 3, 7 or 14 and blood, spleen and bone marrow were harvested and processed for enumeration of eosinophils and basophils by flow cytometry. Red blood cells were lysed with EL buffer (Qiagen). White blood cells, splenocytes or bone marrow cells were stained with anti-CD123-FITC, anti-FcεRI-PE, and anti-CCR3-PerCP to detect basophils and eosinophils. Absolute cell number was determined by flow cytometry using CaliBRITE FITC beads (BD Biosciences).

TNP-OVA Induced Lung Inflammation in Human CRTh2 BAC Transgenic Mice

Human CRTh2 BAC tg mice were sensitized on Day 0 by intraperitoneal injection with 50 ug TNP-OVA (Biosearch Technologies) in 2 mg aluminum hydroxide in 100 ul sterile PBS. Starting on Day 35 post sensitization, mice were challenged for seven consecutive days with aerosolized 1% TNP-OVA in PBS for 30 min via a nebulizer. Mice were treated intraperitoneally with 200 ug of anti-human CRTh2 antibodies clone 19A2 mIgG2a (afucosylated or wt), the Fc mutant Ab 19A2 mIgG2a_DANA, or anti-ragweed control antibody (mIgG2a) in 100 uL of saline once per day on day 38 to 41. All mice were euthanized on day 42. Mice were perfused through the right ventricle with 20 ml of PBS to clear the lungs of peripheral blood, and the entire lung was removed for flow cytometry. Blood was collected via cardiac puncture for evaluation by flow cytometry. BAL was collected for cell count and cytokine analyses. The BAL fluid IL-4 and IL-13 cytokine concentrations were determined by ELISA (R&D) according to the manufacturer's protocol. An effector function-deficient version of the 19A2 antibody, 19A2_DANA, was created by mutating 2 residues (D265A, N297A), which abrogated Fcγ receptor binding.

Human SCID Model to Assess the Potential of Anti-Human CRTh2 Abs to Deplete Th2 Cells

On Day −7, human PBMCs were isolated by leukopheresis and Ficoll density gradient centrifugation (GE Healthcare) from an atopic donor with serum IgE level of 315 ng/mL. Aliquots of PBMCs from the same donor were frozen down for transfer into mice later. Untouched naïve CD4+ T helper cells were further isolated from PBMCs by depletion of non-CD4+ T cells and memory T cells using naïve CD4+ T cell isolation kit II (Miltenyi Biotec 130-094-131). Purified naïve CD4+ T cells were stimulated with plate-bound anti-CD3 at 10 ug/mL (BD 555329) and 1 ug/mL of soluble anti-CD28 (BD 555725) for three days under skewing condition towards Th2: 5 ug/mL of anti-human IFNg (BD 554698), 2 ug/mL of anti-IL12 (BD 554659), and 20 ng/mL of recombinant human IL-4 (R&D System 204-IL). The CD4+ T cells stimulated under Th2 conditions were used for transfer experiments into SCID-beige mice.

On day 0, SCID-beige mice (Charles River) were irradiated sublethally with 3.5 Gy from a cesium 137 source. Human T cells were transferred in 100 ul of PBS via intraperitoneal injection into mice in the following mixture: 6×10⁷ of polarized T cells (as described above) and 4×10⁷ of live previously frozen human PBMCs from the same donor. 100 ug of anti-human IFNg and 100 ug of anti-human IL-12 antibodies in 100 ul of PBS were intraperitoneally injected into mice on day 0 and day 3. 100 ng of recombinant human IL-4 was intraperitoneally injected into mice on day 1, 2 and day 3. Mice were treated with 200 ug of anti-human CRTh2 antibody (clone 19A2, afucosylated) or anti-ragweed isotype control antibody in 100 uL of PBS on day 0 before cell transfer and on day 3. All mice were euthanized on day 7 and spleens were collected. Splenocytes were stimulated ex vivo with PdBu (50 ng/mL) and ionomycin (500 ng/mL) at 37° C. for 4.5 hours for assessment of intracellular cytokine levels by FACS. Cells were surface stained with anti-hCD4 and stained with anti-mCD45, anti-mTer119, and anti-hCD19 in the same channel to exclude these lineage positive cells. Cells were fixed and stained with anti-hIFNg and anti-hIL-4

Model for Depletion of IT2 Cells

To increase the number of innate T helper type (IT) 2 cells in human CRTh2.Bac.Tg mice, 50 ug/mouse IL-17E in pRK5 vector was injected in 1.6 ml Ringer's solution hydrodynamically into the tail vein. Three days later mesentery lymph node cells were collected and IT2 cell percentage and numbers were determined by flow cytometry by staining with anti-mCD117-PE, BM16-A647, and excluding lineage positive as well as dead cells (lin: CD3, CD4, CD8, B220, FceRI, CD11c, Gr1, NK1.1, F4/80, DX5 and CCR3).

### Results

CRTh2 is Expressed on Cells Associated with Asthma

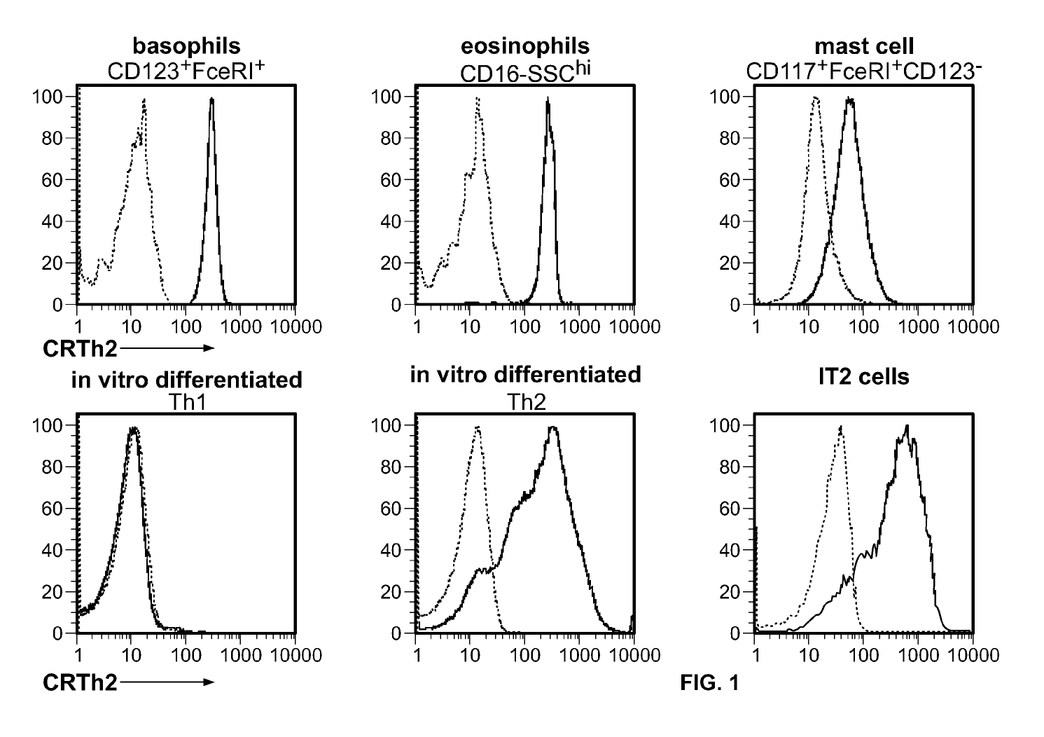

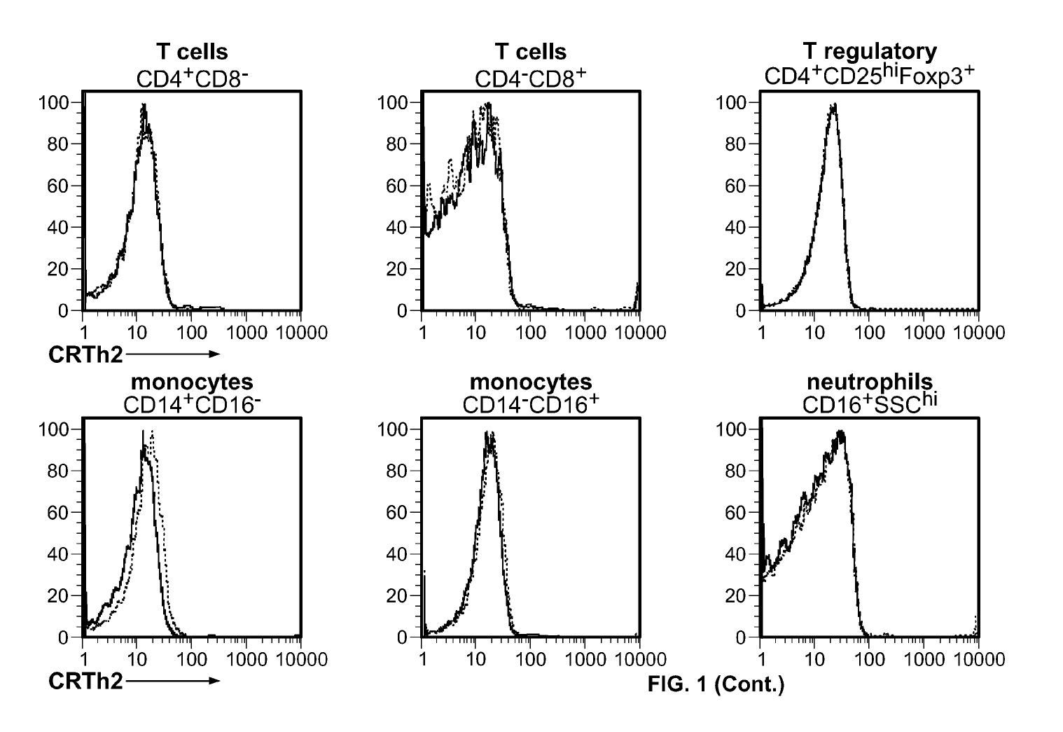

CRTh2 expression on cells from human PBMCs or cultured human cells was assessed by flow cytometry with anti-human CRTh2 antibody (clone BM16) (FIG. 1). CRTh2 is selectively expressed on human blood basophils, eosinophils, polarized Th2 cells, bone marrow derived mast cells as well as on innate T helper type 2 (IT2) cells as recently reported (Mjosberg, Nat. Imm 12(11):1055-62 (2011)). CRTh2 is not expressed on polarized Th1 cells, neutrophils, dendritic cells, monocytes, and regulatory T cells. CRTh2 expression is not detected on B cell, NK cells, NK T cells, and platelets (data not shown).

CRTh2 Cells are Associated with Th2 Cytokine Production

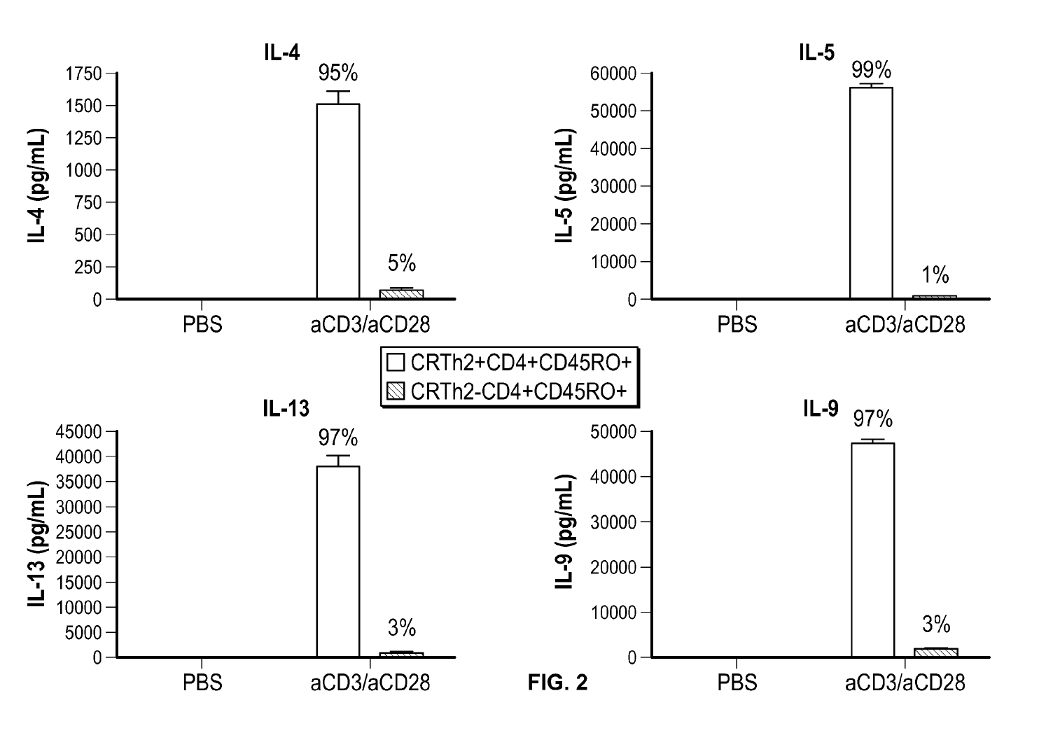

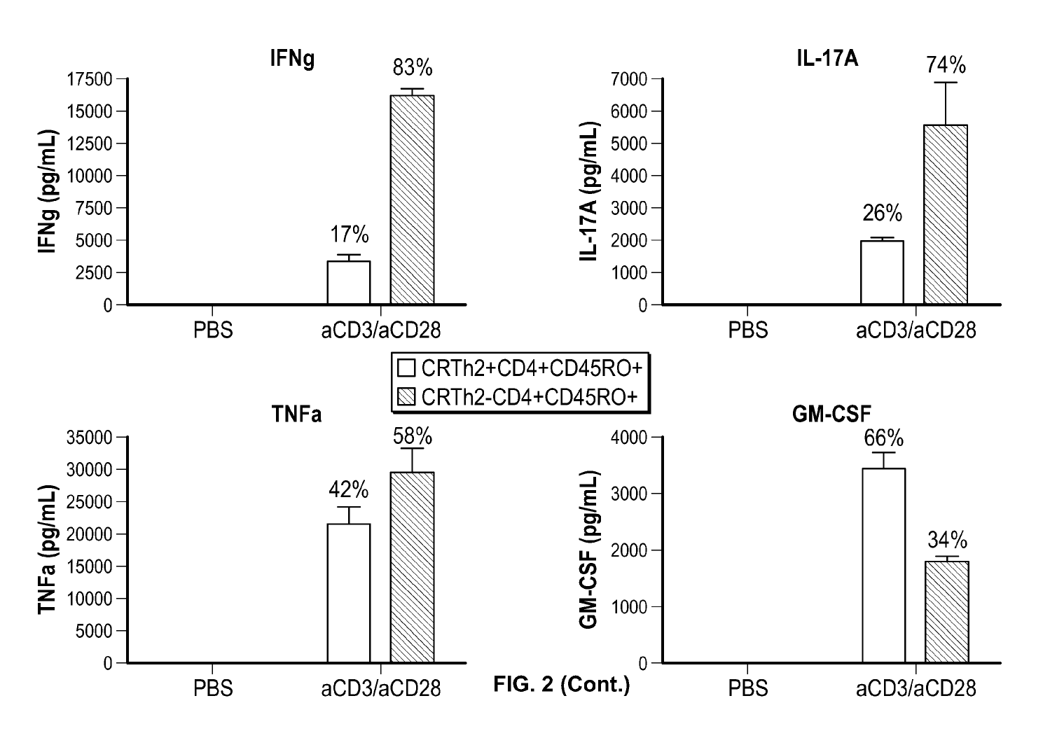

To assess that CRTh2 is expressed on T cell subsets that are associated with Th2 cytokine production, CRTh2+ and CRTh2− memory CD4 T cells were FACS sorted and stimulated with anti-CD3 and anti-CD28 antibodies to assess Th2 cytokine production. CRTh2+ memory CD4 T cells produced more than 95% of memory T cell Th2 cytokines when compared to the CRTh2− memory CD4 T cell populations (FIG. 2). Additional donors tested showed similar results.

Generation and In Vitro Characterization of Anti-Human CRTh2 Antibodies

Anti-human CRTh2 antibodies were generated from Balb/c mice immunized without adjuvant with 300.19 cells overexpressing human CRTh2.

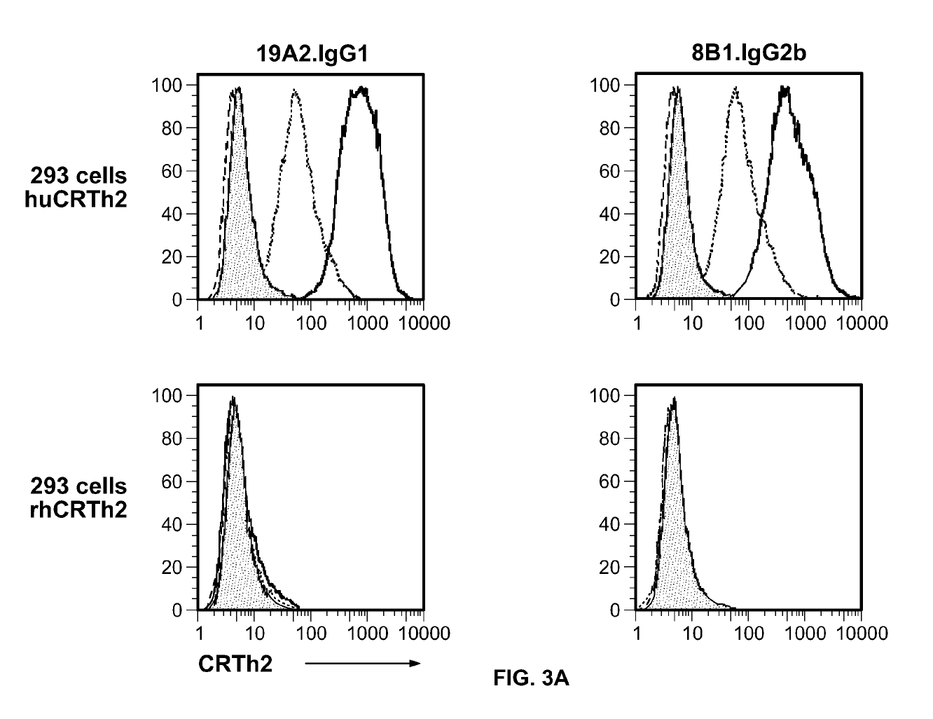

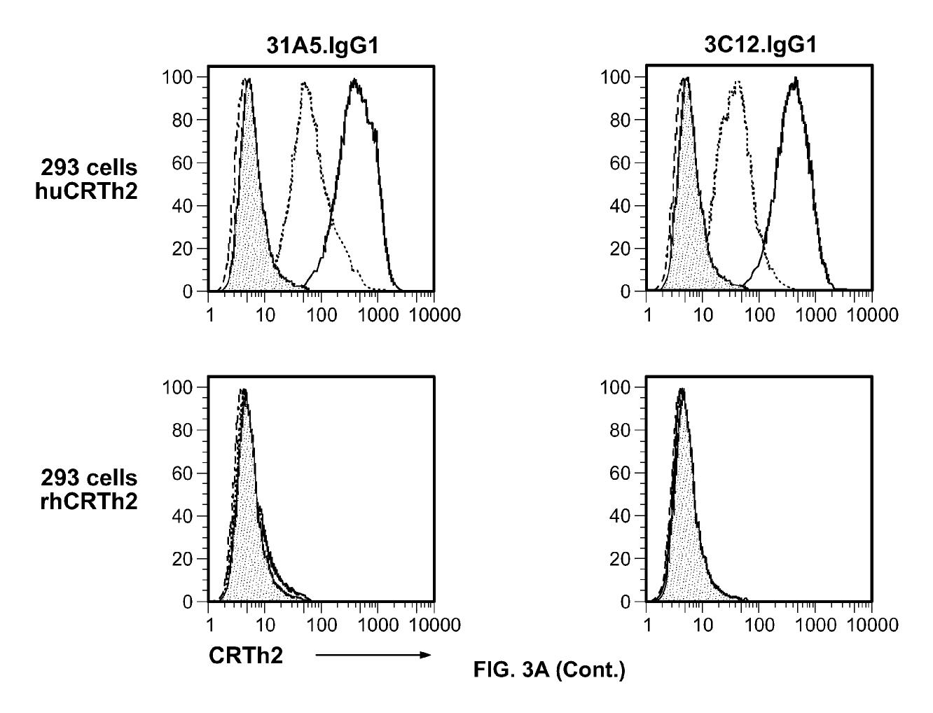

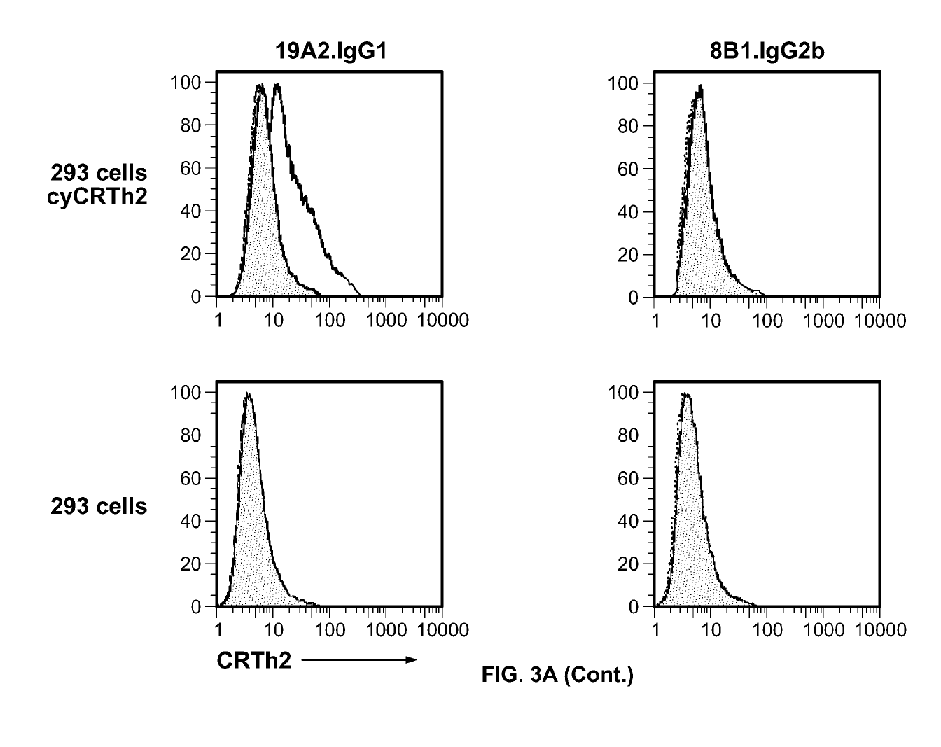

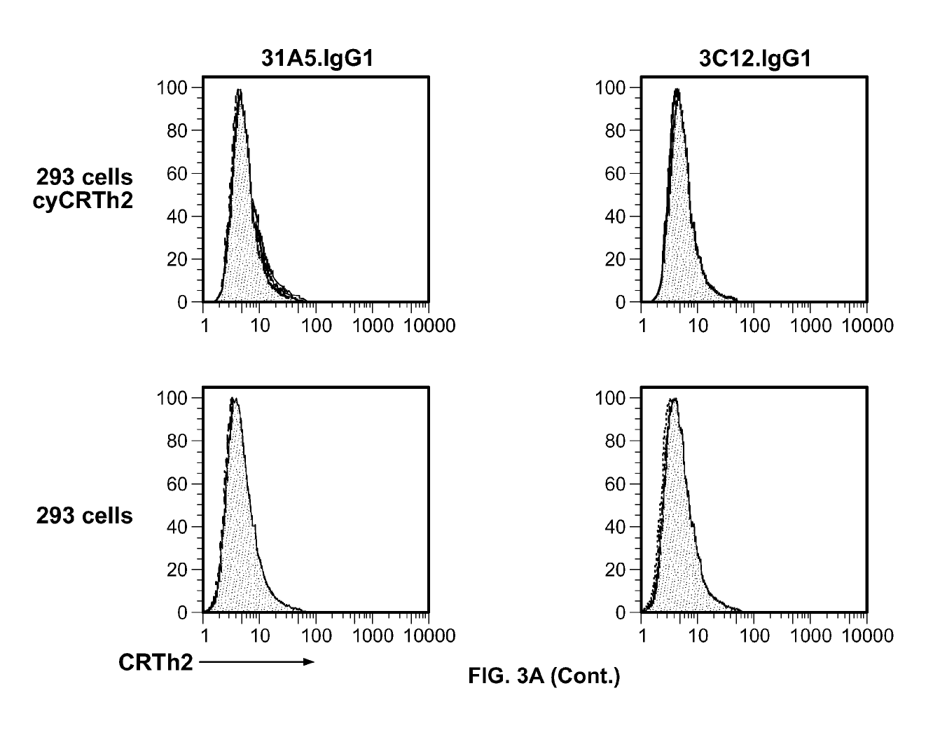

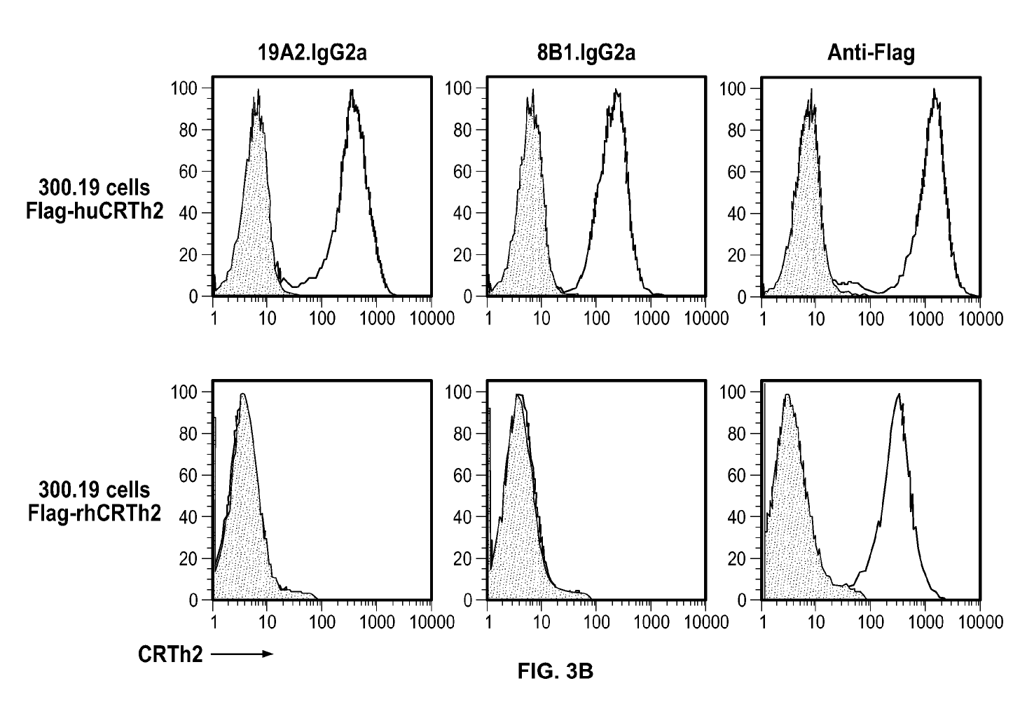

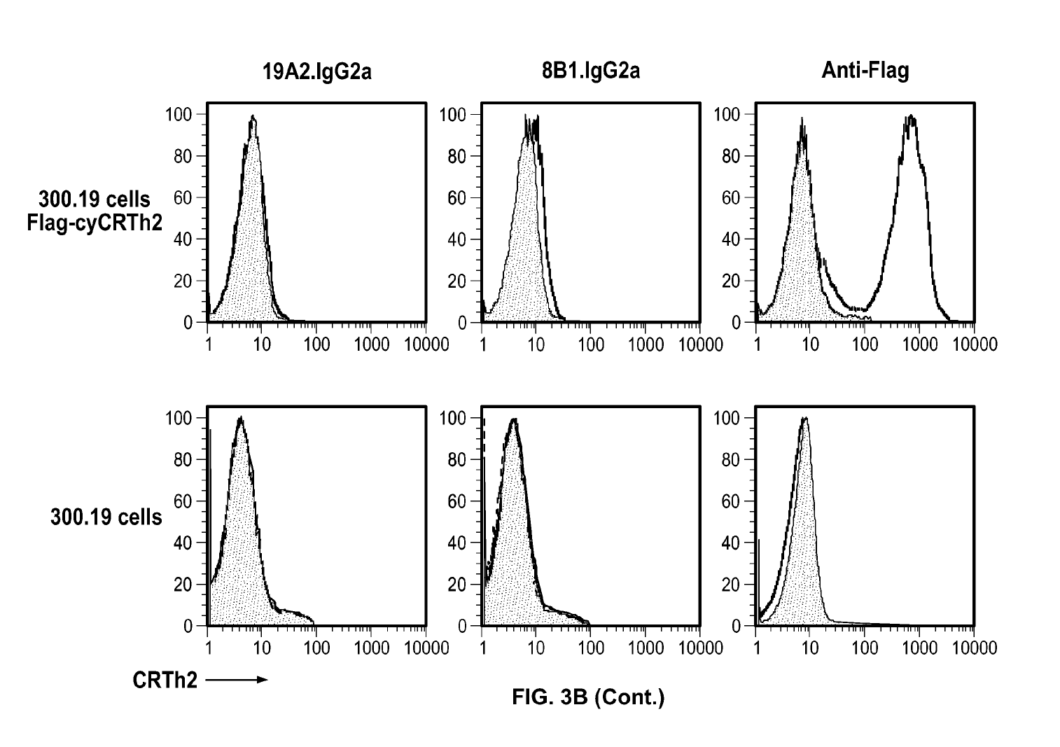

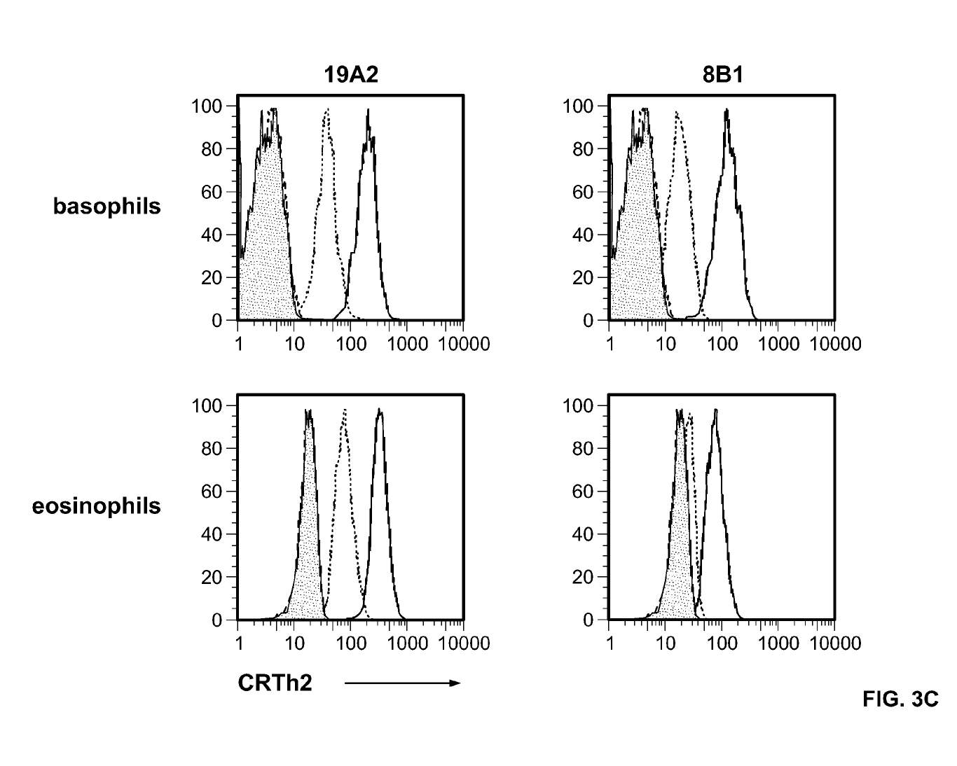

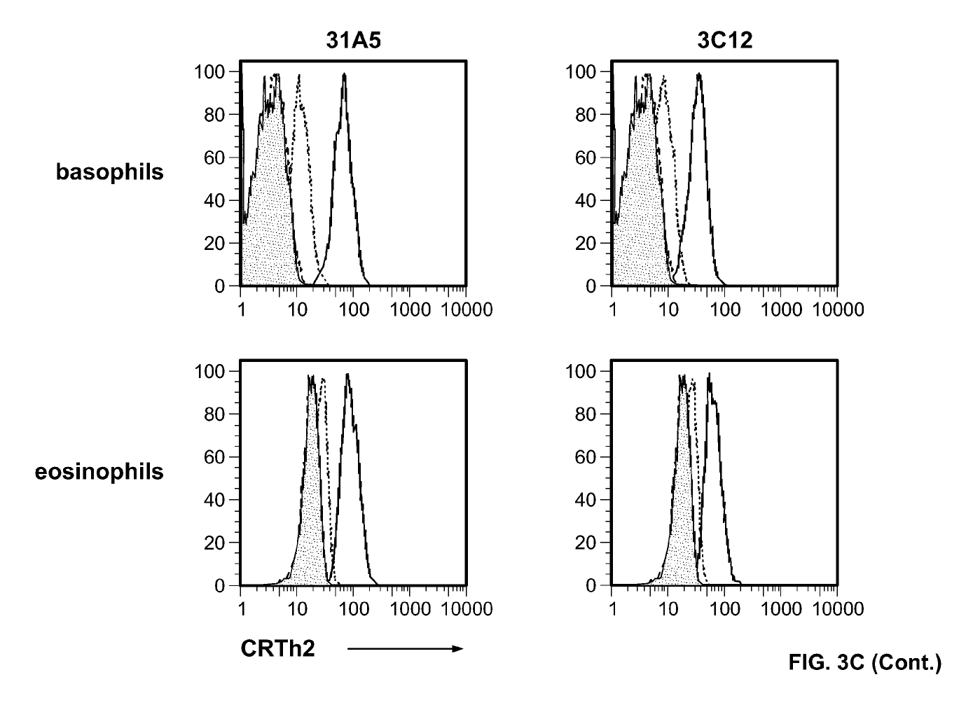

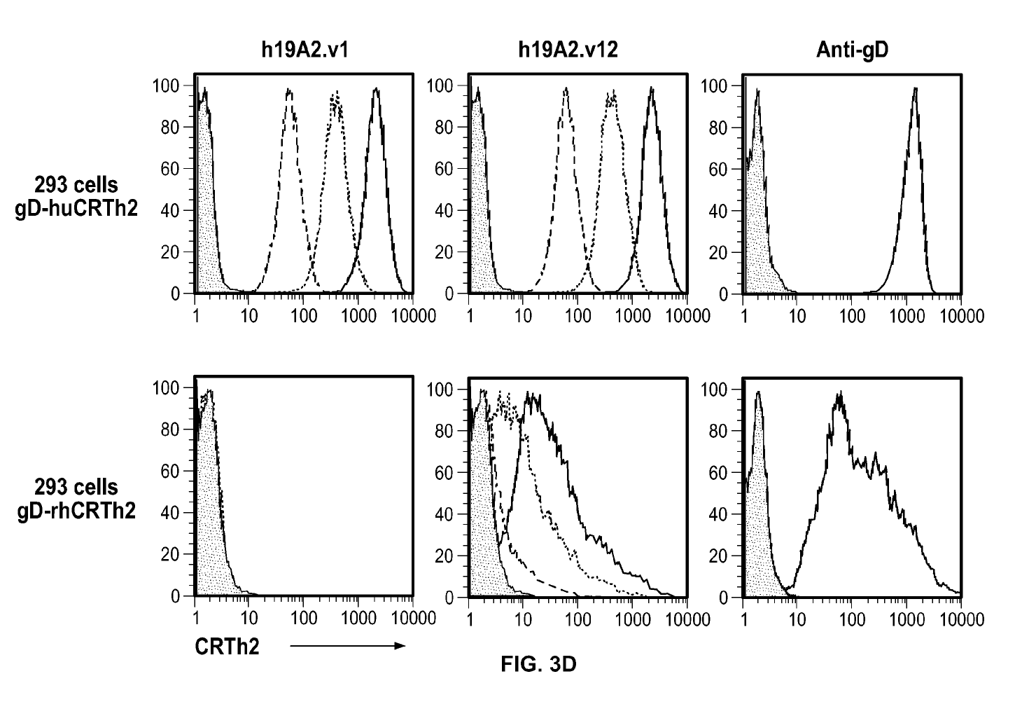

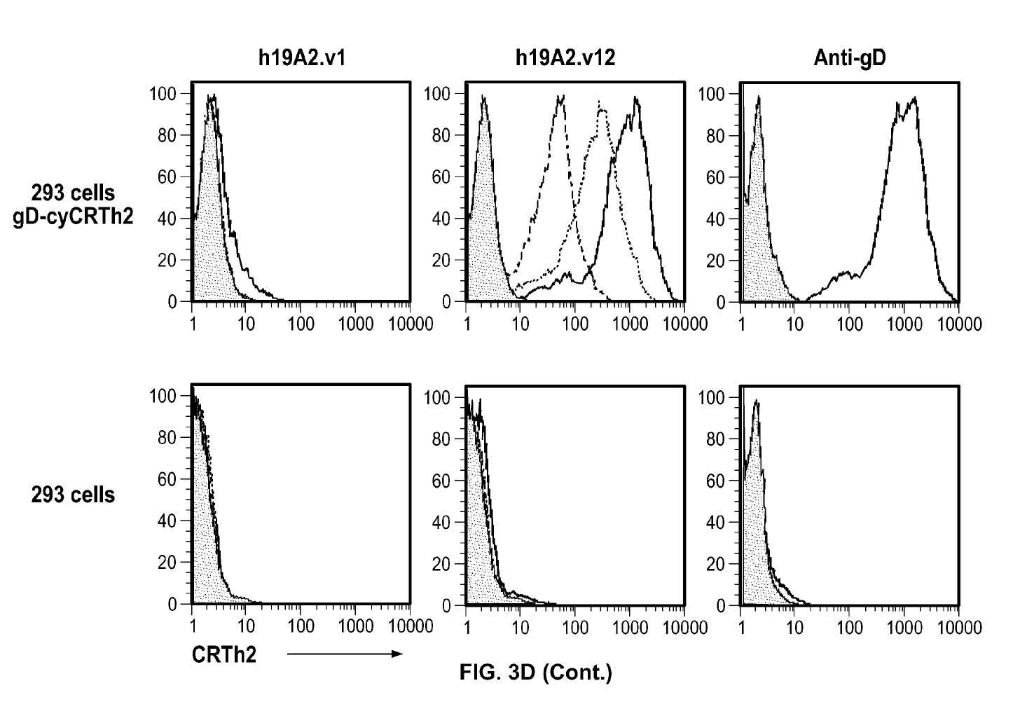

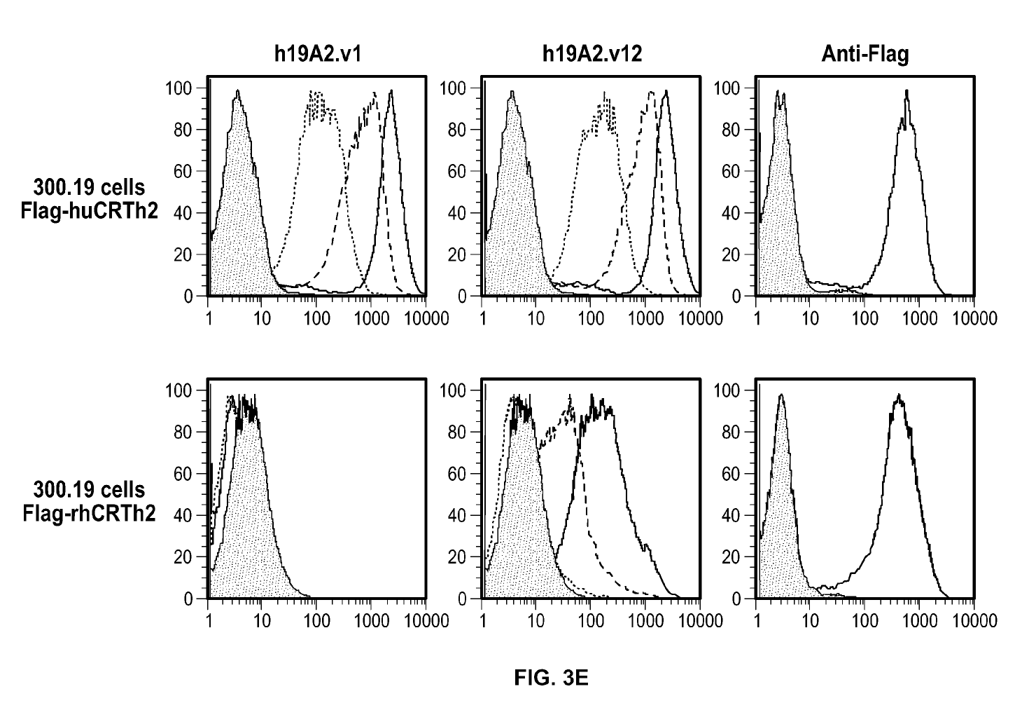

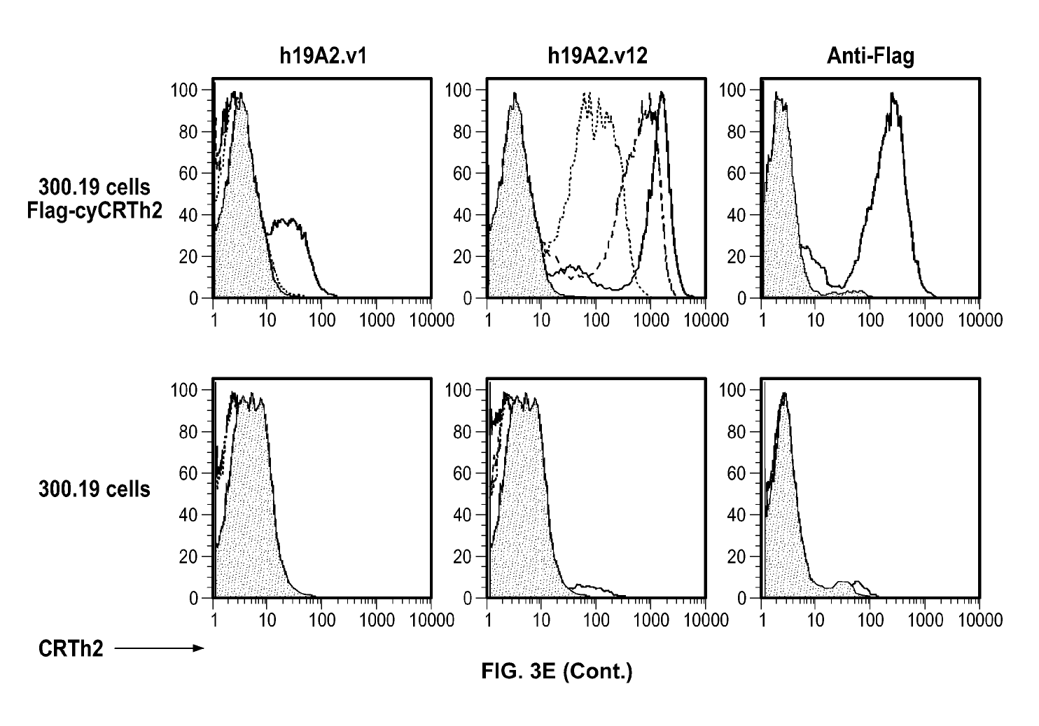

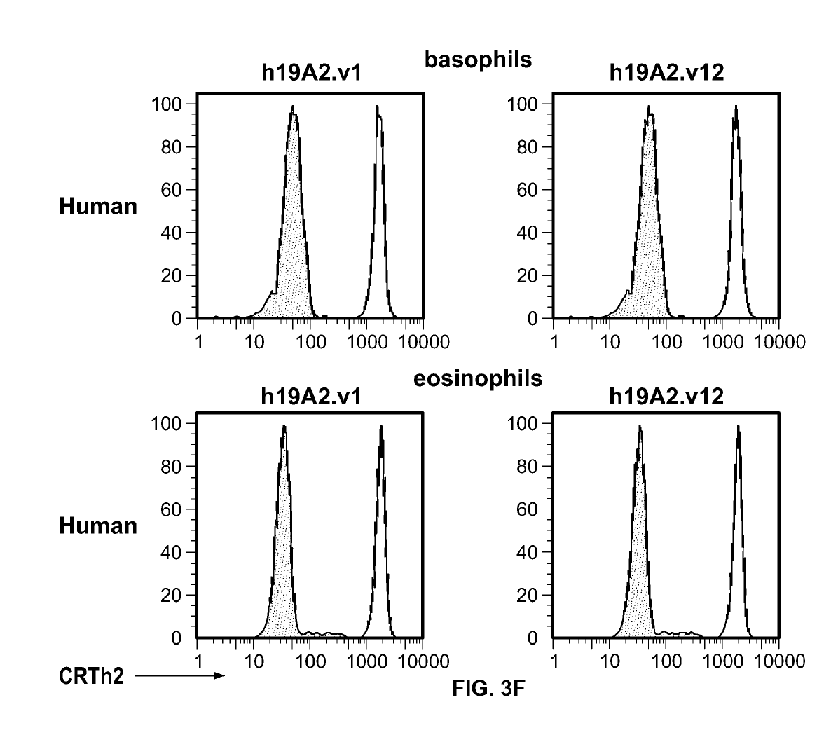

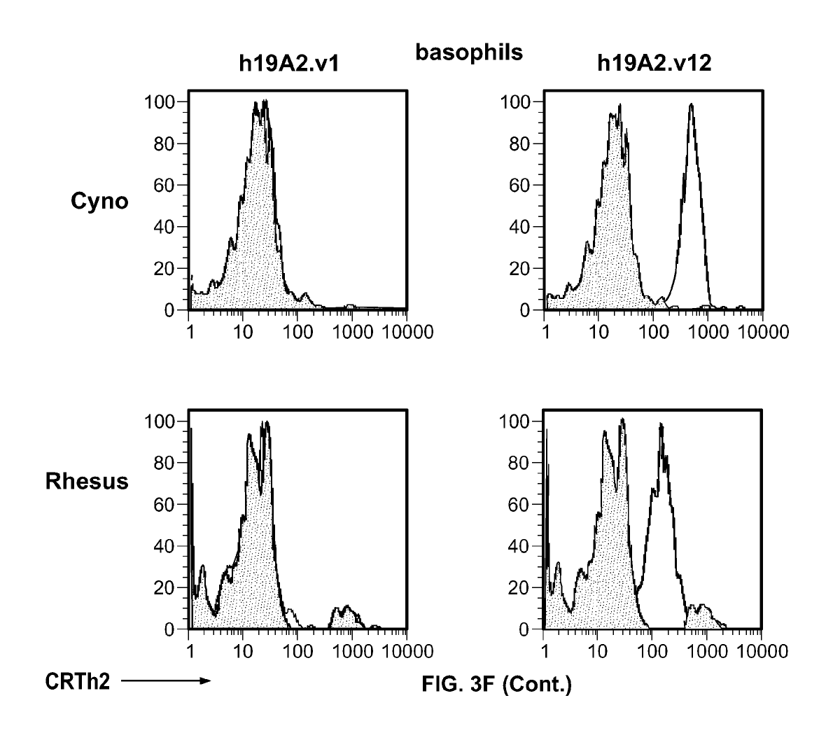

Anti-CRTh2 antibodies generated as described herein bound in a dose dependent manner to 293 cells or 300.19 cells overexpressing human CRTh2 but not to 293 or 300.19 wild-type cells (FIGS. 3A and 3B). Anti-human CRTh2 Abs were also tested for cross-reactivity with cynomolgus (cyno) or rhesus monkey CRTh2 overexpressed in 293 or 300.19 cells. None of the Abs showed reactivity with cyno or rhesus CRTh2 except clone 19A2, which showed a minor cross-reactivity to cyno CRTh2 expressed on 293 cells. Anti-human CRTh2 antibodies also reacted with primary human basophils and eosinophils from human whole blood in a dose dependent manner Candidate anti-human CRTh2 antibodies were selected based on their ability to bind human CRTh2 overexpressed on the surface of 293 cells or 300.19 cells, as well as their relative reactivity with primary basophils and eosinophils from human peripheral blood mononuclear cells (PBMC) (FIG. 3). All of the additional antibodies generated from the immunization described above bound to human CRTh2, but did not cross-react with rhesus or cyno CRTh2 (data not shown). Humanized clones h19A2.v1 and clone h19A2.v12 were also tested for reactivity with CRTh2 expressed on 293 cells or 300.19 cells as well as with CRTh2 on primary blood basophils and eosinophils. Similar to 19A2, humanized h19A2.v1 reacted with human CRTh2 expressed on 293 cells (FIG. 3D), 300.19 cells (FIG. 3E) and CRTh2 on primary blood basophils and eosinophils (FIG. 3F) with a minor cross-reactivity to cyno CRTh2 over-expressed on 293 or 300.19 cells. Humanized h19A2.v1 did not react with rhesus CRTh2 on overexpressing 293 or 300.19 cell lines and primary rhesus blood basophils. In contrast, humanized and engineered antibody h19A2.v12 reacted in a dose-dependent manner with human, cynomolgus and rhesus CRTh2 expressed on 293 cells (FIG. 3D) or 300.19 cells (FIG. 3E) as well as with human, cyno and rhesus CRTh2 on primary blood basophils (FIG. 3F). Furthermore, antibody h19A2.v12 also detected CRTh2 on primary human blood eosinophils.

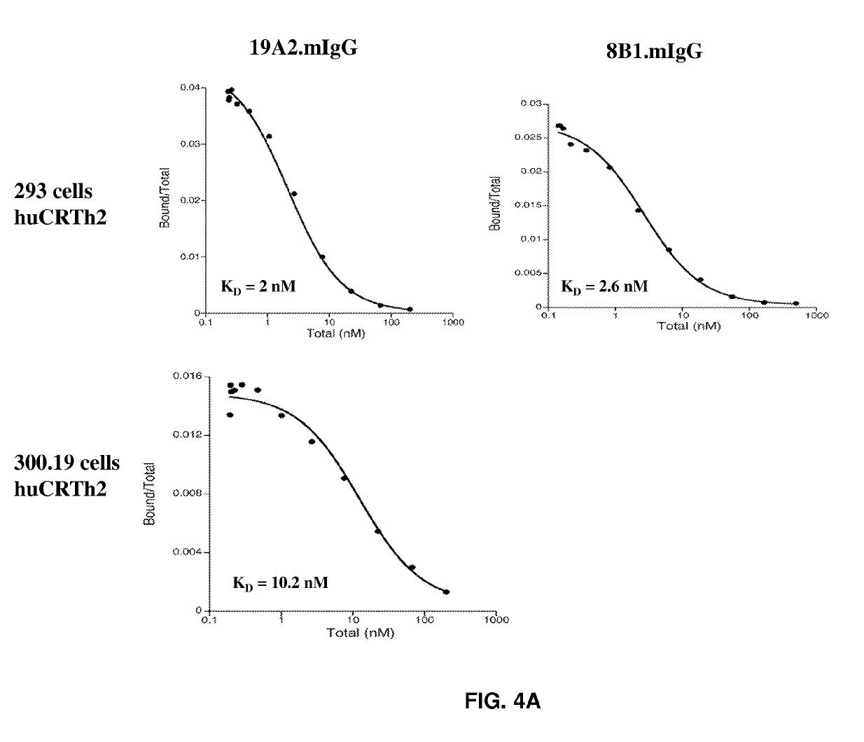

Radiolabeled ligand analysis with homologous competition was performed to assess the dissociation constant (K_{D}) of anti-CRTh2 antibodies to surface expressed human CRTh2 on 293 cells and 300.19 cells. The K_{D} values for mouse anti-human CRTh2 clones 19A2 and 8B1 (whole IgG) to 293 cell expressing human CRTh2 were 2 nM and 2.6 nM, respectively. The K_{D} value of antibody 19A2 to 300.19 cells was 10.2 nM (FIG. 4A). To obtain a direct measurement of the K_{D} value of humanized antibodies h19A2.v12 and h19A2.v60, Fab fragments of these antibodies were generated and subjected to radioligand cell binding assays (FIG. 4B). The K_{D} values for h19A2.v12 and h19A2.v60 (Fab fragment of IgG) to 293 cell expressing human CRTh2 were 51 nM and 56 nM, respectively. The K_{D} values for h19A2.v12 and h19A2.v60 (Fab fragment of IgG) to 293 cells expressing cynomolgus monkey CRTh2 were 152 nM and 39 nM, respectively. Based on these measurements, the relative binding affinity for human versus cyno CRTh2 is within 3-fold for h19A2.12 and appears equipotent for h19A2.v60 (FIG. 4B).

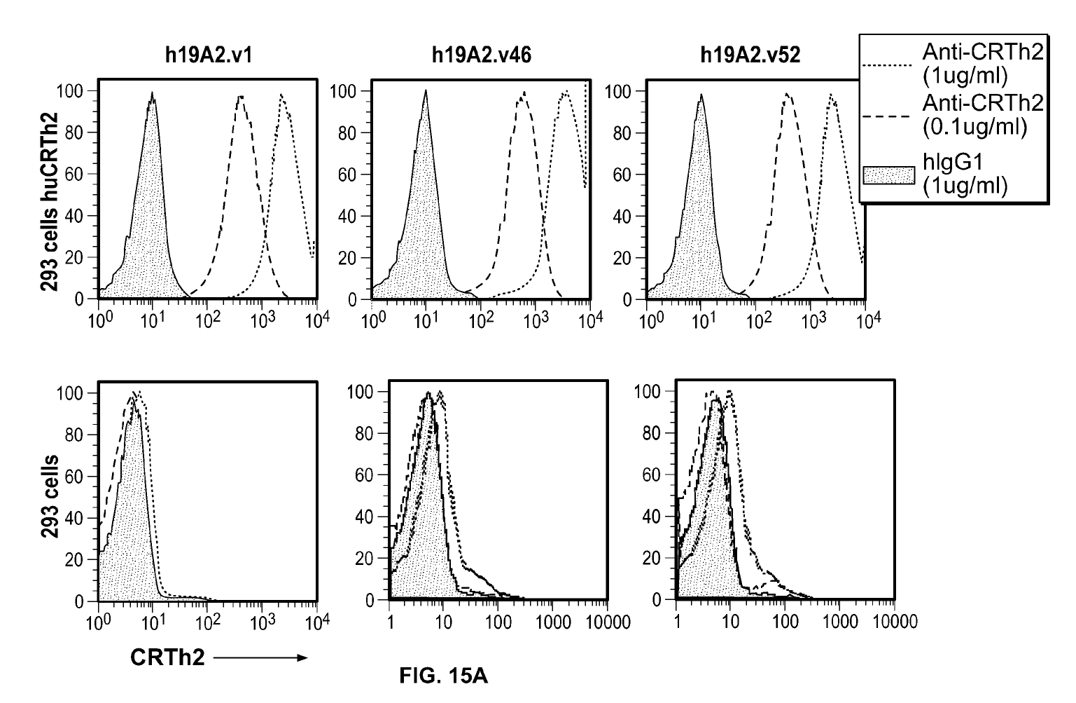

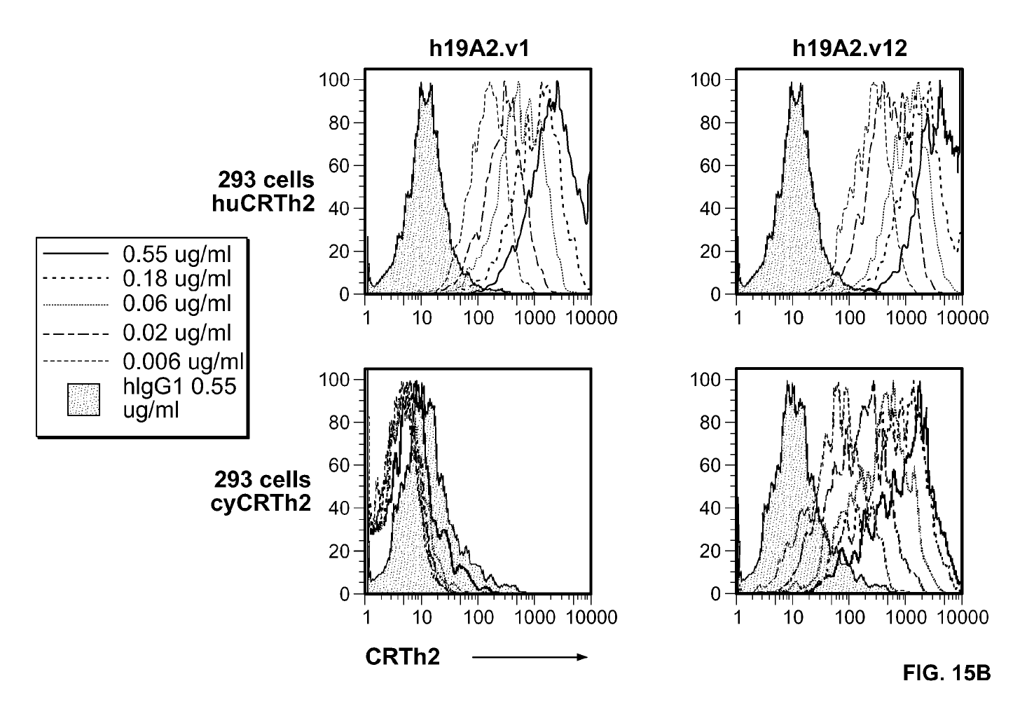

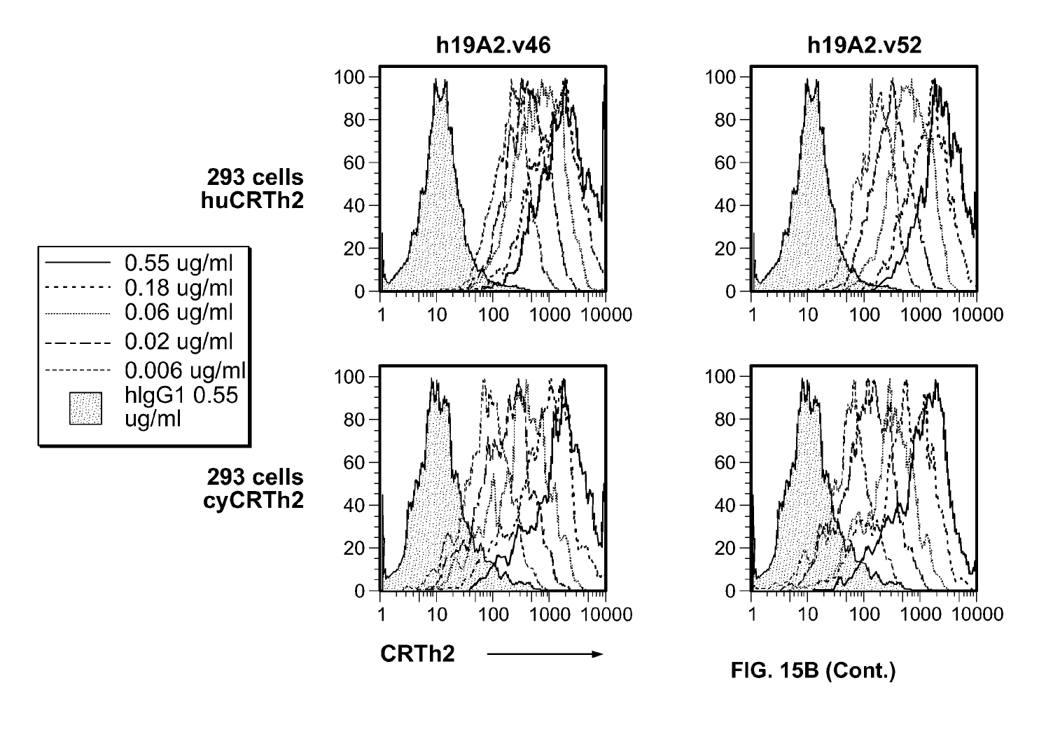

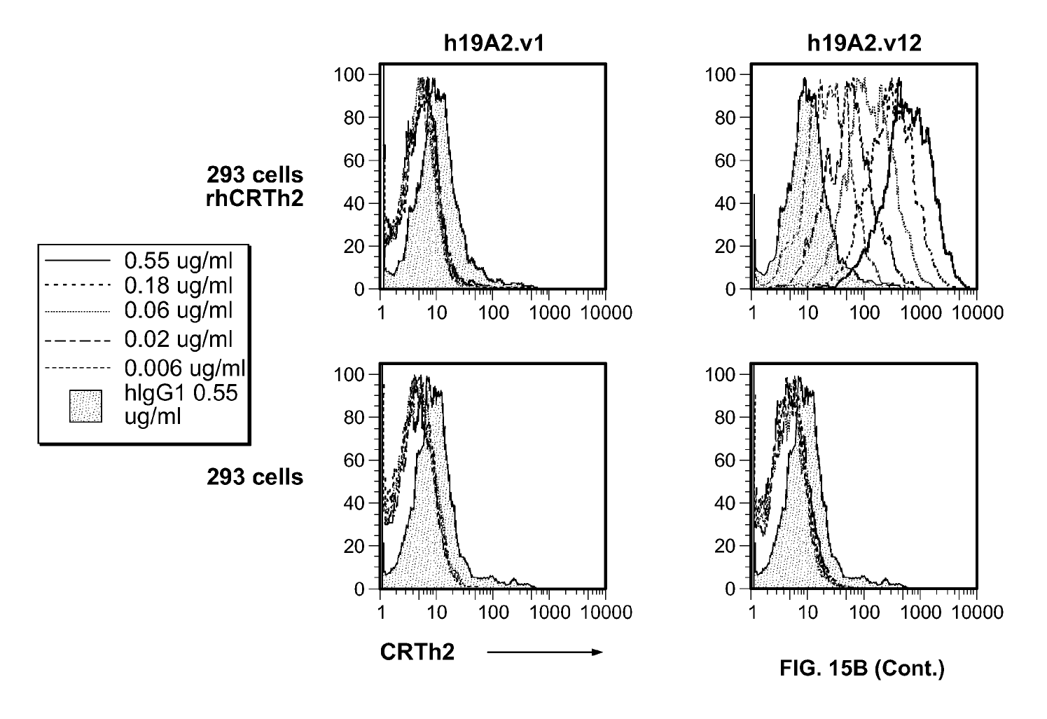

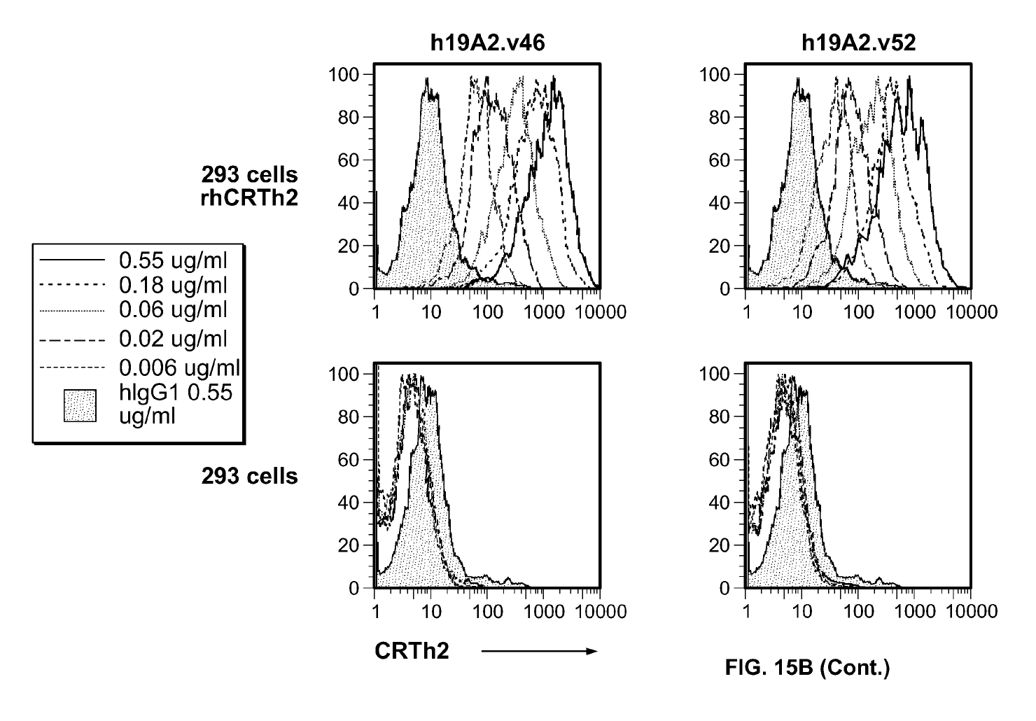

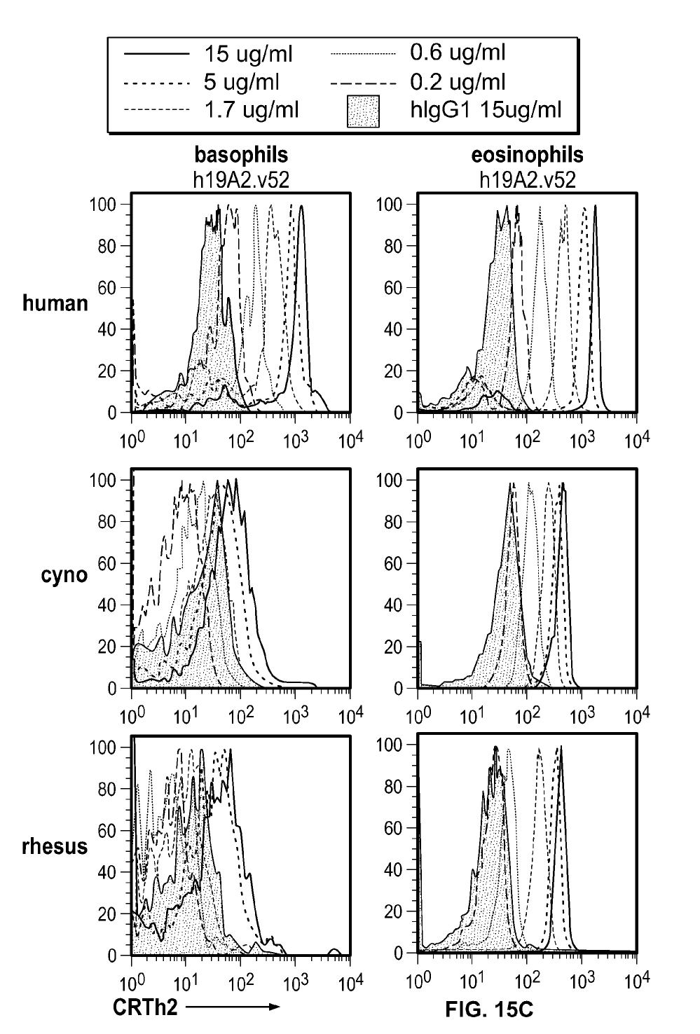

To obtain a direct measurement of the K_{D} value of humanized antibodies h19A2.v52 and h19A2.v46, Fab fragments of these antibodies were generated and subjected to radioligand cell binding assays (FIG. 15). The K_{D} values for h19A2.v52 and h19A2.v46 (Fab fragment of IgG) to 293 cell expressing human CRTh2 were 13.7 nM and 6.4 nM, respectively. The K_{D} values for h19A2.v52 and h19A2.v46 (Fab fragment of IgG) to 293 cells expressing cynomolgus monkey CRTh2 were 21.3 nM and 8.6 nM, respectively.

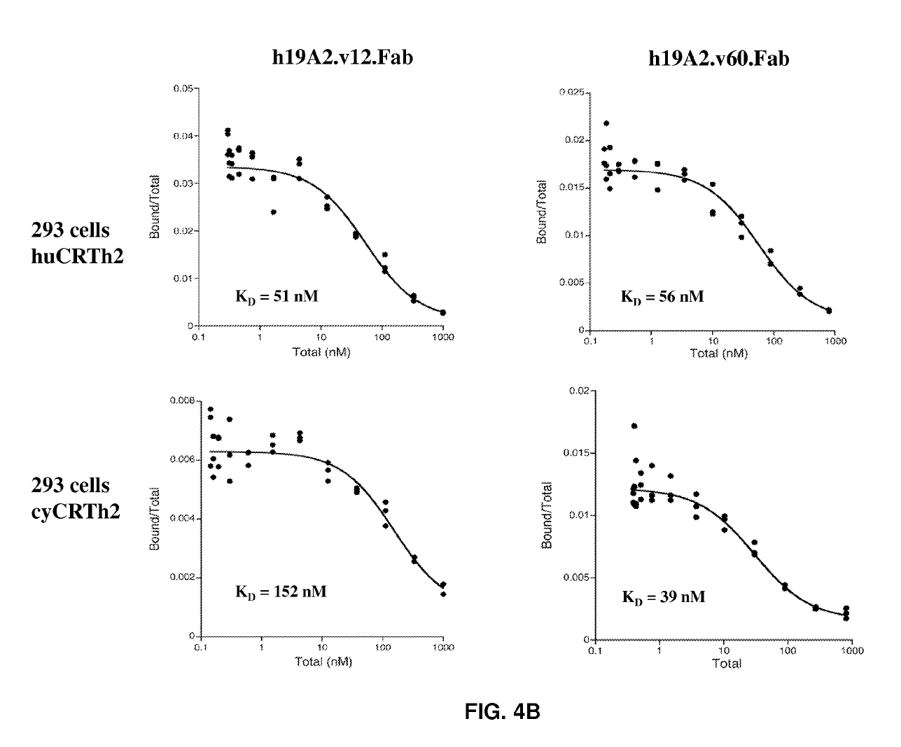

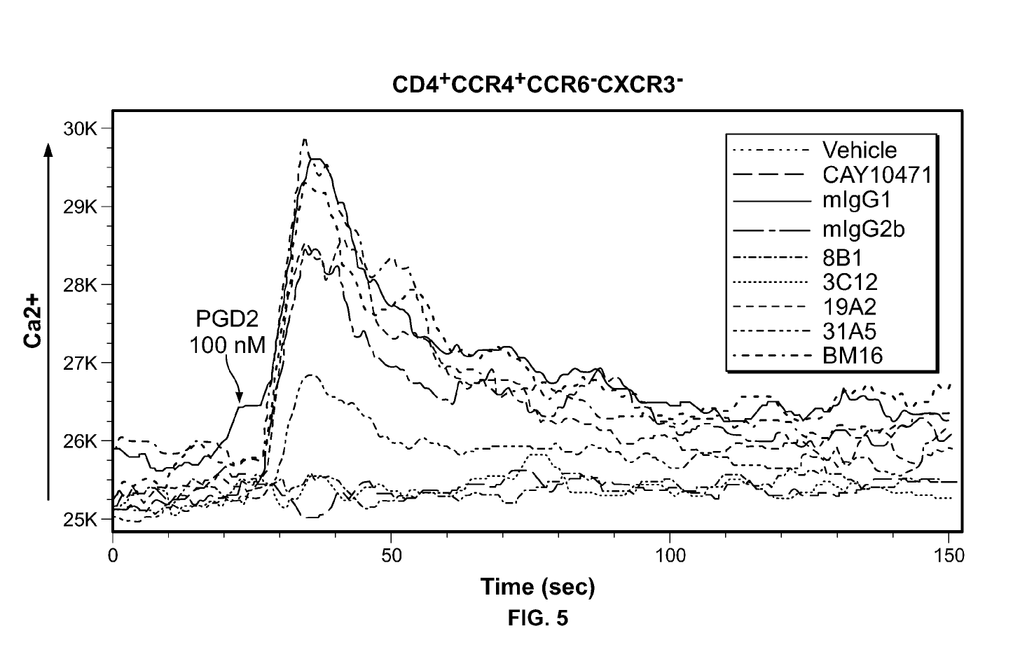

To assess the blocking function of the anti-CRTh2 antibodies, calcium mobilization of in vitro polarized Th2 cells to the ligand prostaglandin (PGD2) was examined in the presence of anti-CRTh2 or isotype control antibodies. Calcium flux to PGD2 was completely prevented by pre-incubation of cells with 8B1 and 3C12, while 31A5 showed a partial effect. Incubation with anti-CRTh2 19A2 antibody did not significantly affect CA2+ flux (FIG. 5), indicating that 19A2 is a non-blocking antibody to CRTh2, in contrast to 8B1 and 3C12 that can block the function of CRTh2.

Generation of a Transgenic Mouse Model of Human CRTh2

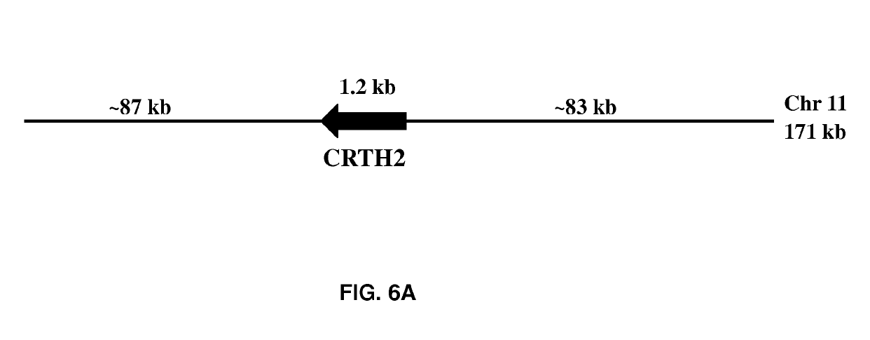

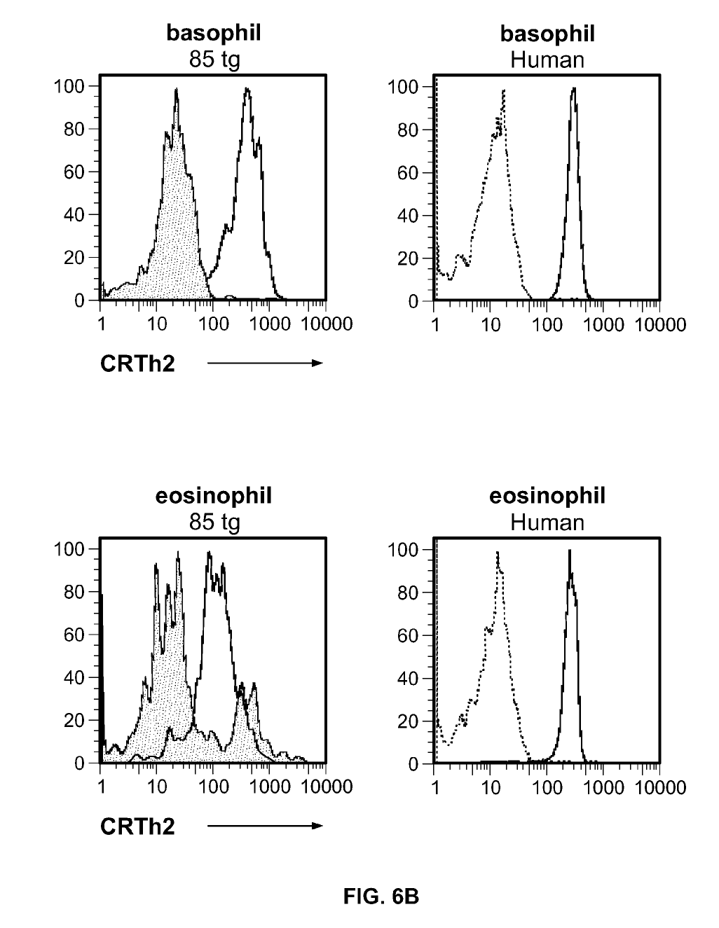

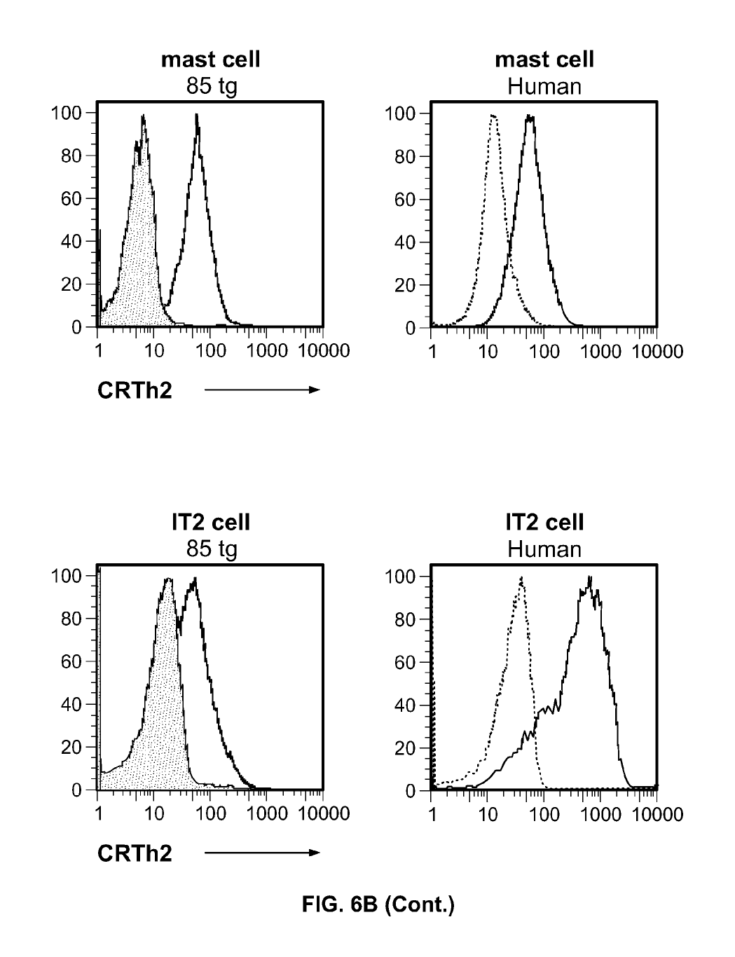

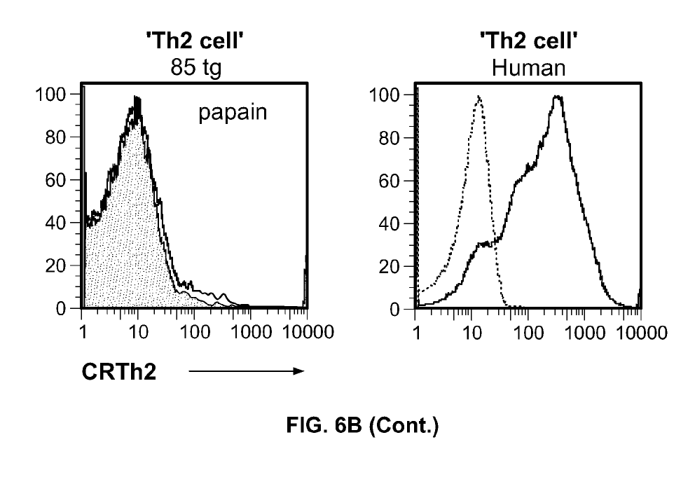

In order to characterize the depleting capacities of anti-CRTh2 antibodies in vivo, a transgenic mouse model (human CRTh2.Bac.Tg mice) was generated by introducing the human CRTh2 gene on a BAC vector into C57BL/6 fertilized oocytes (FIG. 6A). While human CRTh2 expression on blood basophils and eosinophils was confirmed in seven founders, expression of hCRTh2 on mouse Th2 cells in the hCRTh2.Bac.tg lines could not be detected. Three representative founder lines were subjected to more detailed analyses (data not shown). Founder line 85 of the human CRTh2.Bac.Tg mice demonstrated similar expression level of human CRTh2 on mouse blood basophils and eosinophils, as well as peritoneal mast cells when compared to primary human blood basophils and eosinophils, as well as bone-marrow derived human mast cells (FIG. 6B), respectively. Therefore, founder 85 hCRTh2.Bac.Tg mice were used in all the subsequent in vivo depletion studies. Furthermore, founder line 85, expressed human CRTh2 on mouse innate T helper type (IT) 2 cells (FIG. 6B) albeit expression levels appeared lower when compared to expression on human IT2 cells from PBMC.

### Anti-CRTh2 Antibodies Depleted Blood Basophils and Eosinophils in Human CRTh2.Bac.Tg Mice

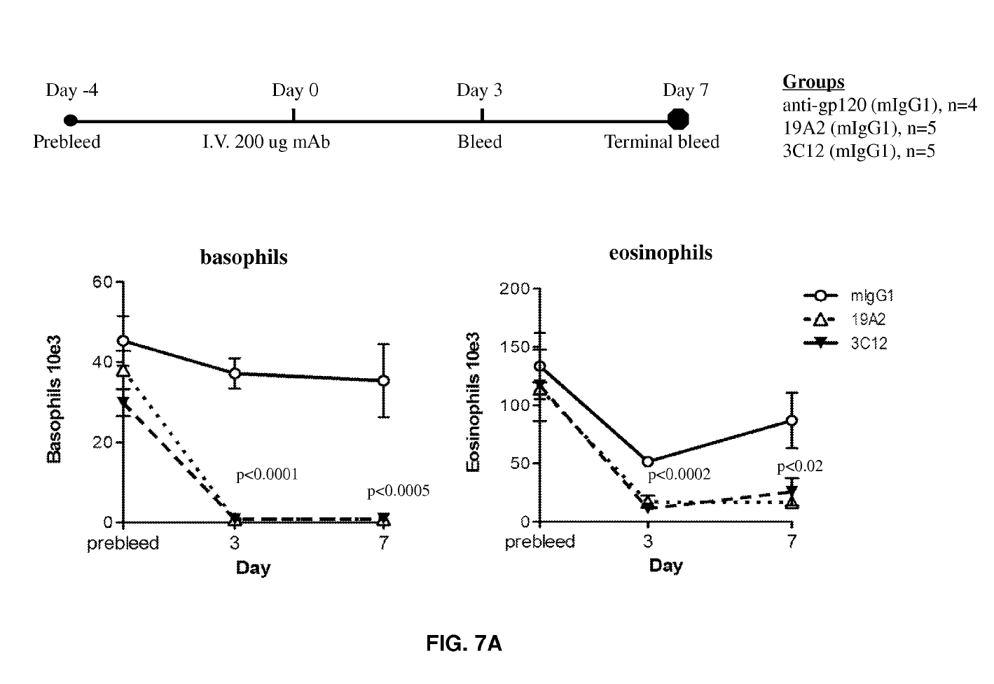

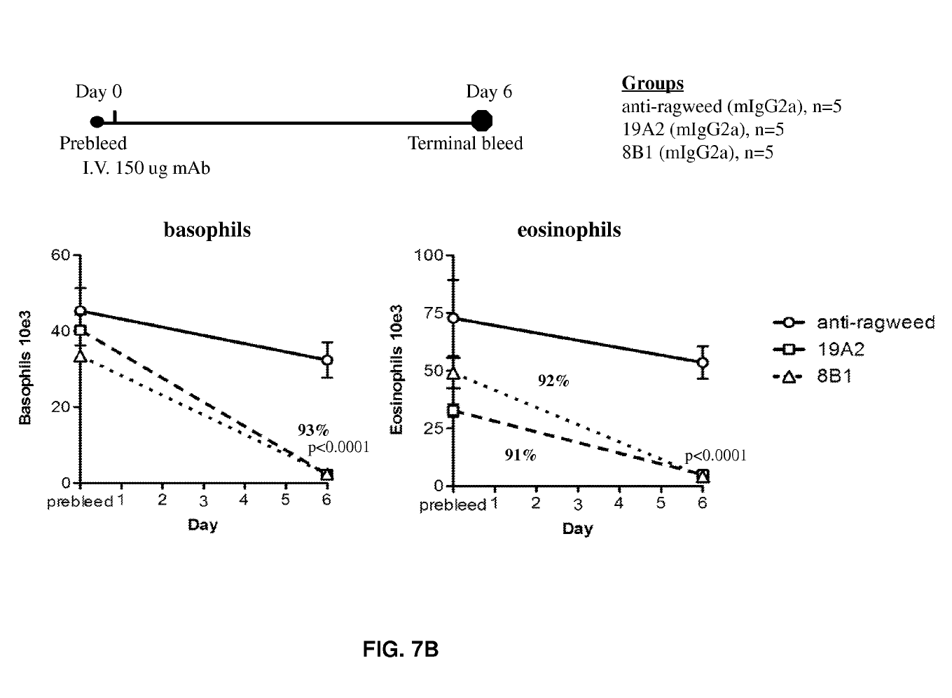

To test whether anti-CRTh2 antibodies can deplete CRTh2+ basophils and eosinophils in vivo, one dose of either 19A2 or 3C12 antibody was administered to CRTh2.Bac.Tg mice as indicated (FIG. 7A). A single dose of 19A2 or 3C12 completely depleted basophils and eosinophils in peripheral blood in human CRTh2.Bac.Tg mice on day 3 after treatment as determined by flow cytometry (FIG. 7A). Significant depletion of basophils and eosinophils was still observed on day 7 after treatment. 8B1 and 19A2 antibodies also depleted basophils and eosinophils from blood after a single dose of antibody as assessed on day 6 after treatment (FIG. 7B).

Anti-CRTh2 Antibodies Deplete Eosinophils and Basophils in Lung in TNP-OVA Induced Chronic Asthma Model in Human CRTh2.Bac.Tg Mice

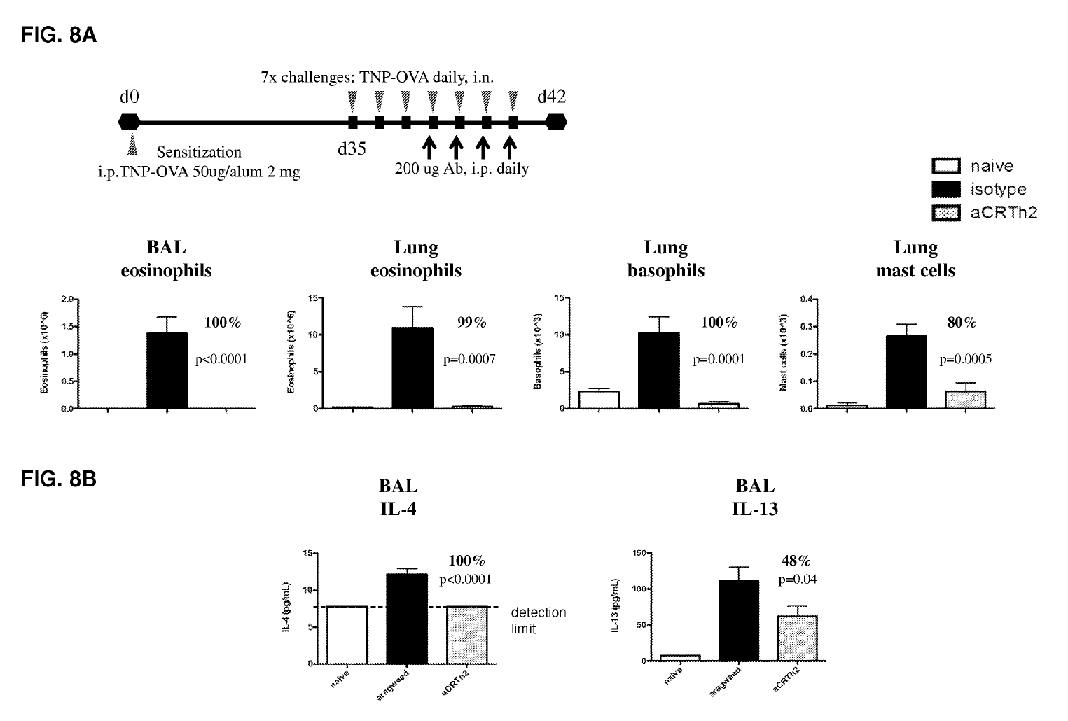

To assess whether anti-CRTh2 antibodies can deplete CRTh2+ cells within tissues, the effect of anti-CRTh2 antibody treatment was examined in a TNP-OVA induced chronic asthma model. Four doses of antibody 19A2 in a therapeutic regimen of i.p. 200 ug/mouse, depleted lung eosinophils and basophils completely, and also depleted lung mast cells by 80% (FIG. 8). In addition, eosinophils in the bronchial alveolar lavage fluid (BALF) were 100% depleted. Furthermore, the Th2 cytokines IL-4 and IL-13 in the bronchial alveolar lavage fluid (BALF) were reduced by 100% and 48%, respectively (FIG. 8B).

Anti-CRTh2 Antibodies Deplete Th2 Cytokine Producing Cells in SCID Mice

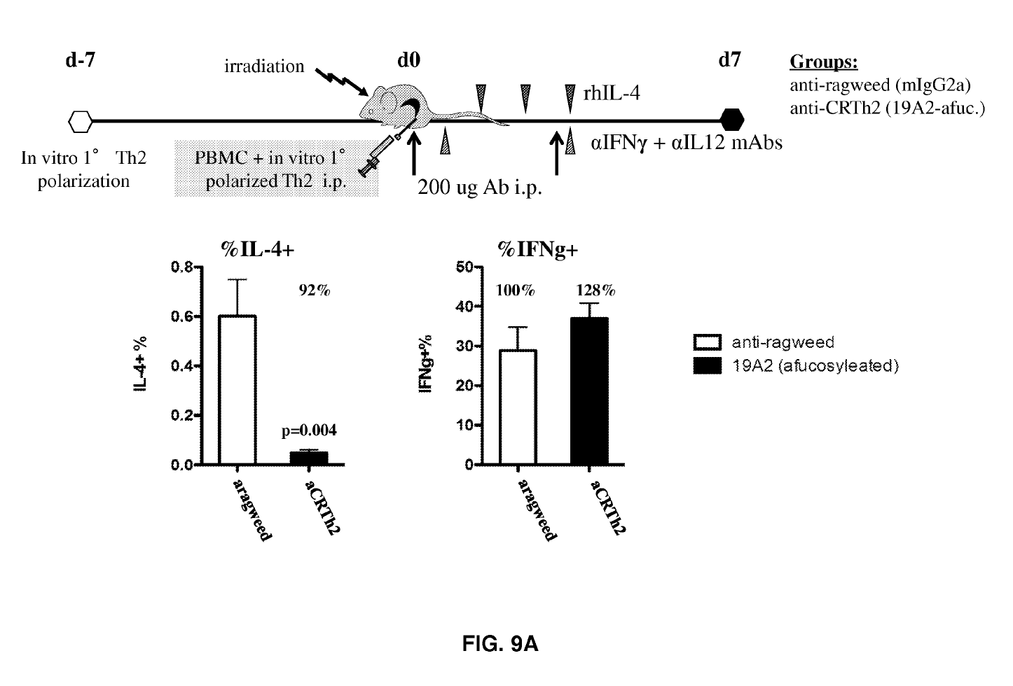

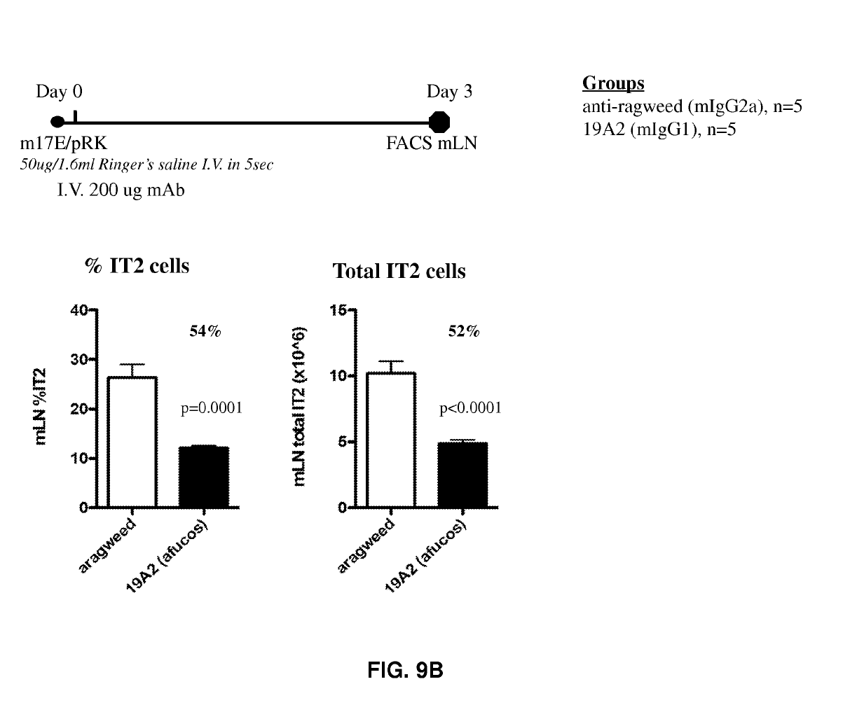

Since human CRTh2 expression is not detected on Th2 cells (CD4+CD44hi) in human CRTh2.Tg mice, Th2 cell depletion could not be assessed in human CRTh2.Bac.Tg mice. To evaluate whether anti-CRTh2 antibodies can deplete Th2 cytokine producing cells in vivo, in vitro polarized human Th2 cells were transferred into SCID mice, treated with anti-CRTh2 or isotype control Abs twice a week, and IL-4 producing cells were assessed after ex vivo stimulation with PMA and Ionomycin on day 7 after dosing start. Intracellular IL-4 staining indicated that 92% of IL-4 producing cells were depleted with 19A2 anti-CRTh2 antibody treatment while IFNg producing cells were not reduced (FIG. 9A).

Anti-CRTh2 Depletes Innate Type 2 Cells in Human CRTh2.Bac.Tg Mice

To assess the ability of anti-CRTh2 antibodies to deplete innate type 2 (IT2) cells (also termed innate lymphoid type 2 cells or ILC2 cells), IT2 cell numbers were increased by injection of an IL-17E containing plasmid into hCRTh2.Bac.Tg mice. Mice were treated with a single dose of anti-hCRTh2 or isotype control antibody i.v. and IT2 cell percentage and numbers were detected in mesenteric lymph nodes by flow cytometry on day 3 after treatment. Anti-hCRTh2 treatment significantly reduced by over 50% the percentage and number of mesenteric lymph node IT2 cells in hCRTh2.Bac.Tg mice.

### Example 2

### Antibody Humanization and Affinity Maturation

Expression of Biotinylated CRTh2 in Mammalian Cells

Human, cynomologous and rhesus CRTh2 cDNAs and human CRTh2 with the Q16E or R19H mutations were cloned into the mammalian expression vector pRK5 fused in frame in the 3′ end to a sequence encoding and linker and Avitag sequence (GSGGLNDIFEAQKIEWH). The BirA biotin ligase gene from Escherichia coli was also cloned into the mammalian expression vector pRK5. Plasmids encoding CRTh2 from each species were mixed with the BirA expression plasmid at a ratio of 9:1 and co-transfected into 293T cells using Lipofectamine2000 reagent (Invitrogen) in Dulbecco's Modified Eagle's medium containing 10% fetal bovine serum and supplemented with 10 μM biotin. Cells were harvested 24 hours post-transfection and the plasma membrane fractions containing biotinylated CRTh2 purified.

Purification of Plasma Membrane Fractions

Transfected cells (2.5×10⁸) from were washed twice in PBS (150 mM NaCl, 10 mM sodium phosphate, pH 7.4) containing protease inhibitor cocktail mix (Roche) and cell pellets were frozen at −80° C. Cells were thawed and resuspended in 4 ml of lysis buffer (1 mM EDTA, 50 mM HEPES buffer, pH7.4, containing protease inhibitor mix) and lysed in a Dounce homogenizer with 8 strokes with a tight-fitting pestle. After initial lysis, 4 ml of lysis buffer containing 500 mM sucrose were added and further homogenized with 8 strokes with a tight-fitting pestle. Cell debris were removed by centrifugation for 10 min at 770×g and membrane material in the supernatant pelleted by centrifugation at 17,000×g. The pelleted membranes were resuspended in 6 ml of lysis buffer containing 250 mM sucrose with 8 strokes of a loose-fitting pestle in a Dounce homogenizer. Large debris were removed by centrifugation at 770×g for 10 minutes. The supernatant was carefully laid on 4 ml of lysis buffer containing 1.12 M sucrose in a translucent SW40 centrifuge tube and spun in a SW40Ti rotor (Beckman) at 25,000 rpm for 1 hour at 4° C. The material at the interface between the high and low-concentration sucrose fractions was collected with a pipette, mixed with an equal volume of lysis buffer without sucrose and pelleted by centrifugation at 16,000×g at 4° C. for 10 min. The pelleted plasma membranes were resuspended in 1 ml of lysis buffer and stored at −80° C. All homogenization steps were performed on ice.

Expression and Purification of CRTh2 from Insect Cells

Protein expression: DNA encoding Ala3-Asp330 of Homo sapiens and Macaca fascicularis CRTh2 was cloned into a modified pAcGP67 baculovirus transfer vector (BD Biosciences) containing a C-terminal Avi-tag and His8-tag. Recombinant baculovirus was generated by cotransecting Sf9 cells with the pAcGP67 construct and linearized baculovirus DNA in ESF 921 media (Expression Systems) using the BaculoGold Expression System (BD Biosciences). Virus was generated through three rounds of amplification. Recombinant baculovirus expressing untagged Escherichia coli BirA (Met1-Lys321) was similarly generated. Forty mL of both viruses (CRTh2 and BirA) were used to co-infect 10 L of Tni.PRO cells at a density of 2×10⁶ cells/mL. Cells were further grown for 48 hr at 27° C. and removed from the media by centrifugation.

Protein purification: Harvested cell pellets were resuspended and lysed in 50 mM Tris pH 8, 200 mM NaCl (TBS) containing Complete EDTA-free Protease Inhibitor Cocktail (Roche) by three passages through a microfluidizer. After clarification of the lysate, membranes were harvested by centrifugation at 40K in a 45 Ti ultracentrifuge rotor (Beckman) for 2 hr at 4° C. Thirty grams of membrane pellet was resuspended in TBS (10 g/L) and solubilized with 1% (wt/vol) lauryl maltose neopentyl glycol (LMNG, Affymetrix) for 2 hr at 4° C. After clarification, samples were batch-bound on Ni-NTA resin (Qiagen) and washed with TBS containing 0.12% (wt/vol) digitonin (EMD Biosciences) containing 15 mM imidazole. Proteins were eluted with the same buffer containing 300 mM imidazole, and concentrated and diluted five times in TBS-digitonin (0.12%) buffer without imidazole using 100 MWKO spin concentrators at 4° C. (Vivaspin, GE Healthcare). Biotinylated-CRTh2 protein concentrations were estimated by comparison to protein standards; samples were aliquoted and snap frozen in liquid nitrogen.

ELISA with Solubilized CRTh2

Neutravidin (Pierce) was coated on 96-well Maxisorp ELISA plates (2 μg/ml in 10 mM carbonate buffer, pH 9.6, 100 μl per well) overnight at 4° C. and blocked with PBS containing 0.5% bovine serum albumin (blocking buffer). Plasma membranes containing CRTh2 or control membrane protein or purified CRTh2 were diluted in blocking buffer and lysed in 1% dodecylmatoside (DDM) for 15 minutes on ice and insoluble material removed by centrifugation at 16,000×g at 4° C. for 30 min Solubilized CRTh2 or control membrane protein were diluted in blocking buffer containing 0.2% DDM and added to neutravidin-coated plates. Protein was incubated for 10 minutes and plates were washed with PBS containing 0.05% DDM. Antibodies were serially diluted in blocking buffer containing 0.2% DDM and incubated with captured antigen for 1 hour at 4° C. Plates were washed as described above and anti-human or anti-mouse IgG conjugated to peroxidase diluted in blocking buffer containing 0.2% DDM was added to the plates. After 30 min incubation at 4° C. the plates were washed as described above and TMB substrate was added to the plates. The peroxidase reaction was stopped with an equal volume of 1 M phosphoric acid and optical absorbance was read at 450 nm. The amount of CRTh2 protein used was sufficient to attain saturation of wells as determined by ELISA using an anti-CRTh2 Mab binding recombinant human, cynomolgus and rhesus CRTh2.

Antibody Humanization

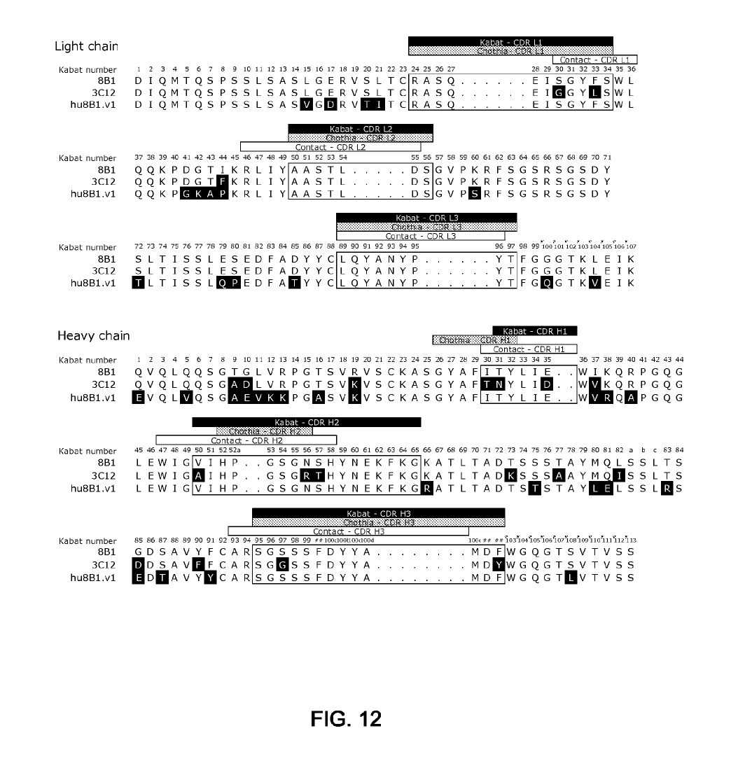

The CDR sequences of Mab 19A2 (FIG. 10), were grafted on a consensus kappa 1 (Consensus K1) and consensus VH3 (Consensus H3) framework (Dennis, M. S. (2010). CDR repair: A novel approach to antibody humanization. In Current Trends in Monoclonal Antibody Development and Manufacturing, S. J. Shire, W. Gombotz, K. Bechtold-Peters and J. Andya, eds. (Springer, New York), pp. 9-28) by oligonucleotide-directed site mutagenesis. Framework residues in position 71 of the light chain (Kabat numbering system) and 49 of the heavy chain that were present in the parental murine 19A2 antibody were also incorporated into the framework positions of humanized antibody hu19A2.v1 (FIGS. 11A and 11B). The CDR sequences of Mab 8B1 (FIG. 12) were grafted on the Consensus K1 and consensus VH1 (Consensus H1) frameworks by oligonucleotide-directed site mutagenesis. Framework residues in positions 46, 66, 69 and 71 of the light chain and 37, 67, 69, 71 and 91 of the heavy chain that were present in the parental murine 8B1 antibody were also incorporated into the framework positions of humanized antibody hu8B1.v1 (FIG. 12). All antibodies were cloned in pRK5 vector. Humanized antibodies were expressed as human IgG1 in 293T cells and purified by affinity chromatography on protein A-sepharose. Binding of Mabs was determined by ELISA with human, cynomolgus and rhesus CRTh2.

Affinity Maturation

The heavy and light chain variable regions of hu19A2.v1 were cloned in a phage display vector that displays Fab fragments fused to the p3 protein of bacteriophage M13. Two sets of “stop” template vectors were prepared in which all 3 CDRs of the light or heavy chains were removed and replaced with sequences encoding stop codons. Libraries with randomized heavy or light chain CDRs were created by oligonucleotide-directed site mutagenesis. Oligonucleotides for each CDR were synthesized in which each oligonucleotide had one codon randomized as a NNK (N=A, T, C or G; K=T or G). Kabat positions 27 to 34, 50 to 56 and 89 to 97 of the light chain were randomized with a set of 24 oligonucleotides and Kabat positions 26 to 35, 49 to 58 and 95 to 100a with a set of 28 oligonucleotides. Randomized libraries were electroporated into E. coli XL1-Blue cells (Agilent technologies), infected with a mutant helper phage KO7+ (Lamboy et al., ChemBioChem 9: 2846-2852 (2008)) at a multiplicity of infection of 10 particle-forming units per cell, allowed to recover and grown overnight in 2YT broth containing 50 μg/ml Carbenicillin and 100 μg/ml Kanamycin at 37° C. Cells were removed by centrifugation and phage displaying Fab in supernatants was concentrated and purified by PEG precipitation (ref). Phage were submitted to four rounds of selection. In each round, phage in blocking buffer containing 0.2% DDM was incubated for 1 hour at 4° C. with DDM-solubilized human CRTh2 with the wild-type sequence or with the Q16E or R19H mutations bound to neutravidin-coated ELISA plates. Plates were washed with PBS containing 0.05% DDM and phage was eluted with 100 μl of 0.1 N HCl for 10 minutes. Phage was collected and the pH was neutralized by adding 1/8 volume of 1 M Tris base. Phage were used to infect E. coli XL1-Blue and propagated as described above. Phage from the fourth round were used to infect E. coli XL1-Blue and plated on LB containing 50 μg/ml Carbenicillin to obtain isolated clones. Clones were sequenced by the dyedeoxy chain terminator method and mutations in each position tabulated. Favored mutations were introduced into the humanized hu19A2.v1 human IgG1 clones and IgG was expressed in human 293T cells and purified by affinity-chromatography. Binding of IgG to human, rhesus and cyno CRTh2 was tested by ELISA. A second generation library was created based on hu19A2.v12 including the light chain mutation S31W and heavy chain mutation Y58D. This second generation library was selected as described above except that purified human and cynomolgus CRTh2 antigen expressed in Sf9 cells in and 0.12% digitonin instead of DDM was used in selections. In addition, position 31 of the heavy chain was randomized with two oligonucleotides with the degenerate codons NHK and VNK in that position that, combined, encode for all amino acids except tryptophan and cysteine. The Tryptophan in position 31 of the light chain was changed to Tyrosine in humanized 19A2.v58, 19A2.v60 and 19A2.v52. 19A2.v46 is identical in sequence to 19A2.52 except that it contains Tryptophan at position 31 rather than Tyrosine. The aspartic acid in position 56 was changed to glutamic acid in 19A2.v60 and other clones to remove an isomerization site.

Generation and In Vitro Characterization of Mouse and Humanized Anti-Human CRTh2 Antibodies

Humanized clones h19A2.v1, h19A2.v46, and h19A2.v52 were tested for reactivity with human CRTh2 expressed on 293 cells. Similar to 19A2, humanized anti-CRTh2 antibodies reacted with human CRTh2 expressed on 293 cells in a dose dependent manner (FIG. 15A). In addition, humanized affinity matured clones h19A2.v12, h19A2.v46, and h19A2.v52 also showed dose-dependent reactivity with cyno and rhesus CRTh2 expressed on 293 cells while humanized antibody h19A2.v1 showed no reactivity with cyno and rhesus CRTh2 expressed on 293 cells (FIG. 15B). Furthermore, humanized and affinity matured antibodies h19A2.v52 reacted with primary human, cyno and rhesus basophils and eosinophils from whole blood in a dose-dependent fashion (FIG. 15C).

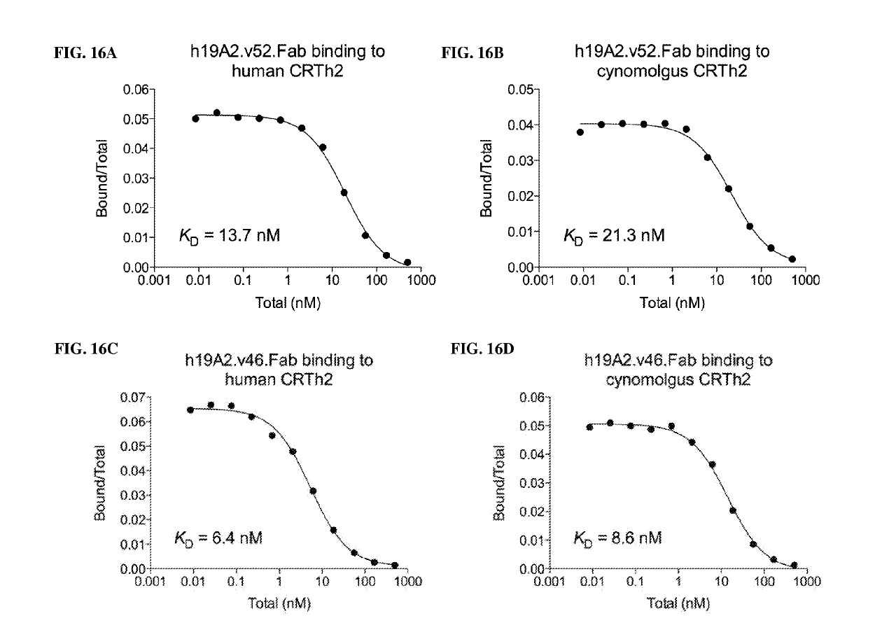

Radiolabeled ligand analysis with homologous competition was performed to assess the dissociation constant (KD) of anti-CRTh2 antibodies to surface expressed human and cynomolgus monkey CRTh2 on 293 cells. To obtain a direct measurement of the KD value of humanized antibodies h19A2.v52 and h19A2.v46, Fab fragments of these antibodies were generated (FIG. 16). The KD values for humanized anti-CRTh2 clones 19A2.v52 and 19A2.v46 (Fab fragment of IgG) to 293 cells expressing human CRTh2 were 13.7 nM and 6.4 nM, respectively. The KD values for h19A2.v52 and h19A2.v46 (Fab fragment of IgG) to 293 cell expressing cynomolgus monkey CRTh2 were 21.3 nM and 8.6 nM, respectively. Based on these measurements, the relative binding affinity for human versus cynomolgus CRTh2 is within 2-fold for h19A2.52 (FIGS. 16A and B) and appears close to equipotent for h19A2.v46 (FIGS. 16C and D).

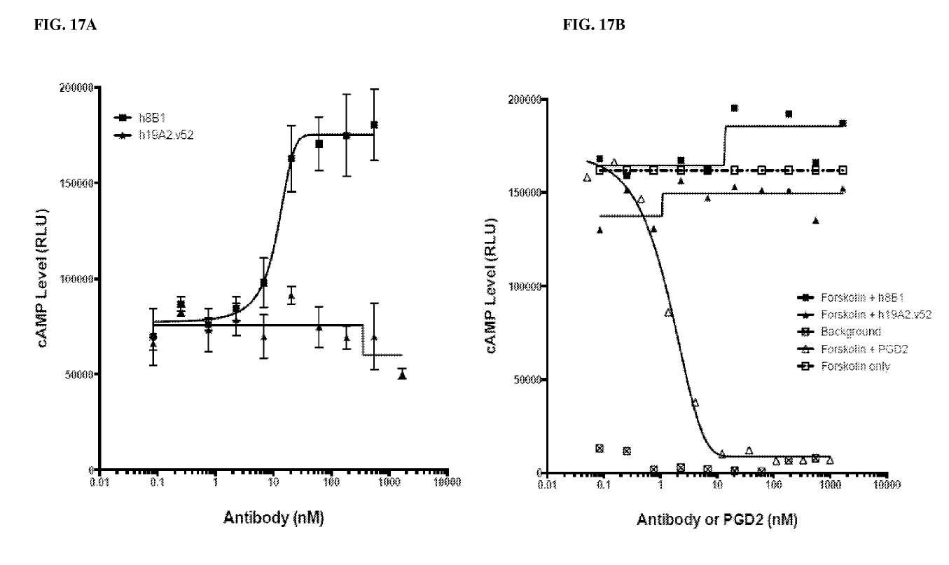

To assess the blocking function of the humanized and affinity matured anti-CRTh2 antibodies, the effect of anti-CRTh2 antibodies on PGD2-mediated inhibition of forskolin-induced cAMP levels in 293 cells expressing human CRTh2 was tested. Treatment of forskolin plus PGD2 stimulated hCRTh2 expressing 293 cells with the 8B1 antibody increased cAMP levels in a dose-dependent manner (FIG. 17 A). Thus, the 8B1 antibody blocked the PGD2-mediated decrease of forskolin-induced cAMP levels in a dose-dependent manner, indicating that 8B1 has PGD2 function blocking capacity similar to its ability to inhibit calcium flux in Th2 cells in response to PGD2. In comparison, h19A2.v52 did not show PGD2 function blocking capacity in the forskolin-induced cAMP human CRTh2 293 cell assay. It was also tested whether anti-CRTh2 antibodies had a direct effect on forskolin-induced cAMP levels in the absence of PGD2. As shown in FIG. 17B various concentrations of anti-CRTh2 antibodies showed no effect on forskolin-induced cAMP levels in human CRTh2 293 cells indicating that these antibodies do not exhibit agonistic activity in this assay. In comparison PGD2 reduced forskolin-induced cAMP levels.

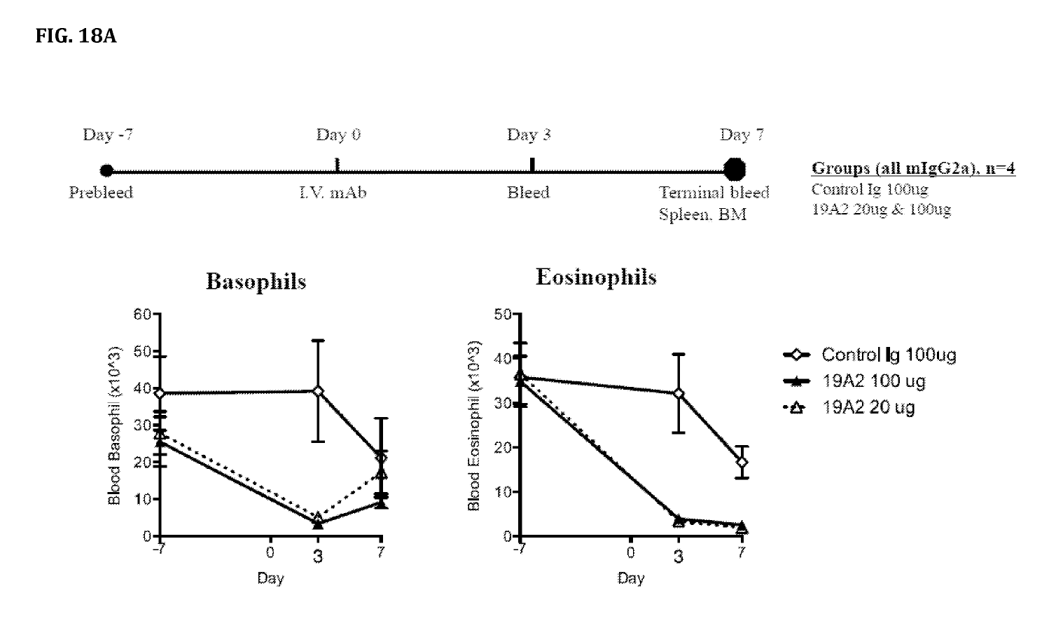

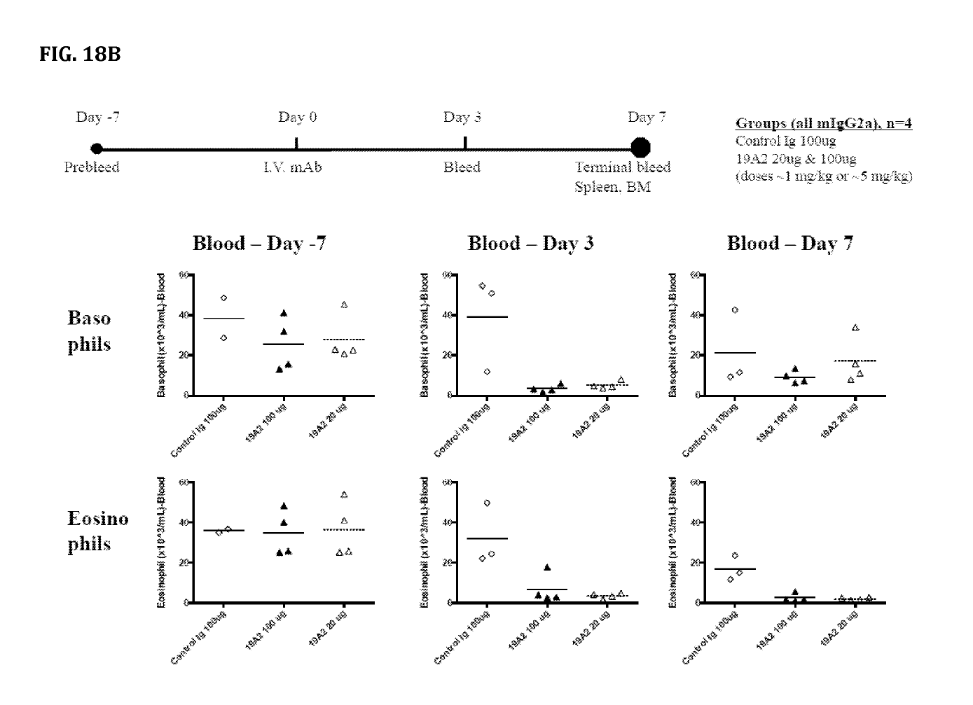

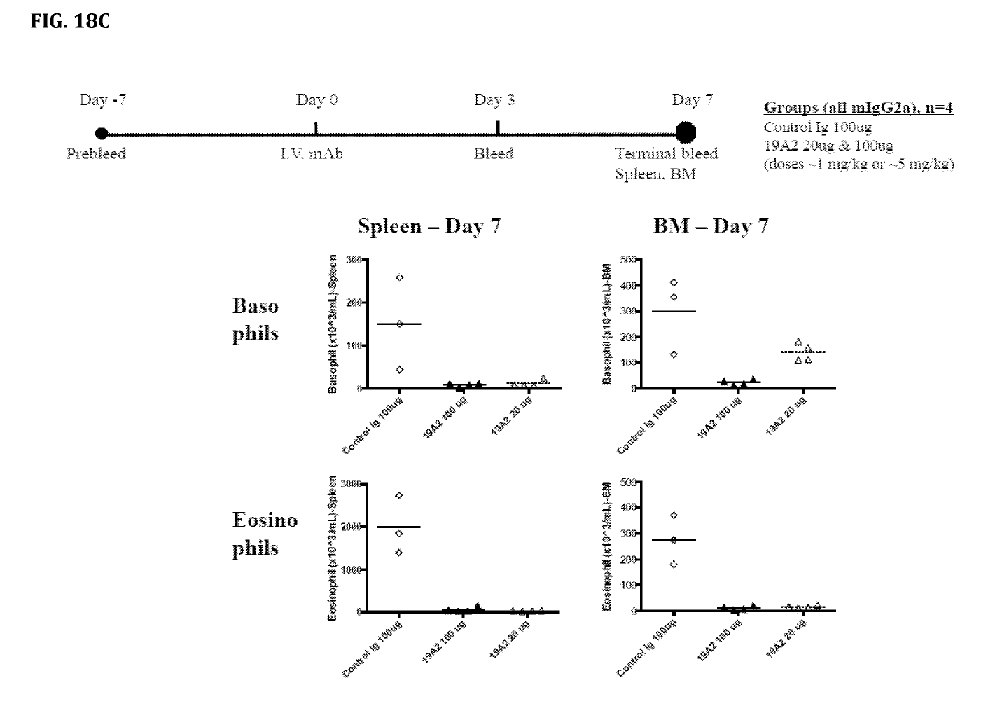

To test whether anti-CRTh2 antibody 19A2 can deplete CRTh2+ basophils and eosinophils in vivo in blood, spleen and bone marrow, a single dose of 19A2 antibody was administered to CRTh2.Bac.Tg mice as indicated (FIGS. 18A and B). A single dose of 20 ug/mouse or 100 ug/mouse of 19A2 completely depleted basophils and eosinophils in peripheral blood and spleen in human CRTh2.Bac.Tg mice on day 3, and of eosinophils in blood, spleen and bone marrow on day 7 after treatment as determined by flow cytometry (FIGS. 18A, B and C). Significant depletion of basophils was also observed at both dose levels in spleen on day 7 after treatment while the basophil depletion in bone marrow was more pronounced with the 100 ug/mouse dose. Depletion of basophils in blood was variable on day 7 after treatment.

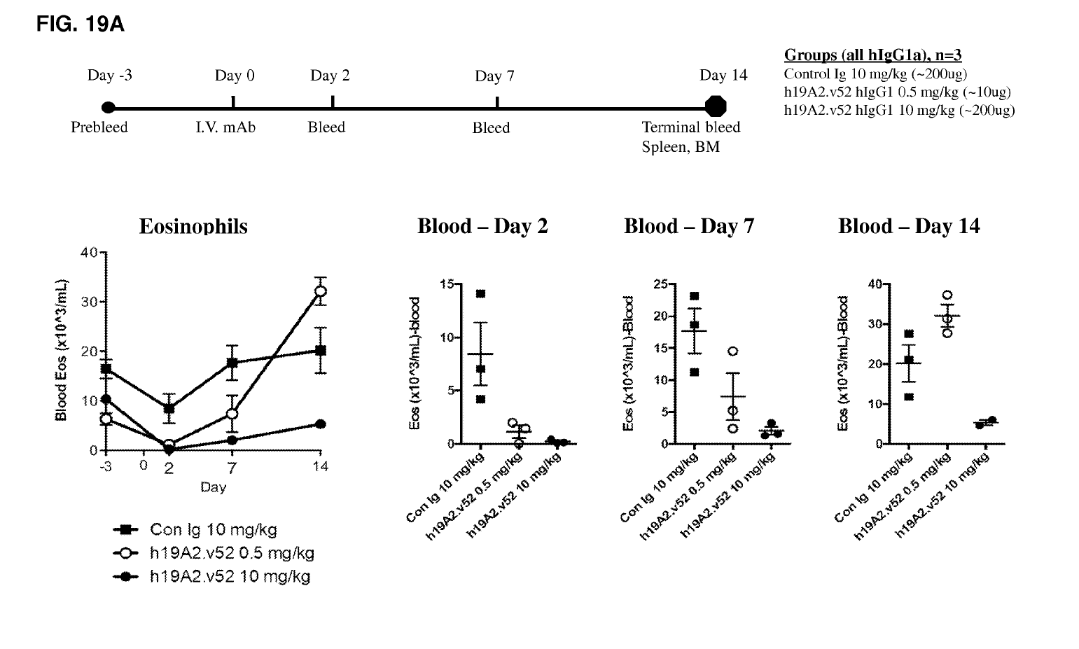

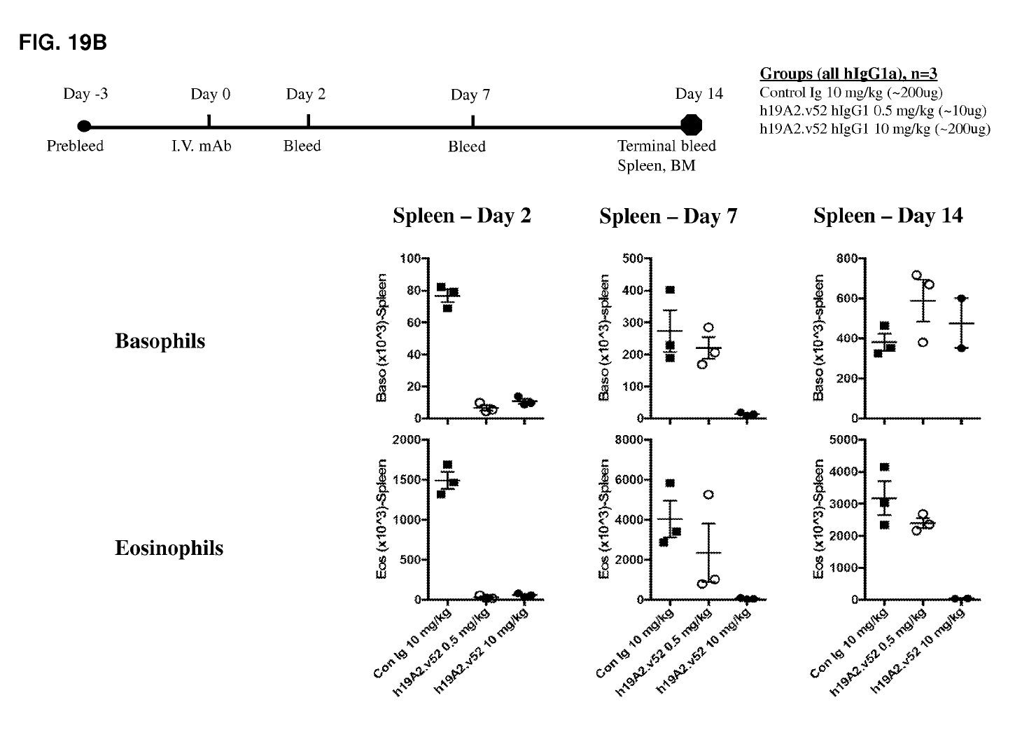

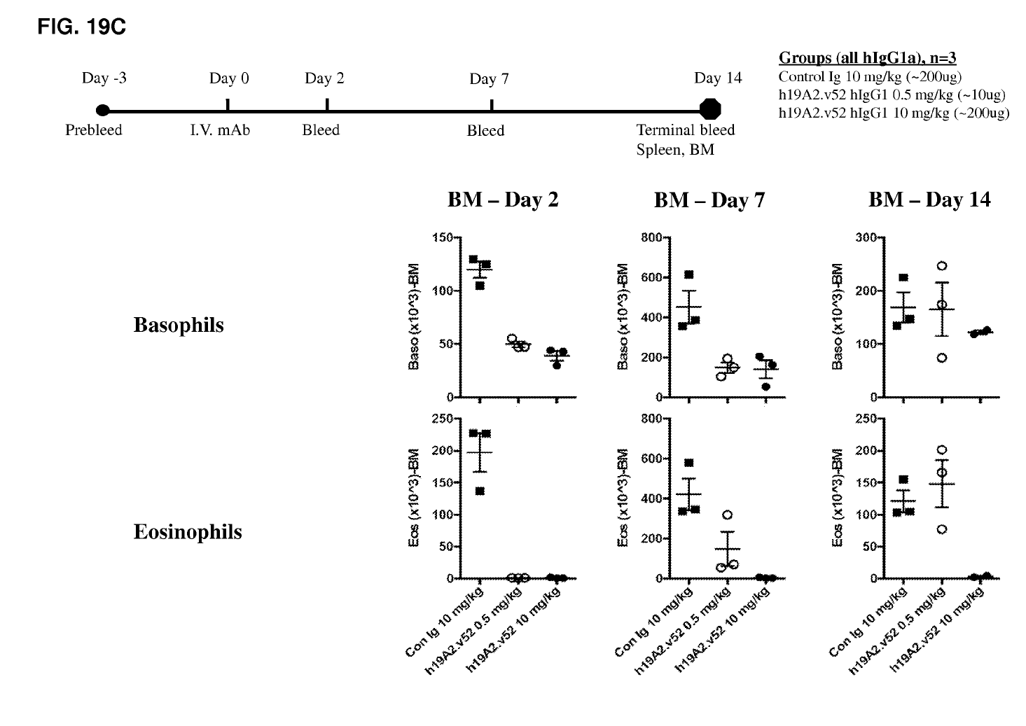

To test whether humanized and affinity matured anti-CRTh2 antibodies h19A2.v52 can deplete CRTh2+ basophils and eosinophils in vivo in blood, spleen and bone marrow, a single dose of 0.5 mg/kg or 10 mg/kg 19A2.v52 hIgG1 antibody was administered to human CRTh2.Bac.Tg mice (FIG. 19A). A single dose of 0.5 mg/kg or 10 mg/kg h19A2.v52 completely depleted eosinophils in peripheral blood, spleen or bone marrow in human CRTh2.Bac.Tg mice on day 2 as determined by flow cytometry. On day 7 and day 14 eosinophils remained depleted at the 10 mg/kg dose in spleen (FIG. 19B) and bone marrow (FIG. 19C) as well as to a large extent in blood (FIG. 19A). In comparison at the 0.5 mg/kg dose, eosinophils on day 7 partially and day 14 completely returned to baseline in blood, spleen and bone marrow. In addition, significant depletion of basophils was observed in spleen on day 2 at both dose levels and on day 7 at the 10 mg/kg dose while the basophil depletion in bone marrow was less pronounced with both doses on day 2 and day 7. Basophil levels returned to baseline at the 0.5 mg/kg dose on day 7 in spleen and at both doses on day 14 in spleen and bone marrow.

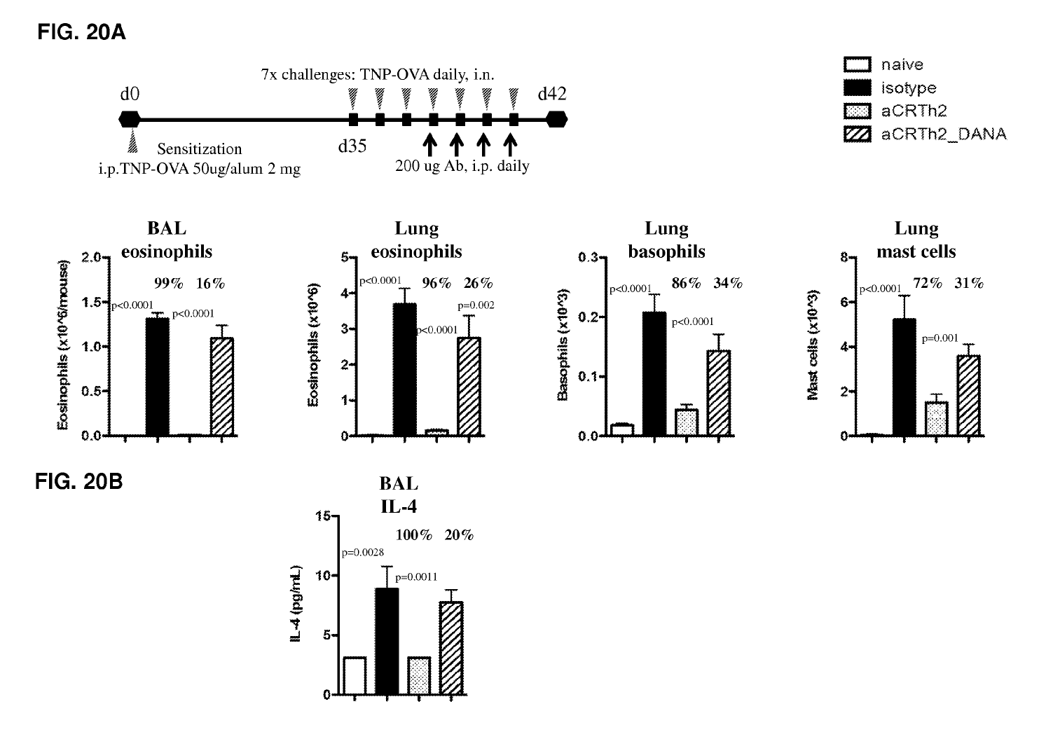

To assess whether the effector function of anti-CRTh2 antibodies are required for efficient depletion of CRTh2+ cells within tissues, the effect of the Fc mutant anti-CRTh2 19A2_DANA antibody was compared to anti-CRTh2 19A2 on innate immune cell depletion and reduction of Th2 BAL cytokine production in a TNP-OVA induced chronic asthma model in hCRTh2.Bac.Tg mice. Four doses of antibody 19A2 in a therapeutic regimen of i.p. 200 ug/mouse, depleted lung eosinophils, basophils and mast cells by 96%, 86% and 72%, respectively (FIG. 20A); in comparison 19A2_DANA treatment only partially reduced eosinophil, basophil and mast cells in lung by 26%, 34% and 31% respectively (FIG. 20A). In addition, eosinophils in the bronchial alveolar lavage (BAL) fluid were 99% depleted with 19A2 treatment but only 16% with 19A2_DANA treatment. Furthermore, the Th2 cytokines IL-4 in the BAL fluid was reduced by 100% after 19A treatment while 19A2_DANA treatment only led to a 20% reduction in BAL IL-4 (FIG. 20B).

TABLE2
Kabat CDR sequences of humanized 19A2 variants

| Antibody | | | | | | |
|---|---|---|---|---|---|---|
| Name | CDR L1 | CDR L2 | CDR L3 | CDR H1 | CDR H2 | CDR H3 |
| Hu19A2 | RASENIYXNLA | AATQLAX | QHFWITPWT | X₁YX₂MS | X₁ISNGGSTTX₂YPGTVEG | HRTNWDFDY |
| variants | X is S, W, or Y | X is D, E, or S | (SEQ ID NO: 3) | X₁ is S or F, | X₁ is Y or R, and | (SEQ ID NO: 6) |
| | (SEQ ID NO: 1) | (SEQ ID NO: 2) | | and X₂ is S, L, | X₂ is Y or D | |
| | | | | or K | (SEQ ID NO: 5) | |
| | | | | (SEQ ID NO: 4) | | |
| hu19A2.v1 | RASENIYSNLA | AATQLAD | QHFWITPWT | SYSMS | YISNGGSTTYYPGTVEG | HRTNWDFDY |
| | (SEQ ID NO: 7) | (SEQ ID NO: 10) | (SEQ ID NO: 3) | (SEQ ID NO: 13) | (SEQ ID NO: 18) | (SEQ ID NO: 6) |
| hu19A2.v12 | RASENIYWNLA | AATQLAD | QHFWITPWT | SYSMS | YISNGGSTTDYPGTVEG | HRTNWDFDY |
| | (SEQ ID NO: 8) | (SEQ ID NO: 10) | (SEQ ID NO: 3) | (SEQ ID NO: 13) | (SEQ ID NO: 19) | (SEQ ID NO: 6) |
| hu19A2.v38 | RASENIYWNLA | AATQLAD | QHFWITPWT | FYSMS | RISNGGSTTDYPGTVEG | HRTNWDFDY |
| | (SEQIDN0:8) | (SEQ ID NO: 10) | (SEQ ID NO: 3) | (SEQ ID NO: 14) | (SEQ ID NO: 20) | (SEQ ID NO: 6) |
| hu19A2.v46 | RASENIYWNLA | AATQLAD | QHFWITPWT | SYLMS | RISNGGSTTDYPGTVEG | HRTNWDFDY |
| | (SEQIDN0:8) | (SEQ ID NO: 10) | (SEQ ID NO: 3) | (SEQ ID NO: 15) | (SEQ ID NO: 20) | (SEQ ID NO: 6) |
| hu19A2.v47 | RASENIYWNLA | AATQLAD | QHFWITPWT | SYKMS | VISNGGSTTDYPGTVEG | HRTNWDFDY |
| | (SEQIDN0:8) | (SEQ ID NO: 10) | (SEQ ID NO: 3) | (SEQ ID NO: 16) | (SEQ ID NO: 21) | (SEQ ID NO: 6) |
| hu19A2.v51 | RASENIYWNLA | AATQLAD | QHFWITPWT | FYLMS | RISNGGSTTDYPGTVEG | HRTNWDFDY |
| | (SEQIDN0:8) | (SEQ ID NO: 10) | (SEQ ID NO: 3) | (SEQ ID NO: 17) | (SEQ ID NO: 20) | (SEQ ID NO: 6) |
| hu19A2.v52 | RASENIYYNLA | AATQLAD | QHFWITPWT | SYLMS | RISNGGSTTDYPGTVEG | HRTNWDFDY |
| | (SEQ ID NO: 9) | (SEQ ID NO: 10) | (SEQ ID NO: 3) | (SEQ ID NO: 15) | (SEQ ID NO: 20) | (SEQ ID NO: 6) |
| hu19A2.v53 | RASENIYYNLA | AATQLAD | QHFWITPWT | FYLMS | RISNGGSTTDYPGTVEG | HRTNWDFDY |
| | (SEQ ID NO: 9) | (SEQ ID NO: 10) | (SEQ ID NO: 3) | (SEQ ID NO: 17) | (SEQ ID NO: 20) | (SEQ ID NO: 6) |
| hu19A2.v57 | RASENIYYNLA | AATQLAE | QHFWITPWT | FYLMS | RISNGGSTTDYPGTVEG | HRTNWDFDY |
| | (SEQ ID NO: 9) | (SEQ ID NO: 11) | (SEQ ID NO: 3) | (SEQ ID NO: 17) | (SEQ ID NO: 20) | (SEQ ID NO: 6) |
| hu19A2.v58 | RASENIYYNLA | AATQLAD | QHFWITPWT | SYLMS | RISNGGSTTDYPGTVEG | HRTNWDFDY |
| | (SEQ ID NO: 9) | (SEQ ID NO: 10) | (SEQ ID NO: 3) | (SEQ ID NO: 15) | (SEQ ID NO: 20) | (SEQ ID NO: 6) |
| hu19A2.v60 | RASENIYYNLA | AATQLAE | QHFWITPWT | SYLMS | RISNGGSTTDYPGTVEG | HRTNWDFDY |
| | (SEQ ID NO: 9) | (SEQ ID NO: 11) | (SEQ ID NO: 3) | (SEQ ID NO: 15) | (SEQ ID NO: 20) | (SEQ ID NO: 6) |
| hu19A2.v61 | RASENIYYNLA | AATQLAD | QHFWITPWT | SYSMS | YISNGGSTTDYPGTVEG | HRTNWDFDY |
| | (SEQ ID NO: 9) | (SEQ ID NO: 10) | (SEQ ID NO: 3) | (SEQ ID NO: 13) | (SEQ ID NO: 19) | (SEQ ID NO: 6) |
| hu19A2.v62 | RASENIYYNLA | AATQLAE | QHFWITPWT | SYSMS | YISNGGSTTDYPGTVEG | HRTNWDFDY |
| | (SEQ ID NO: 9) | (SEQ ID NO: 11) | (SEQ ID NO: 3) | (SEQ ID NO: 13) | (SEQ ID NO: 19) | (SEQ ID NO: 6) |
| hu19A2.v63 | RASENIYWNLA | AATQLAD | QHFWITPWT | SYSMS | YISNGGSTTDYPGTVEG | HRTNWDFDY |
| | (SEQ ID NO: 8) | (SEQ ID NO: 10) | (SEQ ID NO: 3) | (SEQ ID NO: 13) | (SEQ ID NO: 19) | (SEQ ID NO: 6) |
| hu19A2.v64 | RASENIYYNLA | AATQLAD | QHFWITPWT | SYLMS | YISNGGSTTDYPGTVEG | HRTNWDFDY |
| | (SEQ ID NO: 9) | (SEQ ID NO: 10) | (SEQ ID NO: 3) | (SEQ ID NO: 15) | (SEQ ID NO: 19) | (SEQ ID NO: 6) |
| hu19A2.v65 | RASENIYWNLA | AATQLAD | QHFWITPWT | SYLMS | YISNGGSTTDYPGTVEG | HRTNWDFDY |
| | (SEQIDN0:8) | (SEQ ID NO: 10) | (SEQ ID NO: 3) | (SEQ ID NO: 15) | (SEQ ID NO: 19) | (SEQ ID NO: 6) |
| hu19A2.v66 | RASENIYYNLA | AATQLAS | QHFWITPWT | SYSMS | YISNGGSTTDYPGTVEG | HRTNWDFDY |
| | (SEQ ID NO: 9) | (SEQ ID NO: 12) | (SEQ ID NO: 3) | (SEQ ID NO: 13) | (SEQ ID NO: 19) | (SEQ ID NO: 6) |
| hu19A2.v67 | RASENIYWNLA | AATQLAS | QHFWITPWT | SYSMS | YISNGGSTTDYPGTVEG | HRTNWDFDY |
| | (SEQIDN0:8) | (SEQ ID NO: 12) | (SEQ ID NO: 3) | (SEQ ID NO: 13) | (SEQ ID NO: 19) | (SEQ ID NO: 6) |
| hu19A2.v68 | RASENIYYNLA | AATQLAS | QHFWITPWT | SYLMS | YISNGGSTTDYPGTVEG | HRTNWDFDY |
| | (SEQ ID NO: 9) | (SEQ ID NO: 12) | (SEQ ID NO: 3) | (SEQ ID NO: 15) | (SEQ ID NO: 19) | (SEQ ID NO: 6) |
| hu19A2.v69 | RASENIYWNLA | AATQLAS | QHFWITPWT | SYLMS | YISNGGSTTDYPGTVEG | HRTNWDFDY |
| | (SEQIDN0:8) | (SEQ ID NO: 12) | (SEQ ID NO: 3) | (SEQ ID NO: 15) | (SEQ ID NO: 19) | (SEQ ID NO: 6) |
| hu19A2.v70 | RASENIYSNLA | AATQLAD | QHFWITPWT | SYSMS | YISNGGSTTYYPGTVEG | HRTNWDFDY |
| | (SEQ ID NO: 7) | (SEQ ID NO: 10) | (SEQ ID NO: 3) | (SEQ ID NO: 13) | (SEQ ID NO: 18) | (SEQ ID NO: 6) |
| hu19A2.v71 | RASENIYSNLA | AATQLAE | QHFWITPWT | SYSMS | YISNGGSTTYYPGTVEG | HRTNWDFDY |
| | (SEQ ID NO: 7) | (SEQ ID NO: 11) | (SEQ ID NO: 3) | (SEQ ID NO: 13) | (SEQ ID NO: 18) | (SEQ ID NO: 6) |
| hu19A2.v72 | RASENIYSNLA | AATQLAS | QHFWITPWT | SYSMS | YISNGGSTTYYPGTVEG | HRTNWDFDY |
| | (SEQ ID NO: 7) | (SEQ ID NO: 12) | (SEQ ID NO: 3) | (SEQ ID NO: 13) | (SEQ ID NO: 18) | (SEQ ID NO: 6) |
| Mu19A2 | RASENIYSNLA | AATQLAD | QHFWITPWT | SYSMS | YISNGGSTTYYPGTVEG | HRTNWDFDY |
| | (SEQ ID NO: 7) | (SEQ ID NO: 10) | (SEQ ID NO: 3) | (SEQ ID NO: 13) | (SEQ ID NO: 18) | (SEQ ID NO: 6) |

TABLE3
Kabat CDR sequences of antibody mu8B1, hu8B1.v1, mu3C12, and mu31A5

| Antibody | | | | | | |
|---|---|---|---|---|---|---|
| Name | CDR L1 | CDR L2 | CDR L3 | CDR H1 | CDR H2 | CDR H3 |
| mu8B1 | RASQEISGYFS | AASTLDS | LQYANYPYT | ITYLIE | VIHPGSGNSHYNEKFKG | SGSSSFDYYAMDF |
| | (SEQ ID NO: | (SEQ ID | (SEQ ID NO: 27) | (SEQ ID NO: 29) | (SEQ ID NO: 32) | (SEQ ID NO: 35) |
| | 22) | NO: 25) | | | | |
| hu8B1.v1 | RASQEISGYFS | AASTLDS | LQYANYPYT | ITYLIE | VIHPGSGNSHYNEKFKG | SGSSSFDYYAMDF |
| | (SEQ ID NO: | (SEQ ID | (SEQ ID NO: 27) | (SEQ ID NO: 29) | (SEQ ID NO: 32) | (SEQ ID NO: 35) |
| | 22) | NO: 25) | | | | |
| mu3C12 | RASQEIGGYLS | AASTLDS | LQYANYPYT | TNYLID | AIHPGSGRTHYNEKFKG | SGGSSFDYYAMDY |
| | (SEQ ID NO: | (SEQ ID | (SEQ ID NO: 27) | (SEQ ID NO: 30) | (SEQ ID NO: 33) | (SEQ ID NO: 36) |
| | 23) | NO: 25) | | | | |
| mu31A5 | RASVNIYSNLA | AATNLAE | QHFWVTPYT | GYGVN | MIWDDGTTDGDSALRS | GDYGYAMDY |
| | (SEQ ID NO: | (SEQ ID | (SEQ ID NO: 28) | (SEQ ID NO: 31) | (SEQ ID NO: 34) | (SEQ ID NO: 37) |
| | 24) | NO: 26) | | | | |

MURINE ANTIBODY VARIABLE SEQUENCES
mu19A2 - Light chain variable region
(SEQ ID NO: 49)
DIQMTQSPASLSVSVGETVTITCRASENIYSNLAWYQQKQGKSPQLVVYAATQLADGVPSRFSGSGSG
TQYSLKINSLQSEDFGSYYCQHFWITPWTFGGGTKLEIK
mu19A2 - heavy chain variable region
(SEQ ID NO: 61)
EVKLVESGGGLVQPGGSLKLSCAASGFTFSSYSMSWVRQTPEKRLEWVAYISNGGSTTYYPGTVEGRF
TISRDNAKNTLFLQMSSLRSKDTAMYYCARHRTNWDFDYWGQGTTLTVSS
mu8B1 - Light chain variable region
(SEQ ID NO: 50)
DIQMTQSPSSLSASLGERVSLTCRASQEISGYFSWLQQKPDGTIKRLIYAASTLDSGVPKRFSGSRSG
SDYSLTISSLESEDFADYYCLQYANYPYTFGGGTKLEIK
mu8B1 - Heavy chain variable region
(SEQ ID NO: 62)
QVQLQQSGTGLVRPGTSVRVSCKASGYAFITYLIEWIKQRPGQGLEWIGVIHPGSGNSHYNEKFKGKA
TLTADTSSSTAYMQLSSLTSGDSAVYFCARSGSSSFDYYAMDFWGQGTSVTVSS
mu3C12 - Light chain variable region
(SEQ ID NO: 51)
DIQMTQSPSSLSASLGERVSLTCRASQEIGGYLSWLQQKPDGTFKRLIYAASTLDSGVPKRFSGSRSG
SDYSLTISSLESEDFADYYCLQYANYPYTFGGGTKLEIK
mu3C12 - Heavy chain variable region
(SEQ ID NO: 63)
QVQLQQSGADLVRPGTSVKVSCKASGYAFTNYLIDWVKQRPGQGLEWIGAIHPGSGRTHYNEKFKGKA
TLTADKSSSAAYMQISSLTSDDSAVFFCARSGGSSFDYYAMDYWGQGTSVTVSS
mu31A5 - Light chain variable sequence
(SEQ ID NO: 53)
DIQMTQSPASLSVSVGETVTITCRASVNIYSNLAWYQQRQGKSPQLLVYAATNLAEGVPSRFSGSGSG
TQYSLKINSLQSEDFGSYYCQHFWVTPYTFGGGTKLEIK
mu31A5 - Heavy chain variable sequence
(SEQ ID NO: 65)
QVQLKESGPGLVAPSQSLSITCTVSGFSLTGYGVNWVRQPPGKGLEWLGMIWDDGTTDFDSALRSRLS
ISKDNSKSQVFLKMNSLQTDDTARYFCARGDYGYAMDYWGQGTSVTVSS
HUMANIZED 8B1 VARIABLE REGION SEQUENCES
hu8B1.v1 - Light chain variable region
(SEQ ID NO: 52)
DIQMTQSPSSLSASVGDRVTITCRASQEISGYFSWLQQKPGKAPKRLIYAASTLDSGVPSRFSGSRSG
SDYTLTISSLQPEDFATYYCLQYANYPYTFGQGTKVEIK
hu8B1.v1 - Heavy chain variable region
(SEQ ID NO: 64)
EVQLVQSGAEVKKPGASVKVSCKASGYAFITYLIEWVRQAPGQGLEWIGVIHPGSGNSHYNEKFKGRA
TLTADTSTSTAYLELSSLRSEDTAVYYCARSGSSSFDYYAMDFWGQGTLVTVSS
HUMANIZED 19A2 VARIABLE REGION SEQUENCES
hu19A2.v1 - Light chain variable region
(SEQ ID NO: 38)
DIQMTQSPSSLSASVGDRVTITCRASENIYSNLAWYQQKPGKAPKLVVYAATQLADGVPSRFSGSGSG
TDYTLTISSLQPEDFATYYCQHFWITPWTFGQGTKVEIK
hu19A2.v12 - Light chain variable region
(SEQ ID NO: 39)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIK
hu19A2.v38 - Light chain variable region
(SEQ ID NO: 39)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIK
hu19A2.v46 - Light chain variable region
(SEQ ID NO: 39)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIK
hu19A2.v47 - Light chain variable region
(SEQ ID NO: 39)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIK
hu19A2.v51 - Light chain variable region
(SEQ ID NO: 39)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIK
hu19A2.v52 - Light chain variable region
(SEQ ID NO: 40)
DIQMTQSPSSLSASVGDRVTITCRASENIYYNLAWYQQKPGKAPKLVVYAATQLADGVPSRFSGSGSG
TDYTLTISSLQPEDFATYYCQHFWITPWTFGQGTKVEIK
hu19A2.v53 - Light chain variable region
(SEQ ID NO: 40)
DIQMTQSPSSLSASVGDRVTITCRASENIYYNLAWYQQKPGKAPKLVVYAATQLADGVPSRFSGSGSG
TDYTLTISSLQPEDFATYYCQHFWITPWTFGQGTKVEIK
hu19A2.v57 - Light chain variable region
(SEQ ID NO: 41)
DIQMTQSPSSLSASVGDRVTITCRASENIYYNLAWYQQKPGKAPKLLIYAATQLAEGVPSRFSGSGSG
TDYTLTISSLQPEDFATYYCQHFWITPWTFGQGTKVEIK
hu19A2.v58 - Light chain variable region
(SEQ ID NO: 42)
DIQMTQSPSSLSASVGDRVTITCRASENIYYNLAWYQQKPGKAPKLLIYAATQLADGVPSRFSGSGSG
TDYTLTISSLQPEDFATYYCQHFWITPWTFGQGTKVEIK
hu19A2.v60 - Light chain variable region
(SEQ ID NO: 41)
DIQMTQSPSSLSASVGDRVTITCRASENIYYNLAWYQQKPGKAPKLLIYAATQLAEGVPSRFSGSGSG
TDYTLTISSLQPEDFATYYCQHFWITPWTFGQGTKVEIK
hu19A2.v61 - Light chain variable region
(SEQ ID NO: 42)
DIQMTQSPSSLSASVGDRVTITCRASENIYYNLAWYQQKPGKAPKLLIYAATQLADGVPSRFSGSGSG
TDYTLTISSLQPEDFATYYCQHFWITPWTFGQGTKVEIK
hu19A2.v62 - Light chain variable region
(SEQ ID NO: 41)
DIQMTQSPSSLSASVGDRVTITCRASENIYYNLAWYQQKPGKAPKLLIYAATQLAEGVPSRFSGSGSG
TDYTLTISSLQPEDFATYYCQHFWITPWTFGQGTKVEIK
hu19A2.v63 - Light chain variable region
(SEQ ID NO: 43)
DIQMTQSPSSLSASVGDRVTITCRASENIYWNLAWYQQKPGKAPKLLIYAATQLADGVPSRFSGSGSG
TDYTLTISSLQPEDFATYYCQHFWITPWTFGQGTKVEIK
hu19A2.v64 - Light chain variable region
(SEQ ID NO: 42)
DIQMTQSPSSLSASVGDRVTITCRASENIYYNLAWYQQKPGKAPKLLIYAATQLADGVPSRFSGSGSG
TDYTLTISSLQPEDFATYYCQHFWITPWTFGQGTKVEIK
hu19A2.v65 - Light chain variable region
(SEQ ID NO: 43)
DIQMTQSPSSLSASVGDRVTITCRASENIYWNLAWYQQKPGKAPKLLIYAATQLADGVPSRFSGSGSG
TDYTLTISSLQPEDFATYYCQHFWITPWTFGQGTKVEIK
hu19A2.v66 - Light chain variable region
(SEQ ID NO: 44)
DIQMTQSPSSLSASVGDRVTITCRASENIYYNLAWYQQKPGKAPKLLIYAATQLASGVPSRFSGSGSG
TDYTLTISSLQPEDFATYYCQHFWITPWTFGQGTKVEIK
hu19A2.v67 - Light chain variable region
(SEQ ID NO: 45)
DIQMTQSPSSLSASVGDRVTITCRASENIYWNLAWYQQKPGKAPKLLIYAATQLASGVPSRFSGSGSG
TDYTLTISSLQPEDFATYYCQHFWITPWTFGQGTKVEIK
hu19A2.v68 - Light chain variable region
(SEQ ID NO: 44)
DIQMTQSPSSLSASVGDRVTITCRASENIYYNLAWYQQKPGKAPKLLIYAATQLASGVPSRFSGSGSG
TDYTLTISSLQPEDFATYYCQHFWITPWTFGQGTKVEIK
hu19A2.v69 - Light chain variable region
(SEQ ID NO: 45)
DIQMTQSPSSLSASVGDRVTITCRASENIYWNLAWYQQKPGKAPKLLIYAATQLASGVPSRFSGSGSG
TDYTLTISSLQPEDFATYYCQHFWITPWTFGQGTKVEIK
hu19A2.v70 - Light chain variable region
(SEQ ID NO: 46)
DIQMTQSPSSLSASVGDRVTITCRASENIYSNLAWYQQKPGKAPKLLIYAATQLADGVPSRFSGSGSG
TDYTLTISSLQPEDFATYYCQHFWITPWTFGQGTKVEIK
hu19A2.v71 - Light chain variable region
(SEQ ID NO: 47)
DIQMTQSPSSLSASVGDRVTITCRASENIYSNLAWYQQKPGKAPKLLIYAATQLAEGVPSRFSGSGSG
TDYTLTISSLQPEDFATYYCQHFWITPWTFGQGTKVEIK
hu19A2.v72 - Light chain variable region
(SEQ ID NO: 48)
DIQMTQSPSSLSASVGDRVTITCRASENIYSNLAWYQQKPGKAPKLLIYAATQLASGVPSRFSGSGSG
TDYTLTISSLQPEDFATYYCQHFWITPWTFGQGTKVEIK
hu19A2.v1 - Heavy Chain variable region
(SEQ ID NO: 54)
EVQLVESGGGLVQPGGSLRLSCAASGFTFSSYSMSWVRQAPGKGLEWVAYISNGGSTTYYPGTVEGRF
TISRDNSKNTLYLQMNSLRAEDTAVYYCARHRTNWDFDYWGQGTLVTVSS
hu19A2.v12 - Heavy Chain variable region
(SEQ ID NO: 55)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v38 - Heavy Chain variable region
(SEQ ID NO: 56)
EVQLVESGGGLVQPGGSLRLSCAASGFTFSFYSMSWVRQAPGKGLEWVARISNGGSTTDYPGTVEGRF
TISRDNSKNTLYLQMNSLRAEDTAVYYCARHRTNWDFDYWGQGTLVTVSS
hu19A2.v46 - Heavy Chain variable region
(SEQ ID NO: 57)
EVQLVESGGGLVQPGGSLRLSCAASGFTFSSYLMSWVRQAPGKGLEWVARISNGGSTTDYPGTVEGRF
TISRDNSKNTLYLQMNSLRAEDTAVYYCARHRTNWDFDYWGQGTLVTVSS
hu19A2.v47 - Heavy Chain variable region
(SEQ ID NO: 58)
EVQLVESGGGLVQPGGSLRLSCAASGFTFSSYKMSWVRQAPGKGLEWVAVISNGGSTTDYPGTVEGRF
TISRDNSKNTLYLQMNSLRAEDTAVYYCARHRTNWDFDYWGQGTLVTVSS
hu19A2.v51 - Heavy Chain variable region
(SEQ ID NO: 59)
EVQLVESGGGLVQPGGSLRLSCAASGFTFSFYLMSWVRQAPGKGLEWVARISNGGSTTDYPGTVEGRF
TISRDNSKNTLYLQMNSLRAEDTAVYYCARHRTNWDFDYWGQGTLVTVSS
hu19A2.v52 - Heavy Chain variable region
(SEQ ID NO: 57)
EVQLVESGGGLVQPGGSLRLSCAASGFTFSSYLMSWVRQAPGKGLEWVARISNGGSTTDYPGTVEGRF
TISRDNSKNTLYLQMNSLRAEDTAVYYCARHRTNWDFDYWGQGTLVTVSS
hu19A2.v53 - Heavy Chain variable region
(SEQ ID NO: 59)
EVQLVESGGGLVQPGGSLRLSCAASGFTFSFYLMSWVRQAPGKGLEWVARISNGGSTTDYPGTVEGRF
TISRDNSKNTLYLQMNSLRAEDTAVYYCARHRTNWDFDYWGQGTLVTVSS
hu19A2.v57 - Heavy Chain variable region
(SEQ ID NO: 59)
EVQLVESGGGLVQPGGSLRLSCAASGFTFSFYLMSWVRQAPGKGLEWVARISNGGSTTDYPGTVEGRF
TISRDNSKNTLYLQMNSLRAEDTAVYYCARHRTNWDFDYWGQGTLVTVSS
hu19A2.v58 - Heavy Chain variable region
(SEQ ID NO: 57)
EVQLVESGGGLVQPGGSLRLSCAASGFTFSSYLMSWVRQAPGKGLEWVARISNGGSTTDYPGTVEGRF
TISRDNSKNTLYLQMNSLRAEDTAVYYCARHRTNWDFDYWGQGTLVTVSS
hu19A2.v60 - Heavy Chain variable region
(SEQ ID NO: 57)
EVQLVESGGGLVQPGGSLRLSCAASGFTFSSYLMSWVRQAPGKGLEWVARISNGGSTTDYPGTVEGRF
TISRDNSKNTLYLQMNSLRAEDTAVYYCARHRTNWDFDYWGQGTLVTVSS
hu19A2.v61 - Heavy Chain variable region
(SEQ ID NO: 55)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v62 - Heavy Chain variable region
(SEQ ID NO: 55)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v63 - Heavy Chain variable region
(SEQ ID NO: 55)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v64 - Heavy Chain variable region
(SEQ ID NO: 60)
EVQLVESGGGLVQPGGSLRLSCAASGFTFSSYLMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v65 - Heavy Chain variable region
(SEQ ID NO: 60)
EVQLVESGGGLVQPGGSLRLSCAASGFTFSSYLMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v66 - Heavy Chain variable region
(SEQ ID NO: 55)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v67 - Heavy Chain variable region
(SEQ ID NO: 55)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v68
(SEQ ID NO: 60)
EVQLVESGGGLVQPGGSLRLSCAASGFTFSSYLMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v69
(SEQ ID NO: 60)
EVQLVESGGGLVQPGGSLRLSCAASGFTFSSYLMSWVRQAPGKGLEWVAYISNGGSTTDYPGTVEGRF
TISRDNSKNTLYLQMNSLRAEDTAVYYCARHRTNWDFDYWGQGTLVTVSS
hu19A2.v70 - Heavy Chain variable region
(SEQ ID NO: 54)
EVQLVESGGGLVQPGGSLRLSCAASGFTFSSYSMSWVRQAPGKGLEWVAYISNGGSTTYYPGTVEGRF
TISRDNSKNTLYLQMNSLRAEDTAVYYCARHRTNWDFDYWGQGTLVTVSS
hu19A2.v71 - Heavy Chain variable region
(SEQ ID NO: 54)
EVQLVESGGGLVQPGGSLRLSCAASGFTFSSYSMSWVRQAPGKGLEWVAYISNGGSTTYYPGTVEGRF
TISRDNSKNTLYLQMNSLRAEDTAVYYCARHRTNWDFDYWGQGTLVTVSS
hu19A2.v72 - Heavy Chain variable region
(SEQ ID NO: 54)
EVQLVESGGGLVQPGGSLRLSCAASGFTFSSYSMSWVRQAPGKGLEWVAYISNGGSTTYYPGTVEGRF
TISRDNSKNTLYLQMNSLRAEDTAVYYCARHRTNWDFDYWGQGTLVTVSS
HUMANIZED 19A2 FULL LENGTH SEQUENCES
hu19A2.v1 - Light chain
(SEQ ID NO: 66)
DIQMTQSPSSLSASVGDRVTITCRASENIYSNLAWYQQKPGKAPKLVVYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v12 - Light chain
(SEQ ID NO: 67)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v38 - Light chain
(SEQ ID NO: 67)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v46 - Light chain
(SEQ ID NO: 67)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v47 - Light chain
(SEQ ID NO: 67)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v51 - Light chain
(SEQ ID NO: 67)
DIQMTQSPSSLSASVGDRVTITCRASENIYWNLAWYQQKPGKAPKLVVYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v52 - Light chain
(SEQ ID NO: 68)
DIQMTQSPSSLSASVGDRVTITCRASENIYYNLAWYQQKPGKAPKLVVYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v53 - Light chain
(SEQ ID NO: 68)
DIQMTQSPSSLSASVGDRVTITCRASENIYYNLAWYQQKPGKAPKLVVYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v57 - Light chain
(SEQ ID NO: 69)
DIQMTQSPSSLSASVGDRVTITCRASENIYYNLAWYQQKPGKAPKLLIYAATQLAEGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v58 - Light chain
(SEQ ID NO: 70)
DIQMTQSPSSLSASVGDRVTITCRASENIYYNLAWYQQKPGKAPKLLIYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v60 - Light chain
(SEQ ID NO: 69)
DIQMTQSPSSLSASVGDRVTITCRASENIYYNLAWYQQKPGKAPKLLIYAATQLAEGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v61 - Light chain
(SEQ ID NO: 70)
DIQMTQSPSSLSASVGDRVTITCRASENIYYNLAWYQQKPGKAPKLLIYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v62 - Light chain
(SEQ ID NO: 69)
DIQMTQSPSSLSASVGDRVTITCRASENIYYNLAWYQQKPGKAPKLLIYAATQLAEGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v63 - Light chain
(SEQ ID NO: 71)
DIQMTQSPSSLSASVGDRVTITCRASENIYWNLAWYQQKPGKAPKLLIYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v64 - Light chain
(SEQ ID NO: 70)
DIQMTQSPSSLSASVGDRVTITCRASENIYYNLAWYQQKPGKAPKLLIYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v65 - Light chain
(SEQ ID NO: 71)
DIQMTQSPSSLSASVGDRVTITCRASENIYWNLAWYQQKPGKAPKLLIYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v66 - Light chain
(SEQ ID NO: 72)
DIQMTQSPSSLSASVGDRVTITCRASENIYYNLAWYQQKPGKAPKLLIYAATQLASGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v67 - Light chain
(SEQ ID NO: 73)
DIQMTQSPSSLSASVGDRVTITCRASENIYWNLAWYQQKPGKAPKLLIYAATQLASGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v68 - Light chain
(SEQ ID NO: 72)
DIQMTQSPSSLSASVGDRVTITCRASENIYYNLAWYQQKPGKAPKLLIYAATQLASGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v69 - Light chain
(SEQ ID NO: 73)
DIQMTQSPSSLSASVGDRVTITCRASENIYWNLAWYQQKPGKAPKLLIYAATQLASGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v70 - Light chain
(SEQ ID NO: 74)
DIQMTQSPSSLSASVGDRVTITCRASENIYSNLAWYQQKPGKAPKLLIYAATQLADGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v71 - Light chain
(SEQ ID NO: 75)
DIQMTQSPSSLSASVGDRVTITCRASENTYSNLAWYQQKPGKAPKLLIYAATQLAEGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v72 - Light chain
(SEQ ID NO: 76)
DIQMTQSPSSLSASVGDRVTITCRASENTYSNLAWYQQKPGKAPKLLIYAATQLASGVPSRFSGSGSG
TDYTLTISSLQPEDFATYYCQHFWITPWTFGQGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSP
VTKSFNRGEC
hu19A2.v1 - Heavy Chain
(SEQ ID NO: 77)
EVQLVESGGGLVQPGGSLRLSCAASGFTFSSYSMSWVRQAPGKGLEWVAYISNGGSTTYYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v12 - Heavy Chain
(SEQ ID NO: 78)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v38 - Heavy Chain
(SEQ ID NO: 79)
EVQLVESGGGLVQPGGSLRLSCAASGFTFSFYSMSWVRQAPGKGLEWVARISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v46 - Heavy Chain
(SEQ ID NO: 80)
EVQLVESGGGLVQPGGSLRLSCAASGFTFSSYLMSWVRQAPGKGLEWVARISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v47 - Heavy Chain
(SEQ ID NO: 81)
EVQLVESGGGLVQPGGSLRLSCAASGFTFSSYKMSWVRQAPGKGLEWVAVISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v51 - Heavy Chain
(SEQ ID NO: 82)
EVQLVESGGGLVQPGGSLRLSCAASGFTFSFYLMSWVRQAPGKGLEWVARISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v52 - Heavy Chain
(SEQ ID NO: 80)
EVQLVESGGGLVQPGGSLRLSCAASGFTFSSYLMSWVRQAPGKGLEWVARISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v53 - Heavy Chain
(SEQ ID NO: 82)
EVQLVESGGGLVQPGGSLRLSCAASGFTFSFYLMSWVRQAPGKGLEWVARISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v57 - Heavy Chain
(SEQ ID NO: 82)
EVQLVESGGGLVQPGGSLRLSCAASGFTFSFYLMSWVRQAPGKGLEWVARISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v58 - Heavy Chain
(SEQ ID NO: 80)
EVQLVESGGGLVQPGGSLRLSCAASGFTFSSYLMSWVRQAPGKGLEWVARISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v60 - Heavy Chain
(SEQ ID NO: 80)
EVQLVESGGGLVQPGGSLRLSCAASGFTFSSYLMSWVRQAPGKGLEWVARISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v61 - Heavy Chain
(SEQ ID NO: 78)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v62 - Heavy Chain
(SEQ ID NO: 78)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v63 - Heavy Chain
(SEQ ID NO: 78)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v64 - Heavy Chain
(SEQ ID NO: 83)
EVQLVESGGGLVQPGGSLRLSCAASGFTFSSYLMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v65 - Heavy Chain
(SEQ ID NO: 83)
EVQLVESGGGLVQPGGSLRLSCAASGFTFSSYLMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v66 - Heavy Chain
(SEQ ID NO: 78)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v67 - Heavy Chain
(SEQ ID NO: 78)
EVQLVESGGGLVQPGGSLRLSCAASGFTFSSYSMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v68 - Heavy Chain
(SEQ ID NO: 83)
EVQLVESGGGLVQPGGSLRLSCAASGFTFSSYLMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v69 - Heavy Chain
(SEQ ID NO: 83)
EVQLVESGGGLVQPGGSLRLSCAASGFTFSSYLMSWVRQAPGKGLEWVAYISNGGSTTDYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v70 - Heavy Chain
(SEQ ID NO: 77)
EVQLVESGGGLVQPGGSLRLSCAASGFTFSSYSMSWVRQAPGKGLEWVAYISNGGSTTYYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v71 - Heavy Chain
(SEQ ID NO: 77)
EVQLVESGGGLVQPGGSLRLSCAASGFTFSSYSMSWVRQAPGKGLEWVAYISNGGSTTYYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK
hu19A2.v72 - Heavy Chain
(SEQ ID NO: 77)
EVQLVESGGGLVQPGGSLRLSCAASGFTFSSYSMSWVRQAPGKGLEWVAYISNGGSTTYYPGTVEGRF
TISRDNSKNTLYLQMNSLRAEDTAVYYCARHRTNWDFDYWGQGTLVTVSSASTKGPSVFPLAPSSKST
SGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKA
KGQPREPQVYTLPPSREEMTKNQVSLICLVKGFYPSDIAVEWESNGQPENNYKTIPPVLDSDGSFFLY
SKLTVDKSRWQQGNVFSCSVMHEALHNHYTQKSLSLSPGK
Periostin Sequences
Human periostin isoform 1 NP_006466
(SEQ ID NO: 87)
MIPFLPMFSLLLLLIVNPINANNHYDKILAHSRIRGRDQGPNVCALQQILGTKKKYFSTCKN
WYKKSICGQKTTVLYECCPGYMRMEGMKGCPAVLPIDHVYGTLGIVGATTTQRYSDASKLRE
EIEGKGSFTYFAPSNEAWDNLDSDIRRGLESNVNVELLNALHSHMINKRMLTKDLKNGMIIP
SMYNNLGLFINHYPNGVVTVNCARIIHGNQIATNGVVHVIDRVLTQIGTSIQDFIEAEDDLS
SFRAAAITSDILEALGRDGHFTLFAPTNEAFEKLPRGVLERIMGDKVASEALMKYHILNTLQ
CSESIMGGAVFETLEGNTIEIGCDGDSITVNGIKMVNKKDIVTNNGVIHLIDQVLIPDSAKQ
VIELAGKQQTTFTDLVAQLGLASALRPDGEYTLLAPVNNAFSDDTLSMDQRLLKLILQNHIL
KVKVGLNELYNGQILETIGGKQLRVFVYRTAVCIENSCMEKGSKQGRNGAIHIFREIIKPAE
KSLHEKLKQDKRFSTFLSLLEAADLKELLTQPGDWTLFVPTNDAFKGMTSEEKEILIRDKNA
LQNIILYHLTPGVFIGKGFEPGVTNILKTTQGSKIFLKEVNDTLLVNELKSKESDIMTTNGV
IHVVDKLLYPADTPVGNDQLLEILNKLIKYIQIKFVRGSTFKEIPVTVYTTKIITKVVEPKI
KVIEGSLQPIIKTEGPTLTKVKIEGEPEFRLIKEGETITEVIHGEPIIKKYTKIIDGVPVEI
TEKETREERIITGPEIKYTRISTGGGETEETLKKLLQEEVTKVTKFIEGGDGHLFEDEEIKR
LLQGDTPVRKLQANKKVQGSRRRLREGRSQ
Human periostin isoform 2 NP_001129406
(SEQ ID NO: 88)
MIPFLPMFSLLLLLIVNPINANNHYDKILAHSRIRGRDQGPNVCALQQILGTKKKYFSTCKNWYKKSICGQKTTV
LYECCPGYMRMEGMKGCPAVLPIDHVYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYPNGVVTVNCARIIHGNQIATNGVVHV
IDRVLTQIGTSIQDFIEAEDDLSSFRAAAITSDILEALGRDGHFTLFAPTNEAFEKLPRGVLERIMGDKVASEAL
MKYHILNTLQCSESIMGGAVFETLEGNTIEIGCDGDSITVNGIKMVNKKDIVTNNGVIHLIDQVLIPDSAKQVIE
LAGKQQTTFTDLVAQLGLASALRPDGEYTLLAPVNNAFSDDTLSMDQRLLKLILQNHILKVKVGLNELYNGQILE
TIGGKQLRVFVYRTAVCIENSCMEKGSKQGRNGAIHIFREIIKPAEKSLHEKLKQDKRFSTFLSLLEAADLKELL
TQPGDWTLFVPTNDAFKGMTSEEKEILIRDKNALQNIILYHLTPGVFIGKGFEPGVTNILKTTQGSKIFLKEVND
TLLVNELKSKESDIMTTNGVIHVVDKLLYPADTPVGNDQLLEILNKLIKYIQIKFVRGSTFKEIPVTVYKPIIKK
YTKIIDGVPVEITEKETREERIITGPEIKYTRISTGGGETEETLKKLLQEEVTKVTKFIEGGDGHLFEDEEIKRL
LQGDTPVRKLQANKKVQGSRRRLREGRSQ
Human periostin isoform 3 NP_001129407
(SEQ ID NO: 89)
MIPFLPMFSLLLLLIVNPINANNHYDKILAHSRIRGRDQGPNVCALQQILGTKKKYFSTCKN
WYKKSICGQKTTVLYECCPGYMRMEGMKGCPAVLPIDHVYGTLGIVGATTTQRYSDASKLRE
EIEGKGSFTYFAPSNEAWDNLDSDIRRGLESNVNVELLNALHSHMINKRMLTKDLKNGMIIP
SMYNNLGLFINHYPNGVVTVNCARIIHGNQIATNGVVHVIDRVLTQIGTSIQDFIEAEDDLS
SFRAAAITSDILEALGRDGHFTLFAPTNEAFEKLPRGVLERIMGDKVASEALMKYHILNTLQ
CSESIMGGAVFETLEGNTIEIGCDGDSITVNGIKMVNKKDIVTNNGVIHLIDQVLIPDSAKQ
VIELAGKQQTTFTDLVAQLGLASALRPDGEYTLLAPVNNAFSDDTLSMDQRLLKLILQNHIL
KVKVGLNELYNGQILETIGGKQLRVFVYRTAVCIENSCMEKGSKQGRNGAIHIFREIIKPAE
KSLHEKLKQDKRFSTFLSLLEAADLKELLTQPGDWTLFVPTNDAFKGMTSEEKEILIRDKNA
LQNIILYHLTPGVFIGKGFEPGVTNILKTTQGSKIFLKEVNDTLLVNELKSKESDIMTTNGV
IHVVDKLLYPADTPVGNDQLLEILNKLIKYIQIKFVRGSTFKEIPVTVYRPTLTKVKIEGEP
EFRLIKEGETITEVIHGEPIIKKYTKIIDGVPVEITEKETREERIITGPEIKYTRISTGGGE
TEETLKKLLQEDTPVRKLQANKKVQGSRRRLREGRSQ
Human periostin isoform 4 NP_001129408
(SEQ ID NO: 90)
MIPFLPMFSLLLLLIVNPINANNHYDKILAHSRIRGRDQGPNVCALQQILGTKKKYFSTCKNWYKKSICGQKTTV
LYECCPGYMRMEGMKGCPAVLPIDHVYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYPNGVVTVNCARIIHGNQIATNGVVHV
IDRVLTQIGTSIQDFIEAEDDLSSFRAAAITSDILEALGRDGHFTLFAPTNEAFEKLPRGVLERIMGDKVASEAL
MKYHILNTLQCSESIMGGAVFETLEGNTIEIGCDGDSITVNGIKMVNKKDIVTNNGVIHLIDQVLIPDSAKQVIE
LAGKQQTTFTDLVAQLGLASALRPDGEYTLLAPVNNAFSDDTLSMDQRLLKLILQNHILKVKVGLNELYNGQILE
TIGGKQLRVFVYRTAVCIENSCMEKGSKQGRNGAIHIFREIIKPAEKSLHEKLKQDKRFSTFLSLLEAADLKELL
TQPGDWTLFVPTNDAFKGMTSEEKEILIRDKNALQNIILYHLTPGVFIGKGFEPGVTNILKTTQGSKIFLKEVND
TLLVNELKSKESDIMTTNGVIHVVDKLLYPADTPVGNDQLLEILNKLIKYIQIKFVRGSTFKEIPVTVYKPIIKK
YTKIIDGVPVEITEKETREERIITGPEIKYTRISTGGGETEETLKKLLQEDTPVRKLQANKKVQGSRRRLREGRS
Q
Human periostin isoform 5
(SEQ ID NO: 91)
MIPFLPMFSLLLLLIVNPINANNHYDKILAHSRIRGRDQGPNVCALQQILGTKKKYFSTCKNWYKKSICGQKTTV
LYECCPGYMRMEGMKGCPAVLPIDHVYGTLGIVGATTTQRYSDASKLREEIEGKGSFTYFAPSNEAWDNLDSDIR
RGLESNVNVELLNALHSHMINKRMLTKDLKNGMIIPSMYNNLGLFINHYPNGVVTVNCARIIHGNQIATNGVVHV
IDRVLTQIGTSIQDFIEAEDDLSSFRAAAITSDILEALGRDGHFTLFAPTNEAFEKLPRGVLERIMGDKVASEAL
MKYHILNTLQCSESIMGGAVFETLEGNTIEIGCDGDSITVNGIKMVNKKDIVTNNGVIHLIDQVLIPDSAKQVIE
LAGKQQTTFTDLVAQLGLASALRPDGEYTLLAPVNNAFSDDTLSMDQRLLKLILQNHILKVKVGLNELYNGQILE
TIGGKQLRVFVYRTAVCIENSCMEKGSKQGRNGAIHIFREIIKPAEKSLHEKLKQDKRFSTFLSLLEAADLKELL
TQPGDWTLFVPTNDAFKGMTSEEKEILIRDKNALQNITLYHLTPGVFIGKGFEPGVTNILKTTQGSKIFLKEVND
TLLVNELKSKESDIMTTNGVIHVVDKLLYPADTPVGNDQLLEILNKLIKYIQIKFVRGSTFKEIPVTVYSPEIKY
TRISTGGGETEETLKKLLQE

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An isolated antibody that binds human CRTh2 and depletes CRTh2 expressing cells when a therapeutically effective amount is administered to a human subject.

2. The antibody of claim 1, wherein the antibody depletes one or more of the following types of CRTh2 expressing cells: Th2 cells, mast cells, eosinophils, basophils, or innate type 2 (IT2) cells.

3. The antibody of claim 1, wherein the antibody has been engineered to improve ADCC and/or CDC activity.

4. The antibody of claim 1, wherein the antibody has been engineered to improve ADCC and/or reduce CDC activity.

5. The antibody of claim 1, wherein the antibody is afucosylated.

6. The antibody of claim 5, wherein the antibody is produced in a cell line having a alpha1,6-fucosyltransferase (Fut8) knockout.

7. The antibody of claim 5, wherein the antibody is produced in a cell line overexpressing β1,4-N-acetylglycosminyltransferase III (GnT-III).

8. The antibody of claim 7, wherein the cell line additionally overexpresses Golgi α-mannosidase II (ManII).

9. The antibody of claim 3, wherein the antibody comprises at least one amino acid substitution in the Fc region that improves ADCC and/or CDC activity.

10. The antibody of claim 9, wherein the amino acid substitutions are S298A/E333A/K334A.

11. The antibody of claim 1, wherein the antibody is a naked antibody.

12. The antibody of claim 1, wherein the antibody is chimeric.

13. The antibody of claim 1, wherein the antibody is humanized.

14. The antibody of claim 1, wherein the antibody is human.

15. The antibody of claim 1, wherein the antibody is a bispecific antibody.

16. The antibody of claim 1, wherein the antibody is an IgG1 antibody.

17. The antibody of claim 1, wherein the antibody competitively inhibits binding of at least one of the following antibodies: 19A2, 8B1, 31A5, and 3C12 to human CRTh2.

18. The antibody of claim 17, wherein an ELISA assay is used to determine competitive binding.

19. The antibody of claim 1, wherein the antibody binds to an epitope of human CRTh2 that is the same as or overlaps with the CRTh2 epitope bound by at least one of the following anti-CRTh2 antibodies: 19A2, 8B1, 31A5, and 3C12.

20. The antibody of claim 1, wherein the antibody comprises the six hypervariable regions (HVRs) from one of the following anti-CRTh2 antibodies: 19A2, 8B1, 31A5, and 3C12.

21. The antibody of claim 1, wherein the antibody binds to CRTh2 of a non-human primate.

22. The antibody of claim 21, wherein the antibody binds to rhesus and/or cynomologous CRTh2.

23. The antibody of claim 1, wherein the antibody further blocks CRTh2 signaling.

24. The antibody of claim 1, wherein the antibody prevents recruitment of CRTh2 expressing cells in response to prostaglandin D2.

25. The antibody of claim 1, wherein the antibody blocks Ca2⁺ flux in CRTh2 expressing cells.

26. The antibody of claim 1, wherein the antibody binds human CRTh2 with a Kd value of about 100 nM or less.

27. The antibody of claim 1, wherein the antibody comprises HVR-H3 comprising the amino acid sequence of SEQ ID NO:6, HVR-L3 comprising the amino acid sequence of SEQ ID NO:3, and HVR-H2 comprising X₁ISNGGSTTX₂YPGTVEG (SEQ ID NO:5), wherein X₁ is Y or R, and X₂ is Y or D.

28. The antibody of claim 1, wherein the antibody comprises HVR-H3 comprising the amino acid sequence of SEQ ID NO:35 or 36, HVR-L3 comprising the amino acid sequence of SEQ ID NO:27, and HVR-H2 comprising the amino acid sequence of SEQ ID NO:32 or 33.

29. An isolated anti-CRTh2 antibody comprising a light chain and heavy chain variable region, wherein the light chain and heavy chain variable region comprises six hypervariable region (HVR) sequences:
(i) HVR-L1 comprising RASENIYXNLA (SEQ ID NO:1), wherein X is S, W, or Y;
(ii) HVR-L2 comprising AATQLAX (SEQ ID NO:2), wherein X is D, E, or S;
(iii) HVR-L3 comprising QHFWITPWT (SEQ ID NO:3);
(iv) HVR-H1 comprising X₁YX₂MS (SEQ ID NO:4), wherein X₁ is S or F, and X₂ is S, L, or K;
(v) HVR-H2 comprising X₁ISNGGSTTX₂YPGTVEG (SEQ ID NO:5), wherein X₁ is Y or R, and X₂ is Y or D; and
(vi) HVR-H3 comprising HRTNWDFDY (SEQ ID NO:6).

30. An isolated anti-CRTh2 antibody comprising a light chain and heavy chain variable region, wherein the light variable region comprises HVR-L1 comprising the amino acid sequence of SEQ ID NO:7, 8, or 9, HVR-L2 comprising the amino acid sequence of SEQ ID NO:10, 11, or 12, and HVR-L3 comprising the amino acid sequence of SEQ ID NO:3.

31. The antibody of claim 29, further comprising the heavy chain variable region comprising HVR-H1 comprising the amino acid sequence of SEQ ID NO:13, 14, 15, 16, or 17, HVR-H2 comprising the amino acid sequence of SEQ ID NO:18, 19, 20, or 21, and HVR-H3 comprising amino acid sequence of SEQ ID NO:6.

32. An isolated anti-CRTh2 antibody comprising a light chain and heavy chain variable region, comprising the heavy chain variable region comprising HVR-H1 comprising the amino acid sequence of SEQ ID NO:13, 14, 15, 16, or 17, HVR-H2 comprising the amino acid sequence of SEQ ID NO:18, 19, 20, or 21, and HVR-H3 comprising amino acid sequence of SEQ ID NO:6.

33. The antibody of claim 29, wherein the antibody comprises:
(i) HVR-L1 comprising the amino acid sequence of SEQ ID NO:9;
(ii) HVR-L2 comprising the amino acid sequence of SEQ ID NO:10;
(iii) HVR-L3 comprising the amino acid sequence of SEQ ID NO:3;
(iv) HVR-H1 comprising the amino acid sequence of SEQ ID NO:15;
(v) HVR-H2 comprising the amino acid sequence of SEQ ID NO:20; and
(vi) HVR-H3 comprising the amino acid sequence of SEQ ID NO:6.

34. The antibody of claim 29, wherein the antibody comprises:
(i) HVR-L1 comprising the amino acid sequence of SEQ ID NO:8;
(ii) HVR-L2 comprising the amino acid sequence of SEQ ID NO:10;
(iii) HVR-L3 comprising the amino acid sequence of SEQ ID NO:3;
(iv) HVR-H1 comprising the amino acid sequence of SEQ ID NO:13;
(v) HVR-H2 comprising the amino acid sequence of SEQ ID NO:19; and
(vi) HVR-H3 comprising the amino acid sequence of SEQ ID NO:6.

35. The antibody of claim 29, wherein the antibody comprises:
(i) HVR-L1 comprising the amino acid sequence of SEQ ID NO:9;
(ii) HVR-L2 comprising the amino acid sequence of SEQ ID NO:11;
(iii) HVR-L3 comprising the amino acid sequence of SEQ ID NO:3;
(iv) HVR-H1 comprising the amino acid sequence of SEQ ID NO:15;
(v) HVR-H2 comprising the amino acid sequence of SEQ ID NO:20; and
(vi) HVR-H3 comprising the amino acid sequence of SEQ ID NO:6.

36. An isolated anti-CRTh2 antibody comprising a light chain and heavy chain variable region, wherein the antibody comprise a VL sequence selected from the group consisting of SEQ ID NOS:38-53.

37. The antibody of claim 36, wherein the antibody further comprises a VH sequence selected from the group consisting of SEQ ID NOS:54-65.

38. An isolated anti-CRTh2 antibody comprising a light chain and heavy chain variable region, wherein the antibody comprises a VH sequence selected from the group consisting of SEQ ID NOS:54-65.

39. The antibody of claim 36, wherein the antibody comprises a VL sequence of SEQ ID NO:40 and a VH sequence of SEQ ID NO:57.

40. The antibody of claim 36, wherein the antibody comprises a VL sequence of SEQ ID NO:39 and a VH sequence of SEQ ID NO:55.

41. The antibody of claim 36, wherein the antibody comprises a VL sequence of SEQ ID NO:41 and a VL sequence of SEQ ID NO:57.

42. The antibody of claim 29, wherein the antibody is monoclonal antibody

43. The antibody of claim 29, wherein the antibody is a humanized or chimeric antibody.

44. The antibody of claim 29, wherein at least a portion of the framework sequence is a human consensus framework sequence.

45. The antibody of claim 29, wherein the antibody is an antibody fragment selected from a Fab, Fab′-SH, Fv, scFc or (Fab′)₂ fragment.

46. An antigen-binding fragment of the antibody of claim 1.

47. An isolated nucleic acid encoding the antibody of claim 1 or an antigen-binding fragment thereof.

48. A host cell comprising the nucleic acid of claim 47.

49. A method of producing an antibody comprising culturing the host cell of claim 48 so that the antibody is produced.

50. The method of claim 49, further comprising recovering the antibody produced by the host cell.

51. An immunoconjugate comprising the antibody of claim 1 or an antigen-binding fragment thereof and a cytotoxic agent.

52. A pharmaceutical composition comprising the antibody of claim 1 or an antigen-binding fragment thereof and a pharmaceutically acceptable carrier.

53. A method for treating asthma comprising administering an effective amount of an anti-CRTh2 antibody to a subject, wherein the antibody depletes CRTh2 expressing cells in the subject.

54. The method of claim 53, wherein the antibody depletes one or more of the following types of CRTh2 expressing cells: Th2 cells, mast cells, eosinophils, basophils, or innate type 2 (IT2) cells.

55. The method of claim 53, wherein the anti-CRTh2 antibody depletes CRTh2 expressing cells from lung tissue.

56. The method of claim 53, wherein the anti-CRTh2 antibody depletes CRTh2 expressing cells from bronchioalveolar lavage fluid.

57. The method of claim 53, wherein the anti-CRTh2 antibody depletes at least 50% of at least one type of CRTh2 expressing cell from the lung compared to the baseline before administering the antibody.

58. The method of claim 57, wherein the anti-CRTh2 antibody depletes at least 80% of at least one type of CRTh2 expressing cell from the lung compared to the baseline before administering the antibody.

59. The method of claim 57, wherein the anti-CRTh2 antibody depletes at least 90% of at least one type of CRTh2 expressing cell from the lung compared to the baseline before administering the antibody.

60. The method of claim 53, wherein the subject is suffering from pauci granulocytic asthma.

61. The method of claim 53, wherein the level of one or more cytokines is reduced in the subject following administration of the anti-CRTh2 antibody.

62. The method of claim 61, wherein the level of one or more cytokines produced by at least one of the following cell types is reduced: Th2 cells, mast cells, eosinophils, basophils, or innate type 2 (IT2) cells.

63. The method of claim 61, wherein the level of one or more of IL-4, IL-5, IL-9, IL-13, IL-17, histamines or leukotrienes is reduced in the subject.

64. The method of claim 53, wherein the subject is suffering from asthma that is not adequately controlled by an inhaled corticosteroid, a short acting β2 agonist, a long acting β2 agonist, or a combination thereof.

65. The method of claim 53, wherein the subject is a human.

66. The method of claim 53, wherein the anti-CRTh2 antibody is an antibody comprising a light chain and heavy chain variable region, wherein the light chain and heavy chain variable region comprises six hypervariable region (HVR) sequences:
(i) HVR-L1 comprising RASENIYXNLA (SEQ ID NO:1), wherein X is S, W, or Y;
(ii) HVR-L2 comprising AATQLAX (SEQ ID NO:2), wherein X is D, E, or S;
(iii) HVR-L3 comprising QHFWITPWT (SEQ ID NO:3);
(iv) HVR-H1 comprising X₁YX₂MS (SEQ ID NO:4), wherein X₁ is S or F, and X₂ is S, L, or K;
(v) HVR-H2 comprising X₁ISNGGSTTX₂YPGTVEG (SEQ ID NO:5), wherein X₁ is Y or R, and X₂ is Y or D; and
(vi) HVR-H3 comprising HRTNWDFDY (SEQ ID NO:6), or an antigen-binding fragment thereof.

67. A method for treating a disorder mediated by CRTh2 expressing cells comprising administering an effective amount of an anti-CRTh2 antibody to a subject, wherein the antibody depletes CRTh2 expressing cells in the subject.

68. The method of claim 67, wherein the disorder is selected from the group consisting of: asthma, pauci granulocytic asthma, atopic dermatitis, allergic rhinitis, acute or chronic airway hypersensitivity, hypereosinophilic syndrome, eosinophilic esophagitis, Churg-Strauss syndrome, idiopathic pulmonary fibrosis, inflammation associated with a cytokine, inflammation associated with CRTh2 expressing cells, malignancy associated with CRTh2 expressing cells, chronic idiopathic urticaria, chronic spontaneous urticaria, physical urticaria, cold urticaria, pressure-urticaria, bullous pemphigoid, nasal polyposis, food allergy, and allergic bronchopulmonary aspergillosis (ABPA).

69. The method of claim 67, wherein the anti-CRTh2 antibody is an antibody comprising a light chain and heavy chain variable region, wherein the light chain and heavy chain variable region comprises six hypervariable region (HVR) sequences:
(i) HVR-L1 comprising RASENIYXNLA (SEQ ID NO:1), wherein X is S, W, or Y;
(ii) HVR-L2 comprising AATQLAX (SEQ ID NO:2), wherein X is D, E, or S;
(iii) HVR-L3 comprising QHFWITPWT (SEQ ID NO:3);
(iv) HVR-H1 comprising X₁YX₂MS (SEQ ID NO:4), wherein X₁ is S or F, and X₂ is S, L, or K;
(v) HVR-H2 comprising X₁ISNGGSTTX₂YPGTVEG (SEQ ID NO:5), wherein X₁ is Y or R, and X₂ is Y or D; and
(vi) HVR-H3 comprising HRTNWDFDY (SEQ ID NO:6), or an antigen-binding fragment thereof.

70. A method for reducing the level of a cytokine in a subject comprising administering an effective amount of an anti-CRTh2 antibody to a subject, wherein the antibody depletes CRTh2 expressing cells in the subject.

71. The method of claim 70, wherein the level of one or more IL-4, IL-5, IL-9, IL-13, IL-17, histamines or leukotrienes is reduced in the subject.

72. The method of claim 70, wherein the anti-CRTh2 antibody is an antibody comprising a light chain and heavy chain variable region, wherein the light chain and heavy chain variable region comprises six hypervariable region (HVR) sequences:
(i) HVR-L1 comprising RASENIYXNLA (SEQ ID NO:1), wherein X is S, W, or Y;
(ii) HVR-L2 comprising AATQLAX (SEQ ID NO:2), wherein X is D, E, or S;
(iii) HVR-L3 comprising QHFWITPWT (SEQ ID NO:3);
(iv) HVR-H1 comprising X₁YX₂MS (SEQ ID NO:4), wherein X₁ is S or F, and X₂ is S, L, or K;
(v) HVR-H2 comprising X₁ISNGGSTTX₂YPGTVEG (SEQ ID NO:5), wherein X₁ is Y or R, and X₂ is Y or D; and
(vi) HVR-H3 comprising HRTNWDFDY (SEQ ID NO:6), or an antigen-binding fragment thereof.
